SEQUENCE LISTING (5 sequences)

SEQ ID NO: 1 - DNA, 5686 bp, Artificial Sequence
  Human IL-12 Plasmid pIN1143
ccggccacagtcgatgaatccagaaaagcggccattttccaccatgatattcggcaagcaggcatcgccatgcgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaacaattctagaaaagccgaattctgcaggaattgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtacggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagaattgtactaacggttctttttttctcttcacaggcttaagtcatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagacgcgctagaaaagccgaattctgcaggaattgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccag

SEQ ID NO: 2 - DNA, 3426 bp, Artificial Sequence
  Mouse Interferon Alpha pIF0826
gatccatggctaggctctgtgctttcctcatgatcctagtaatgatgagctactactggtcagcctgttctctaggatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctcctagactcattctgcaatgacctccatcagcagctcaatgatctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagtgatctagaaagccgaattctgcaggaatttggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagacaacctgtagggctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgcgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagctagag

SEQ ID NO: 3 - DNA, 5966 bp, Artificial Sequence
  Mouse IL-12 pIN0961
agcttcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgcgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcggggctgcaggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccacgatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccttaactagtgaaagctcaaggccctctgccacagcgccctcctcacacagataggaattctgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccacgatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagcatgca

SEQ ID NO: 4 - DNA, 3589 bp, Artificial Sequence
  Human IFN pIF0921
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaatctagaaaagccgaattctgcaggaattgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgcgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgaattcgagcttgcatgcctgcaggt

SEQ ID NO: 5 - DNA, 567 bp, Artificial Sequence
  IFNseq DNA coding sequence for IFN-alpha2b gene in pIF0921
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

(19) United States
(12) Patent Application Publication
(10) Pub. No.: US 2003/0181405 A1
(43) Pub. Date: Sep. 25, 2003

(54) Interferon alpha plasmids and delivery systems, and methods of making and using the same

(76) Inventors: Jeffrey L. Nordstrom, College Station, TX (US); Federica Pericle, The Woodlands, TX (US); Alain Rolland, The Woodlands, TX (US); Robert Orville Ralston II, San Diego, CA (US)

Correspondence Address: PERKINS COIE LLP, POST OFFICE BOX 1208, SEATTLE, WA, 98111-1208, US

(21) Appl. No.: 10/136,837

(22) Filed: Apr. 29, 2002

Related U.S. Application Data
(63) Continuation of application No. 09/268,135, filed on Mar. 12, 1999, now abandoned.

(51) Int. Cl. A61K 48/00; A61K 38/21; C12N 15/00

(52) U.S. Cl. 514/44; 424/85.7; 435/320.1

(57) ABSTRACT

The present invention relates to gene delivery and gene therapy, and provides novel nucleic acid constructs for expression of interferon alpha in a mammal, formulations for delivery that incorporate a nucleic acid construct for expression, and methods for preparing and using such constructs and formulations. In particular, this invention relates to plasmid constructs for delivery of therapeutic interferon alpha encoding nucleic acids to cells in order to modulate tumor activity, methods of using those constructs (including combination therapy with other agents, such as cytokines, preferably IL-12), as well as methods for preparing such constructs.

### RELATED APPLICATIONS

This application is a continuation of U.S. patent application Ser. No. 09/268,135, filed Mar. 12, 1999 (Lyon & Lyon Docket No. 240/223), which relates to U.S. patent application Ser. No. 08/949,160, filed Oct. 10, 1997 and International patent application No. PCT/US97/18779, filed Oct. 10, 1997, (Lyon & Lyon Docket Nos. 226/285 US and PCT, respectively), both of which are related to U.S. patent application Serial No. 60/028,676, filed Oct. 18, 1996, (Lyon & Lyon Docket No. 222/086 US), all three of which are entitled “IL-12 GENE EXPRESSION AND DELIVERY SYSTEMS AND USES” (by Nordstrom et al.).

This application is also related to U.S. patent application Ser. No. 08/798,974, filed Feb. 11, 1997, (Lyon & Lyon Docket No. 224/084 US) and International patent application No. PCT/US95/17038, filed Dec. 28, 1995, (Lyon & Lyon Docket No. 210/190 PCT), both of which are related to U.S. patent application Ser. No. 08/372,213, filed Jan. 13, 1995, (Lyon & Lyon Docket No. 210/190 US), all three of which are entitled “FORMULATED NUCLEIC ACID COMPOSITIONS AND METHODS OF ADMINISTERING THE SAME FOR GENE THERAPY” (by Mumper Rolland).

Each of the above-mentioned applications are incorporated herein by reference in their entirety, including any drawings.

### FIELD OF THE INVENTION

The present invention relates to gene delivery and gene therapy, and provides novel nucleic acid constructs for expression of interferon alpha in a mammal, formulations for delivery that incorporate a nucleic acid construct for expression, and methods for preparing and using such constructs and formulations. In particular, this invention relates to plasmid constructs for delivery of therapeutic interferon alpha encoding nucleic acids to cells in order to modulate tumor activity, methods of using those constructs (including combination therapy with other agents, such as cytokines, preferably IL-12), as well as methods for preparing such constructs.

### BACKGROUND OF THE INVENTION

The following discussion of the background of the invention is merely provided to aid the reader in understanding the invention and is not admitted to describe or constitute prior art to the present invention.

Plasmids are an important element in genetic engineering and gene therapy. Plasmids are usually circular DNA molecules that can be introduced into bacterial cells by transformation which replicate autonomously in the cell. Plasmids typically allow for the amplification of cloned DNA. Some plasmids are present in 20 to 50 copies during cell growth, and after the arrest of protein synthesis, as many as 1000 copies per cell of a plasmid can be generated. Suzuki et al., Genetic Analysis, p. 404, 1989.

Current non-viral approaches to human gene therapy require that a potential therapeutic gene be cloned into plasmids. Large quantities of a bacterial host harboring the plasmid may be fermented and the plasmid DNA may be purified for subsequent use. Current human clinical trials using plasmids utilize this approach. Recombinant DNA Advisory Committee Data Management Report, December, 1994, Human Gene Therapy 6:535-548. Studies normally focus on the therapeutic gene and the elements that control its expression in the patient when designing and constructing gene therapy plasmids. Generally, therapeutic genes and regulatory elements are simply inserted into existing cloning vectors that are convenient and readily available.

Plasmid design and construction utilizes several key factors. First, plasmid replication origins determine plasmid copy number, which affects production yields. Plasmids that replicate to higher copy number can increase plasmid yield from a given volume of culture, but excessive copy number can be deleterious to the bacteria and lead to undesirable effects (Fitzwater, et al., Embo J. 7:3289-3297 (1988); Uhlin, et al., Mol. Gen. Genet. 165:167-179 (1979)). Artificially constructed plasmids are sometimes unstably maintained, leading to accumulation of plasmid-free cells and reduced production yields.

To overcome this problem of plasmid-free cells, genes that code for antibiotic resistance phenotype are included on the plasmid and antibiotics are added to kill or inhibit plasmid-free cells. Most general purpose cloning vectors contain ampicillin resistance (β-lactamase, or bla) genes. Use of ampicillin can be problematic because of the potential for residual antibiotic in the purified DNA, which can cause an allergic reaction in a treated patient. In addition, β-lactam antibiotics are clinically important for disease treatment. When plasmids containing antibiotic resistance genes are used, the potential exists for the transfer of antibiotic resistance genes to a potential pathogen.

Other studies have used the neo gene which is derived from the bacterial transposon Tn5. The neo gene encodes resistance to kanamycin and neomycin (Smith, Vaccine 12:1515-1519 (1994)). This gene has been used in a number of gene therapy studies, including several human clinical trials (Recombinant DNA Advisory Committee Data Management Report, December, 1994, Human Gene Therapy 6:535-548). Due to the mechanism by which resistance is imparted, residual antibiotic and transmission of the gene to potential pathogens may be less of a problem than for β-lactams.

In addition to elements that affect the behavior of the plasmid within the host bacteria, such as E. coli, plasmid vectors have also been shown to affect transfection and expression in eukaryotic cells. Certain plasmid sequences have been shown to reduce expression of eukaryotic genes in eukaryotic cells when carried in cis (Peterson, et al., Mol. Cell. Biol. 7:1563-1567 (1987); Yoder et al., Mol. Cell. Biol. 3:956-959 (1983); Lusky et al., Nature 293:79-81 (1981); and Leite, et al., Gene 82:351-356 (1989)). Plasmid sequences also have been shown to fortuitously contain binding sites for transcriptional control proteins (Ghersa, et al., Gene 151:331-332 (1994); Tully et al., Biochem. Biophys. Res. Comm. 144:1-10 (1987); and Kushner, et al., Mol. Endocrinol. 8:405-407 (1994)). This can cause inappropriate levels of gene expression in treated patients.

Interferon alpha is a gene product that has been proposed for use, either alone or in combination with other agents, in different delivery systems for the treatment of certain diseases, including particular cancers. International patent publication WO/97/00085, published Jan. 3, 1997, proposes ex vivo transfection of tumor cells with interferon alpha and another immomodulatory molecule, such as IL-12. None of the previously proposed treatments have proven entirely satisfactory, due in part to the high cost and technical difficulty involved in ex vivo approaches. Thus there still remains a need in the art for improved plasmids encoding interferon alpha as well as improved treatment protocols and technologies.

### SUMMARY

The present invention relates to gene delivery and gene therapy, and provides novel nucleic acid constructs for expression of interferon alpha in a mammal, formulations for delivery that incorporate a nucleic acid construct for expression, and methods for preparing and using such constructs and formulations. In particular, this invention relates to plasmid constructs for delivery of therapeutic interferon alpha encoding nucleic acids to cells in order to modulate tumor activity, methods of using those constructs (including combination therapy with other agents, such as cytokines, preferably IL-12), as well as methods for preparing such constructs. The pharmaceutical acceptable, cost effective and highly efficient delivery system presented herein represents an unanticipated improvement over the art.

Thus, in a first aspect, the invention features a plasmid that contains a CMV promoter and optionally a synthetic 5′ intron transcriptionally linked with an interferon alpha coding sequence, and a 3′-untranslated region (UTR). Preferably the 3′ UTR is a 3′ growth hormone UTR.

As used herein, the term “plasmid” refers to a construct made up of genetic material (i.e., nucleic acids). It includes genetic elements arranged such that an inserted coding sequence can be transcribed in eukaryotic cells. Also, while the plasmid may include a sequence from a viral nucleic acid, such viral sequence does not cause the incorporation of the plasmid into a viral particle, and the plasmid is therefore a non-viral vector. Preferably a plasmid is a closed circular DNA molecule.

“Cytomegalovirus promoter” refers to one or more sequences from a cytomegalovirus which are functional in eukaryotic cells as a transcriptional promoter and an upstream enhancer sequence. The enhancer sequence allows transcription to occur at a higher frequency from the associated promoter.

In this context, “transcriptionally linked” means that in a system suitable for transcription, transcription will initiate under the direction of the control sequence(s) and proceed through sequences which are transcriptionally linked with that control sequence(s). Preferably no mutation is created in the resulting transcript, which would alter the resulting translation product.

The term “coding region” or “coding sequence” refers to a nucleic acid sequence which encodes a particular gene product for which expression is desired, according to the normal base pairing and codon usage relationships. Thus, the coding sequence must be placed in such relationship to transcriptional control sequences (possibly including control elements and translational initiation and termination codons) that a proper length transcript will be produced and will result in translation in the appropriate reading frame to produce a functional desired product.

In a preferred embodiment the interferon alpha coding sequence is for human interferon alpha and preferably is a synthetic sequence having optimal codon usage, such as the nucleotide sequence of SEQ ID NO:11 or semi-optimal codon usage, such as the nucleotide sequence of SEQ ID NO:12.

A particular example of coding regions suitable for use in the plasmids of this invention are the natural sequences coding for human interferon alpha. Thus, in a preferred embodiment coding region has a nucleotide sequence which is the same as SEQ ID NO:10, which is the natural nucleotide sequence encoding human interferon alpha. However, it may be preferable to have an interferon alpha coding sequence which is a synthetic coding sequence. In a preferred embodiment, the interferon alpha coding sequence is a synthetic sequence utilizing optimal or semi-optimal codon usage, preferably the sequence shown in SEQ ID NO:11 or SEQ ID NO:12.

Thus, a “sequence coding for the human interferon alpha” or “a human interferon alpha coding sequence” is a nucleic acid sequence which encodes the amino acid sequence of human interferon alpha, based on the normal base pairing and translational codon usage relationships. It is preferable that the coding sequence encode the exact, full amino acid sequence of natural human interferon, but this is not essential. The encoded polypeptide may differ from natural human interferon alpha, so long as the polypeptide retains interferon alpha activity, preferably the polypeptide is at least 50%, 75%, 90%, or 97% as active as natural human interferon alpha, and more preferably fully as active as natural human interferon alpha. Thus, the polypeptide encoded by the interferon alpha coding sequence may differ from a natural human interferon alpha polypeptide by a small amount, preferably less than a 15%, 10%, 5%, or 1% change. Such a change may be of one of more different types, such as deletion, addition, or substitution of one or more amino acids.

The term “transcriptional control sequence” refers to sequences which control the rate of transcription of a transcriptionally linked coding region. Thus, the term can include elements such as promoters, operators, and enhancers. For a particular transcription unit, the transcriptional control sequences will include at least a promoter sequence.

A “growth hormone 3′ untranslated region” is a sequence located downstream (i.e., 3′) of the region encoding material polypeptide and including at least part of the sequence of the natural 3′ UTR/poly(a) signal from a growth hormone gene, preferably the human growth hormone gene. This region is generally transcribed but not translated. For expression in eukaryotic cells it is generally preferable to include sequence which signals the addition of a poly-A tail. As with other synthetic genetic elements a synthetic 3′ UTR/poly(A) signal has a sequence which differs from naturally-occurring UTR elements.

The sequence may be modified, for example by the deletion of ALU repeat sequences. Deletion of such ALU repeat sequences acts to reduce the possibility of homologous recombination between the expression cassette and genomic material in a transfected cell.

The plasmid preferably includes a promoter, a TATA box, a Cap site and a first intron and intron/exon boundary in appropriate relationship for expression of the coding sequence. The plasmid may also include a 5′ mRNA leader sequence inserted between the promoter and the coding sequence and/or an intron/5′ UTR from a chicken skeletal α-actin gene. Also, the plasmid may have a nucleotide sequence which is the same as the nucleotide sequence of plasmid pIF0921, as shown in FIGS. 5A-E.

The plasmid may also include: (a) a first transcription unit containing a first transcriptional control sequence transcriptionally linked with a first 5′-untranslated region, a first intron, a first coding sequence, and a first 3′-untranslated region/poly(A) signal, wherein the first intron is between the control sequence and the first coding sequence; and (b) a second transcription unit containing a second transcriptional control sequence transcriptionally linked with a second 5′-untranslated region, a second intron, a second coding sequence, and a second 3′-untranslated region/poly(A) signal, wherein the second intron is between the control sequence and the second coding sequence; wherein the first and second coding sequences contain a sequence having the sequence of SEQ ID NO:2, 3, 4 or 25 coding for a human IL-12 p40 subunit, and a sequence having the sequence of SEQ ID NO:6, 7, 8 or 24 coding for a human IL-12 p35 subunit.

The term “transcription unit” or “expression cassette” refers to a nucleotide sequence which contains at least one coding sequence along with sequence elements which direct the initiation and termination of transcription. A transcription unit may however include additional sequences, which may include sequences involved in post-transcriptional or post-translational processes. In preferred embodiments, the first transcriptional control sequence or the second transcriptional control sequence contain one or more cytomegalovirus promoter sequences. The first and second transcriptional control sequences can be the same or different.

A “5′ untranslated region” or “5′ UTR” refers to a sequence located 3′ to promoter region and 5′ of the downstream coding region. Thus, such a sequence, while transcribed, is upstream of the translation initiation codon and therefore is not translated into a portion of the polypeptide product.

For the plasmids described herein, one or more of a promoter, 5′ untranslated region (5′ UTR), the 3′ UTR/poly(A) signal, and introns may be a synthetic sequence. In this context the term “synthetic” means that the sequence is not provided directly by the sequence of a naturally occurring genetic element of that type but rather is an artificially created sequence (i.e., created by a person by molecular biological methods). While one or more portions of such a synthetic sequence may be the same as portions of naturally occurring sequences, the full sequence over the specified genetic element is different from a naturally occurring genetic element of that type. The use of such synthetic genetic elements allows the functional characteristics of that element to be appropriately designed for the desired function.

Thus, a “synthetic intron” refers to a sequence which is not a naturally occurring intron sequence but which will be removed from an RNA transcript during normal post transcriptional processing. Such introns can be designed to have a variety of different characteristics, in particular such introns can be designed to have a desired strength of splice site.

A “subunit” of a therapeutic molecule is a polypeptide or RNA molecule which combines with one or more other molecules to form a complex having the relevant pharmacologic activity. Examples of such complexes include homodimers and heterodimers as well as complexes having greater numbers of subunits. A specific example of a heterodimer is IL-12, having the p40 and p35 subunits.

The “p40 subunit” is the larger of the two subunits of the IL-12 heterodimer.

Thus, it is capable of association with p35 subunit to form a molecule having activities characteristic of IL-12. Human p40 has the amino acid sequence of SEQ ID NO:1. Those skilled in the art will recognize that the molecule may have a number of changes from that sequence, such as deletions, insertions or changes of one or a few amino acids, while still retaining IL-12 activity when associated with p35. Such active altered molecules are also regarded as p40.

Conversely, the “p35 subunit” is the smaller of the two heterodimeric subunits of IL-12. For humans, p35 has the amino acid sequence of SEQ ID NO:5. As for p40, p35 may have a low level of alterations from that sequence while still being regarded as p35.

A particular example of coding regions suitable for use in the plasmids of this invention are the natural sequences coding for the p40 and p35 subunits of human IL-12. Thus, in a preferred embodiment the first and second coding regions are coding regions for those sequences and are preferably in the order p40 then p35 in the 5′ to 3′ direction.

Thus, a “sequence coding for the p40 subunit of human IL-12” is a nucleic acid sequence which encodes the human p40 subunit as described above, based on the normal base pairing and translational codon usage relationships. The sequence coding for p35 subunit of human IL-12 is similarly defined.

In a preferred embodiment the sequence coding for the p40 subunit of human IL-12 is 5′ to the sequence coding for the p35 subunit of human IL-12. Those skilled in the art will appreciate that the interferon alpha, p35 subunit and p40 subunit may all be on a single transcription unit, that all three may be on separate transcription units, or that any two coding sequences may be on one transcription unit and the other coding sequence on another transcription unit.

The plasmid may also contain an intron having variable splicing, a first coding sequence, and a second coding sequence, wherein the first and second coding sequences include a sequence having the sequence of SEQ ID NO:2, 3, 4 or 25 coding for a human IL-12 p40 subunit, and a sequence having the sequence of SEQ ID NO:6, 7, 8 or 24 coding for a human IL-12 p35 subunit.

In a preferred embodiment, the plasmid also has: (a) a transcriptional control sequence transcriptionally linked with a first coding sequence and a second coding sequence; (b) a 5′-untranslated region; (c) an intron 5′ to the first coding sequence; (d) an alternative splice site 3′ to the first coding sequence and 5′ to the second coding sequence; and (e) a 3′-untranslated region/poly(A) signal. The transcriptional control sequence preferably includes a cytomegalovirus promoter sequence.

In a preferred embodiment, the plasmid also has: (a) a transcriptional control sequence transcriptionally linked with a first coding sequence, an IRES sequence, a second coding sequence, and a 3′-untranslated region/poly(A) signal, wherein the IRES sequence is between the first coding sequence and the second coding sequence; and (b) an intron between the promoter and the first coding sequence; wherein the first and second coding sequences include a sequence having the sequence of SEQ ID NO:2, 3, 4 or 25 coding for a human IL-12 p40 subunit, and a sequence having the sequence of SEQ ID NO:6, 7, 8 or 24 coding for a human IL-12 p35 subunit. The transcriptional control sequence preferably includes a cytomegalovirus promoter sequence and the IRES sequence preferably is from an encephalomyocarditis virus.

For delivery of coding sequences for gene expression, it is generally useful to provide a delivery composition or delivery system which includes one or more other components in addition to the nucleic acid sequences. Such a composition can, for example, aid in maintaining the integrity of the DNA and/or in enhancing cellular uptake of the DNA and/or by acting as an immunogenic enhancer, such as by the non-DNA components having an immuno-stimulatory effect of their own.

Thus, in another aspect, the invention features a composition containing a plasmid as described above and a protective, interactive non-condensing compound (PINC).

The PINC enhances the delivery of the nucleic acid molecule to mammalian cells in vivo, and preferably the nucleic acid molecule includes a coding sequence for a gene product to be expressed in the cell. In many cases, the relevant gene product is a polypeptide or protein. Preferably the PINC is used under conditions so that the PINC does not form a gel, or so that no gel form is present at the time of administration at about 30-40° C. Thus, in these compositions, the PINC is present at a concentration of 30% (w/v) or less. In certain preferred embodiments, the PINC concentration is still less, for example, 20% or less, 10% or less, 5% or less, or 1% or less. Thus, these compositions differ in compound concentration and functional effect from uses of these or similar compounds in which the compounds are used at higher concentrations, for example in the ethylene glycol mediated transfection of plant protoplasts, or the formation of gels for drug or nucleic acid delivery. In general, the PINCs are not in gel form in the conditions in which they are used as PINCs, though certain of the compounds may form gels under some conditions.

In connection with the compounds and compositions of this invention, the term “protects” or “protective” refers to an effect of the interaction between such a compound and a nucleic acid such that the rate of degradation of the nucleic acid is decreased in a particular environment. Such degradation may be due to a variety of different factors, which specifically include the enzymatic action of a nuclease. The protective action may be provided in different ways, for example, by exclusion of the nuclease molecules or by exclusion of water.

Some compounds which protect a nucleic acid and/or prolong the bioavailability of a nucleic acid may also interact or associate with the nucleic acid by intermolecular forces and/or valence bonds such as: Van der Waals forces, ion-dipole interactions, ion-induced dipole interactions, hydrogen bonds, or ionic bonds. These interactions may serve the following functions: (1) Stereoselectively protect nucleic acids from nucleases by shielding; (2) facilitate the cellular uptake of nucleic acid by “piggyback endocytosis”. Piggyback endocytosis is the cellular uptake of a drug or other molecule complexed to a carrier that may be taken up by endocytosis. CV Uglea and C Dumitriu-Medvichi, Medical Applications of Synthetic Oligomers, In: Polymeric Biomaterials, Severian Dumitriu ed., Marcel Dekker, Inc., 1993, incorporated herein by reference.

To achieve the desired effects set forth it is desirable, but not necessary, that the compounds which protect the nucleic acid and/or prolong the bioavailability of a nucleic acid have amphiphilic properties; that is, have both hydrophilic and hydrophobic regions. The hydrophilic region of the compounds may associate with the largely ionic and hydrophilic regions of the nucleic acid, while the hydrophobic region of the compounds may act to retard diffusion of nucleic acid and to protect nucleic acid from nucleases.

Additionally, the hydrophobic region may specifically interact with cell membranes, possibly facilitating endocytosis of the compound and thereby also of nucleic acid associated with the compound. This process may increase the pericellular concentration of nucleic acid.

Agents which may have amphiphilic properties and are generally regarded as being pharmaceutically acceptable are the following: polyvinylpyrrolidones; polyvinylalcohols; polyvinylacetates; propylene glycol; polyethylene glycols; poloxamers (Pluronics); poloxamines (Tetronics); ethylene vinyl acetates; methylcelluloses, hydroxypropylcelluloses, hydroxypropylmethylcelluloses; heteropolysaccharides (pectins); chitosans; phosphatidylcholines (lecithins); miglyols; polylactic acid; polyhydroxybutyric acid; xanthan gum. Also, copolymer systems such as polyethylene glycol-polylactic acid (PEG-PLA), polyethylene glycol-polyhydroxybutyric acid (PEG-PHB), polyvinylpyrrolidone-polyvinylalcohol (PVP-PVA), and derivatized copolymers such as copolymers of N-vinyl purine (or pyrimidine) derivatives and N-vinylpyrrolidone. However, not all of the above compounds are protective, interactive, non-condensing compounds as described below.

In connection with the protective, interactive, non-condensing compounds for these compositions, the term “non-condensing” means that an associated nucleic acid is not condensed or collapsed by the interaction with the PINC at the concentrations used in the compositions. Thus, the PINCs differ in type and/or use concentration from such condensing polymers. Examples of commonly used condensing polymers include polylysine, and cascade polymers (spherical polycations).

Also in connection with such compounds and an associated nucleic acid molecule, the term “enhances the delivery” means that at least in conditions such that the amounts of PINC and nucleic acid is optimized, a greater biological effect is obtained than with the delivery of nucleic acid in saline. Thus, in cases where the expression of a gene product encoded by the nucleic acid is desired, the level of expression obtained with the PINC:nucleic acid composition is greater than the expression obtained with the same quantity of nucleic acid in saline for delivery by a method appropriate for the particular PINC/coding sequence combination.

In preferred embodiments of the above compositions, the PINC is polyvinyl pyrrolidone (PVP), polyvinyl alcohol (PVA), a PVP-PVA co-polymer, N-methyl-2-pyrrolidone (NM2P), ethylene glycol, or propylene glycol. In compositions in which a Poloxamer (Pluronics) is used, the nucleic acid is preferably not a viral vector, i.e., the nucleic acid is a non-viral vector.

In other preferred embodiments, the PINC is bound with a targeting ligand. Such targeting ligands can be of a variety of different types, including but not limited to galactosyl, residues, fucosal residues, mannosyl residues, camtitine derivatives, monoclonal antibodies, polyclonal antibodies, peptide ligands, and DNA-binding proteins. The targeting ligands may bind with receptors on cells such as antigen-presenting cells, hepatocytes, myocytes, epithelial cells, endothelial cells, and cancer cells.

In connection with the association of a targeting ligand and a PINC, the term “bound with” means that the parts have an interaction with each other such that the physical association is thermodynamically favored, representing at least a local minimum in the free energy function for that association. Such interaction may involve covalent binding, or non-covalent interactions such as ionic, hydrogen bonding, van der Waals interactions, hydrophobic interactions, and combinations of such interactions.

While the targeting ligand may be of various types, in one embodiment the ligand is an antibody. Both monoclonal antibodies and polyclonal antibodies may be utilized.

The nucleic acid may also be present in various forms. Preferably the nucleic acid is not associated with a compounds(s) which alter the physical form, however, in other embodiments the nucleic acid is condensed (such as with a condensing polymer), formulated with cationic lipids, formulated with peptides, or formulated with cationic polymers.

In preferred embodiments, the protective, interactive non-condensing compound is polyvinyl pyrrolidone, and/or the plasmid is in a solution having between 0.5% and 50% PVP, more preferably about 5% PVP. The DNA preferably is at least about 80% supercoiled, more preferably at least about 90% supercoiled, and most preferably at least about 95% supercoiled.

In another aspect the invention features a composition containing a protective, interactive non-condensing compound and a plasmid containing an interferon alpha coding sequence.

In yet another aspect, the invention provides a composition containing a plasmid of the invention (or a plasmid containing an interferon alpha coding sequence) and a cationic lipid with a neutral co-lipid.

Preferably the cationic lipid is DOTMA and the neutral co-lipid is cholesterol (chol). DOTMA is 1,2-di-O-octadecenyl-3-trimethylammonium propane, which is described and discussed in Eppstein et al., U.S. Pat. No. 4,897,355, issued Jan. 30, 1990, which is incorporated herein by reference. However, other lipids and lipid combinations may be used in other embodiments. A variety of such lipids are described in Gao & Huang, 1995, Gene Therapy 2:710-722, which is hereby incorporated by reference.

As the charge ratio of the cationic lipid and the DNA is also a significant factor, in preferred embodiments the DNA and the cationic lipid are present is such amounts that the negative to positive charge ratio is about 1:3. While preferable, it is not necessary that the ratio be 1:3. Thus, preferably the charge ratio for the compositions is between about 1:1 and 1:10, more preferably between about 1:2 and 1:5.

The term “cationic lipid” refers to a lipid which has a net positive charge at physiological pH, and preferably carries no negative charges at such pH. An example of such a lipid is DOTMA. Similarly, “neutral co-lipid” refers to a lipid which has is usually uncharged at physiological pH. An example of such a lipid is cholesterol.

Thus, “negative to positive charge ratio” for the DNA and cationic lipid refers to the ratio between the net negative charges on the DNA compared to the net positive charges on the cationic lipid.

As the form of the DNA affects the expression efficiency, the DNA preferably is at least about 80% supercoiled, more preferably at least about 90% supercoiled, and most preferably at least about 95% supercoiled. The composition preferably includes an isotonic carbohydrate solution, such as an isotonic carbohydrate solution that consists essentially of about 10% lactose. In preferred embodiments, the composition the cationic lipid and the neutral co-lipid are prepared as a liposome having an extrusion size of about 800 nanometers. Preferably the liposomes are prepared to have an average diameter of between about 20 and 800 nm, more preferably between about 50 and 400 nm, still more preferably between about 75 and 200 nm, and most preferably about 100 nm. Microfluidization is the preferred method of preparation of the liposomes.

In another aspect the invention features a composition containing: (a) a first component having a plasmid including an interferon alpha coding sequence and a cationic lipid with a neutral co-lipid, wherein the cationic lipid is DOTMA and the neutral co-lipid is cholesterol, wherein the DNA in the plasmid and the cationic lipid are present in amounts such that the negative to positive charge ratio is about 1:3; and (b) a second component including a protective, interactive non-condensing compound, wherein the first component is present within the second component.

In another aspect, the invention provides a composition having a protective, interactive non-condensing compound, a first plasmid including an interferon alpha coding sequence, and one or more other plasmids independently having an IL-12 p35 or IL-12 p40 subunit coding sequence.

In another aspect, the invention features a method for making any of the plasmids described above by inserting a CMV promoter transcriptionally linked with an interferon alpha coding sequence, and a growth hormone 3′-untranslated region into a plasmid.

The invention also provides methods of making the compositions described above. The method may involve: (a) preparing a DNA molecule having a transcriptional unit, wherein the transcriptional unit contains an interferon alpha coding sequence; (b) preparing a protective, interactive non-condensing compound; and (c) combining the protective, interactive non-condensing compound with the DNA in conditions such that a composition capable of delivering a therapeutically effective amount of an interferon alpha coding sequence to a mammal is formed.

Preferably, the DNA molecule is a plasmid, wherein the plasmid includes a CMV promoter transcriptionally linked with an interferon alpha coding sequence, and a human growth hormone 3′-untranslated region/poly(A) signal.

The method may involve the steps of: (a) preparing a DNA having an interferon alpha coding sequence; (b) preparing a mixture of a cationic lipid and a neutral co-lipid, wherein the cationic lipid is DOTMA and the neutral co-lipid is cholesterol; and (c) combining the mixture with the DNA in amounts such that the cationic lipid and the DNA are present in a negative to positive charge ratio of about 1:3.

In another embodiment, the method involves the steps of: (a) preparing a first component having a plasmid containing an interferon alpha coding sequence and a cationic lipid with a neutral co-lipid, wherein the cationic lipid is DOTMA and the neutral co-lipid is cholesterol, wherein the DNA in the plasmid and the cationic lipid are present in amounts such that the negative to positive charge ratio is about 1:3; (b) preparing a second component having a protective, interactive non-condensing compound; and (c) combining the first and second components such that the resulting composition includes the first component within the second component.

In another embodiment, the method involves the steps of: (a) preparing a protective, interactive non-condensing compound, (b) preparing a first plasmid having an interferon alpha coding sequence, (c) preparing one or more other plasmids independently having an IL-12 p35 or IL-12 p40 subunit coding sequence, and (d) combining the protective, interactive non-condensing compound, the plasmid having the interferon alpha coding sequence and the other plasmids.

In another aspect, the invention provides a method for treatment of a mammalian condition or disease, by administering to a mammal suffering from the condition or disease a therapeutically effective amount of a plasmid as described herein.

A “therapeutically effective amount” of a composition is an amount which is sufficient to cause at least temporary relief or improvement in a symptom or indication of a disease or condition. Thus, the amount is also sufficient to cause a pharmacological effect. The amount of the composition need not cause permanent improvement or improvement of all symptoms or indications. A therapeutically effective amount of a cancer therapeutic would be one that reduces overall tumor burden in the case of metastatic disease (i.e., the number of metasteses or their size) or one that reduces the mass of the tumor in localized cancers.

The condition or disease preferably is a cancer, such as epithelial glandular cancer, including adenoma and adenocarcinoma; squamous and transitional cancer, including polyp, papilloma, squamous cell and transitional cell carcinoma; connective tissue cancer, including tissue type positive, sarcoma and other (oma's); hematopoietic and lymphoreticular cancer, including lymphoma, leukemia and Hodgkin's disease; neural tissue cancer, including neuroma, sarcoma, neurofibroma and blastoma; mixed tissues of origin cancer, including teratoma and teratocarcinoma. Other cancerous conditions that are applicable to treatment include cancer of any of the following: adrenal gland, anus, bile duct, bladder, brain tumors: adult, breast, cancer of an unknown primary site, carcinoids of the gastrointestinal tract, cervix, childhood cancers, colon and rectum, esophagus, gall bladder, head and neck, islet cell and other pancreatic carcinomas, Kaposi's sarcoma, kidney, leukemia, liver, lung: non-small cell, lung: small cell, lymphoma: AIDS-associated, lymphoma: Hodgkin's disease, Lymphomas: non-Hodgkin's disease, melanoma, mesothelioma, metastatic cancer, multiple myeloma, ovary, ovarian germ cell tumors, pancreas, parathyroid, penis, pituitary, prostate, sarcomas of bone and soft tissue, skin, small intestine, stomach, testis, thymus, thyroid, trophoblastic disease, uterus: endometrial carcinoma, uterus: uterine sarcomas, vagina, or vulva. The composition preferably is administered by injection, although the method may also be performed ex vivo.

In another aspect, the invention provides a method for transfection (i.e., the delivery and expression of a gene to cells) of a cell in situ, by contacting the cell with a plasmid described herein for sufficient time to transfect the cell. Transfection of the cell preferably is performed in vivo and the contacting preferably is performed in the presence of about 5% PVP solution.

In another aspect, the invention features a method for delivery and expression of an interferon alpha gene in a plurality of cells, by: (a) transfecting the plurality of cells with a plasmid or composition of the invention; and (b) incubating the plurality of cells under conditions allowing expression of a nucleic acid sequence in the vector, wherein the nucleic acid sequence encodes interferon alpha.

In preferred embodiments, the interferon alpha is human interferon alpha and the cells are human cells and/or the contacting is performed in the presence of an about 5% PVP solution.

In another aspect, the invention features a method for treating a disease or condition, by transfecting a cell in situ with a plasmid or composition of the invention. The disease or condition can be a localized disease or condition or a systemic disease or condition.

In another aspect, the invention features a cell transfected with a plasmid or composition of the invention.

In yet another aspect, the invention features a method for treatment of a mammalian condition or disease, by administering to a mammal suffering from the condition or disease a therapeutically effective amount of a composition described herein.

As the compositions are useful for delivery of a nucleic acid molecule to cells in vivo, in a related aspect the invention provides a composition at an in vivo site of administration. In particular this includes at an in vivo site in a mammal.

In preferred embodiments the nucleic acid molecule includes a sequence encoding a gene product. Also in preferred embodiments, the site of administration is in an interstitial space or a tissue of an animal, particularly of a mammal.

The invention also provides methods for using the above compositions. Therefore, in further related aspects, methods of administering the compositions are provided in which the composition is introduced into a mammal, preferably into a tissue or an interstitial space.

Various methods of delivery may be utilized, such as are known in the art, but in preferred embodiments, the composition is introduced into the tissue or interstitial space by injection. The compositions may also be delivered to a variety of different tissues, but in preferred embodiments the tissue is muscle or tumor.

In another related aspect, the invention provides methods for treating a mammalian condition or disease by administering a therapeutically effective amount of a composition as described above. In preferred embodiments, the disease or condition is a cancer.

The summary of the invention described above is non-limiting and other features and advantages of the invention will be apparent from the following detailed description of the preferred embodiments, as well as from the claims.

### BRIEF DESCRIPTION OF THE DRAWINGS

FIG. 1 shows the effects of interferon alpha in two cancer models.

FIGS. 2A-E show a plasmid map and sequence (SEQ ID NO:18) for an exemplary IL-12 plasmid of the present invention.

FIGS. 3A-B show optimal codon usage for highly expressed human genes.

FIGS. 4A-C show a plasmid map and sequence (SEQ ID NO:19) for plasmid pIF0836, an exemplary interferon alpha plasmid of the present invention.

FIGS. 5A-E show a plasmid map and sequence (SEQ ID NO:20) for pIN096, an exemplary IL-12 plasmid that can be used with the present invention.

FIGS. 6A-B show the nucleic acid sequence (SEQ ID NO:21) of plasmid pIF0921, an exemplary interferon alpha plasmid of the present invention.

FIGS. 7A-B show a plasmid map and sequence (SEQ ID NO:22) for plasmid pIF0921.

FIG. 8 shows an outline of a strategy that can be used to synthesize a pIF0921 plasmid.

FIG. 9 shows interferon alpha and IL-12 gene medicine (combination therapy) in Renca model.

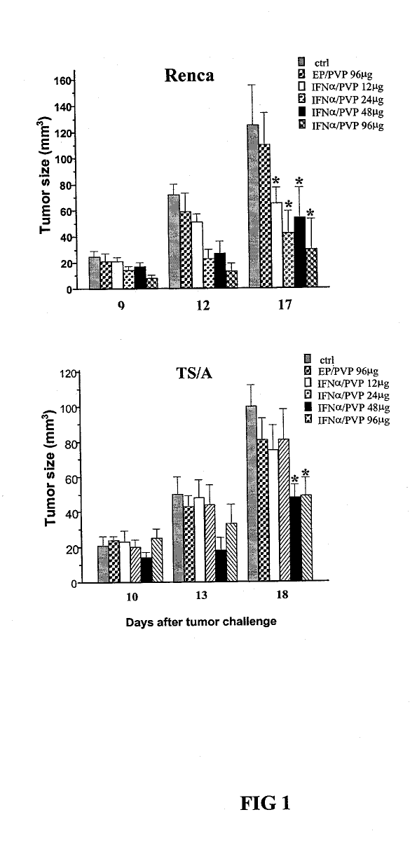

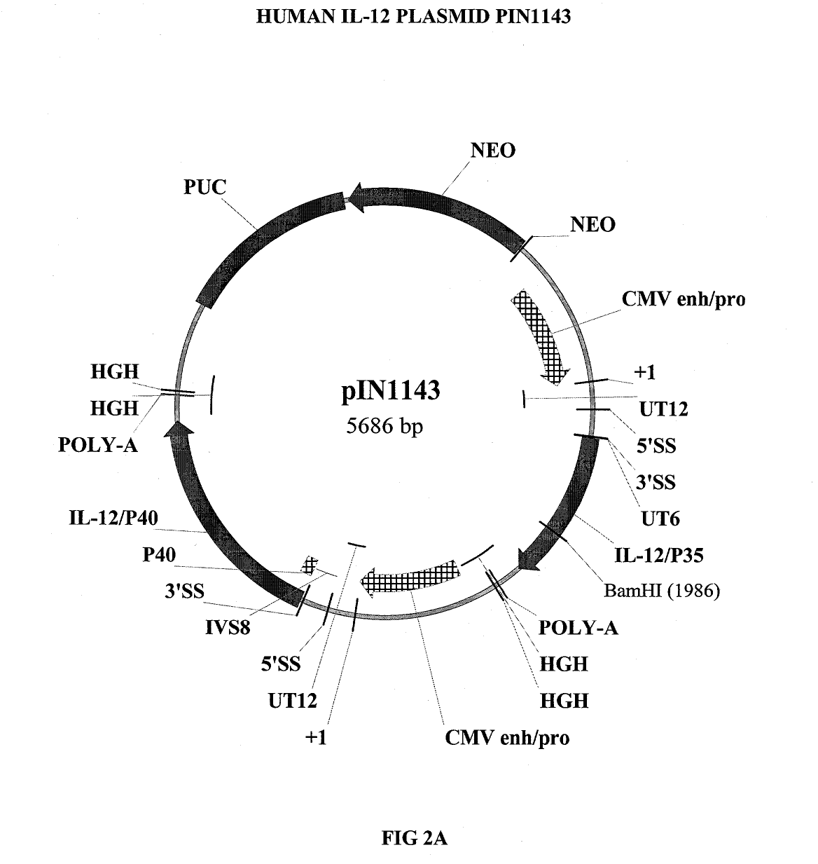

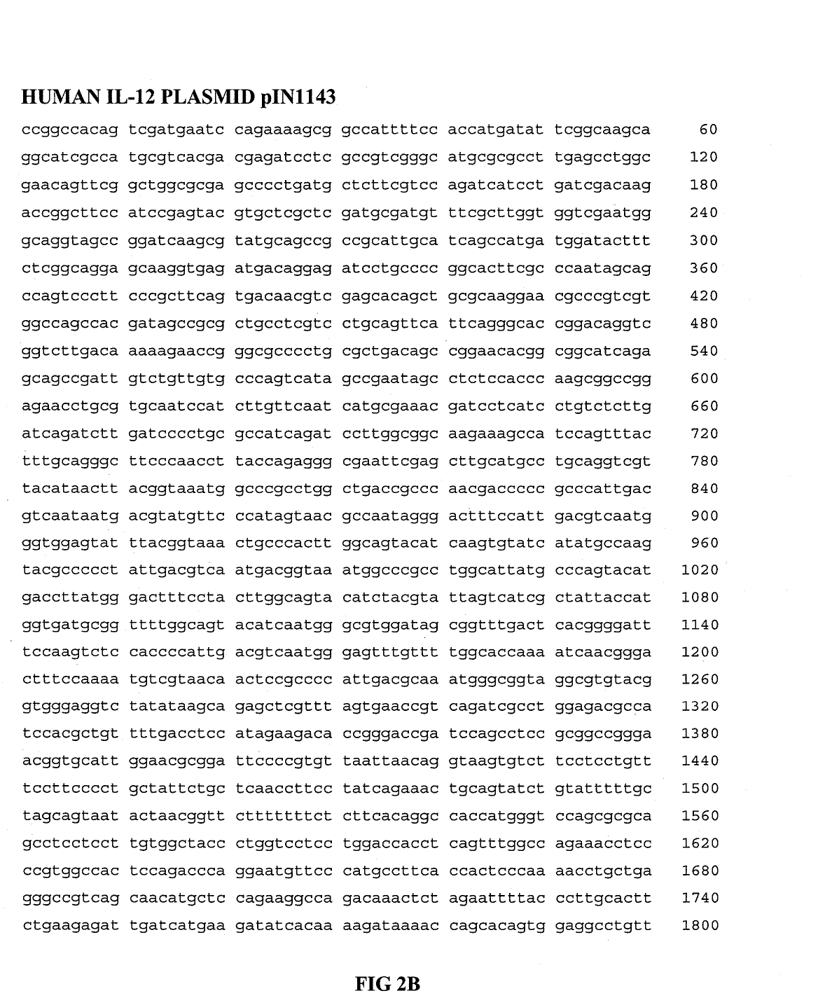

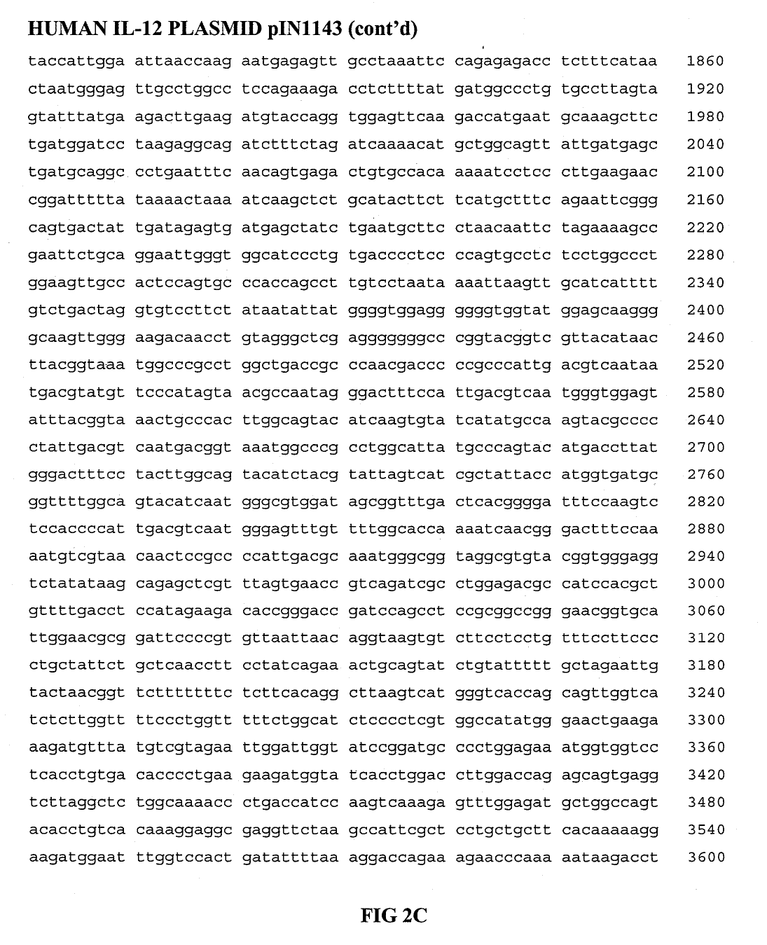

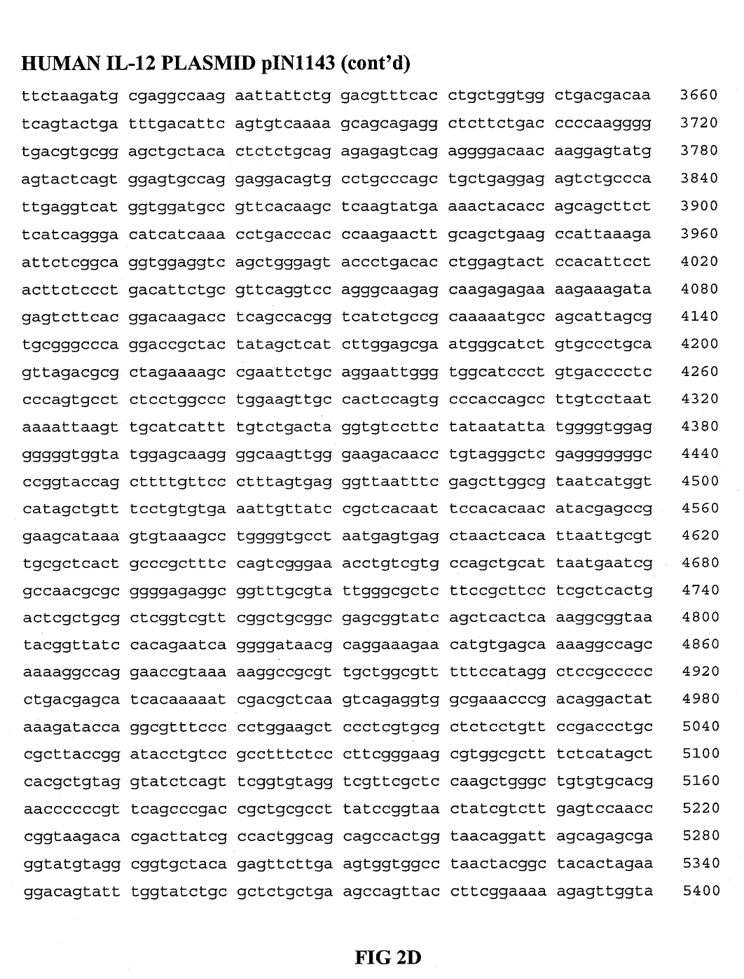

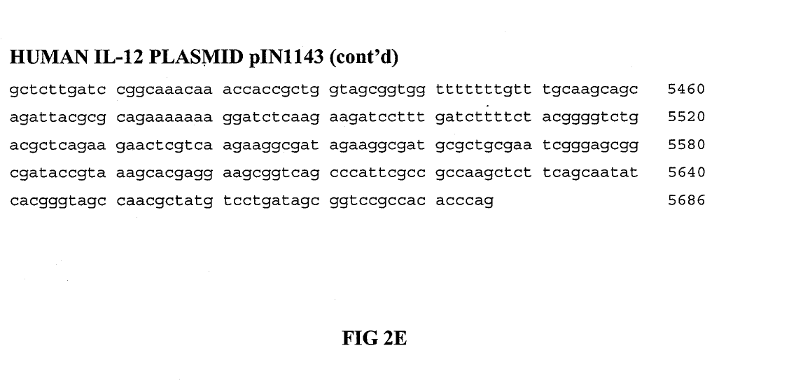

### DETAILED DESCRIPTION OF THE PREFERRED EMBODIMENTS

The present invention relates to gene delivery and gene therapy, and provides novel nucleic acid constructs for expression of interferon alpha in a mammal, formulations for delivery that incorporate a nucleic acid construct for expression, and methods for preparing and using such constructs and formulations. In particular, this invention relates to plasmid constructs for delivery of therapeutic interferon alpha encoding nucleic acids to cells in order to modulate tumor activity, methods of using those constructs (including combination therapy with other agents, such as cytokines, preferably IL-12), as well as methods for preparing such constructs.

I. General

As described, this invention concerns expression systems for the delivery and expression of interferon alpha coding sequences in mammalian cells, and formulations and methods for delivering such expression systems or other expression systems to a mammal.

Therefore, particular genetic constructs are described which includes nucleotide sequences coding for interferon alpha, preferably human interferon alpha. Such a construct can beneficially be formulated and administered as described herein, utilizing the expression systems of this invention.

To allow convenient production of such plasmids, it is generally preferable that the plasmid be capable of replication in a cell to high copy number. Generally such production is carried out in prokaryotic cells, particularly including Esherichia coli (E.coli) cells. Thus, the plasmid preferably contains a replication origin functional in a prokaryotic cell, and preferably the replication origin is one which will direct replication to a high copy number.

It is also possible to utilize synthetic genetic elements in the plasmid constructs.

As described below, these elements affect post-transcriptional processing in eukaryotic systems. Thus, the use of synthetic sequences allows the design of processing characteristics as desired for the particular application. Commonly, the elements will be designed to provide rapid and accurate processing.

For delivery of DNA encoding a desired expression product to a mammalian system, it is usually preferable to utilize a delivery system. Such a system can provide multiple benefits, notably providing stabilization to protect the integrity of the DNA, as well as assisting in cellular uptake.

In addition, the non-DNA components of the formulation may contribute to an immune system enhancement or activation. As a result, components of a delivery system can be selected in conjunction with a particular gene product to enhance or minimize the immuno-stimulatory effect.

The plasmids may also include elements for expression of IL-12 or one or more subunits thereof. Similarly, the treatment may involve administration of an interferon alpha coding sequence and one or more IL-12 coding sequences whether on a single plasmid or on separate plasmids. Such plasmids may be incorporated into compositions for delivery with a protective, interactive non-condensing compound, a cationic lipid and neutral co-lipid, or both.

While these are specific effective examples, other components may be utilized in formulations containing the interferon alpha expression vectors of the present invention to provide effective delivery and expression of interferon alpha or with other coding sequences for which manipulation of the activation of immune system components is desirable.

The selection of delivery system components and preparation methods in conjunction with the selection of coding sequences provides the ability to balance the specific effects of the encoded gene products and the immune system effects of the overall delivery system composition. This capacity to control the biological effects of delivery system formulation administration represents an aspect of the invention in addition to the interferon alpha encoding constructs and specific formulations of delivery systems. Co-selection of the encoded gene product and the delivery system components and parameters provides enhanced desired effects rather than merely providing high level gene expression. In particular, such enhancement is described below for the antitumor effects of the exemplary PVP containing compositions.

For systems in which IL-12 is also administered, the antitumor effect can be greater than merely additive (i.e., greater than merely the sum of the antitumor effects of interferon alpha alone and IL-12 alone). Enhancement of immuno-stimulatory effects is also desirable in other contexts, for example, for vaccine applications.

In contrast, for certain applications, it is preferable to select a delivery systems which minimizes the immune system effects. For example, it is often preferred that the immune system activation be minimized for compositions to be delivered to the lung in order to minimize lung tissue swelling.

a useful approach for selecting the delivery system components and preparation techniques for a particular coding sequence can proceed as follows, but is not limited to these steps or step order.

1. Select a particular genetic construct which provides appropriate expression in vitro.

2. Select delivery system components based on desired immuno-stimulatory effects and/or on in vivo physiological effect. Such effects can be tested or verified in in vivo model systems.

3. Select the other delivery system parameters consistent with the desired immuno-stimulatory effects and/or consistent with enhancing the desired in vivo physiological effect. Such parameters can, for example, include the amount and ratio of DNA to one or more other composition components, the relative amounts of non-DNA composition components, the size of delivery system formulation particles, the percent supercoiled DNA for circular dsDNA vectors, and the specific method of preparation of delivery system formulation particles. The particular parameters relevant for specific types of formulations will be apparent or readily determined by testing.

The description below illustrates the selection of components and parameters in the context of interferon alpha encoding constructs. However, it should be recognized that the approach is applicable to constructs containing a variety of other coding sequences.

II. Plasmid Construct Expression Systems

A. Plasmid Design and Construction

For the methods and constructs of this invention, a number of different plasmids were constructed which are useful for delivery and expression of sequences encoding interferon alpha. Thus, these plasmids contain coding regions for interferon alpha, along with genetic elements necessary or useful for expression of those coding regions.

While these embodiments utilized interferon alpha cDNA clones or partial genomic sequences from a particular source, those skilled in the art could readily obtain interferon alpha coding sequences from other sources, or obtain a coding sequence by identifying a cDNA clone in a library using a probe(s) based on the published interferon alpha coding and/or flanking sequences. This also applies to the IL-12 coding sequences utilized in the embodiments described herein.

Coding sequences for interferon alpha were incorporated into an expression plasmid that contains eukaryotic and bacterial genetic elements. Eukaryotic genetic elements include the CMV immediate early promoter and 5′ UTR, and a human growth hormone 3′ UTR/poly(a) signal, which influence gene expression by controlling the accuracy and efficiency of RNA processing, mRNA stability, and translation.

The human growth hormone 3′ UTR is from a human growth hormone gene, and preferably includes a poly(a) signal. This sequence can be linked immediately following the natural translation termination codon for a cDNA sequence, genomic sequence, modified genomic sequence, or synthetic sequence coding for interferon alpha.

An example of a human growth hormone 3′ UTR/poly(a) signal is shown by the human growth hormone 3′ UTR (nucleotides 1-100) and 3′ flanking sequence (nucleotides 101-191; GenBank accession #J03071, HUMGHCSA) below. (SEQ ID NO:13)

```
    1
GGGTGGCATCCCTGTGACCCCTCCCCAGTGCCTCTCCTGGCCCTGGAAGT
                                                        Poly (a)signal
  51
TGCCACTCCAGTGCCCACCAGCCTTGTCCTAATAAAATTAAGTTGCATCA
101
TTTTGTCTGACTAGGTGTCCTTCTATAATATTATGGGGTGGAGGGGGGTG
151
GTATGGAGCAAGGGGCAAGTTGGGAAGACAACCTGTAGGGC
```

The 5′ and 3′ UTR and flanking regions can be further and more precisely defined by routine methodology, e.g., deletion or mutation analysis or their equivalents., and can be modified to provide other sequences having appropriate transcriptional and translational functions.

1. Construction of plasmid: Plasmid Backbone, human interferon alpha cDNA, Final Construct

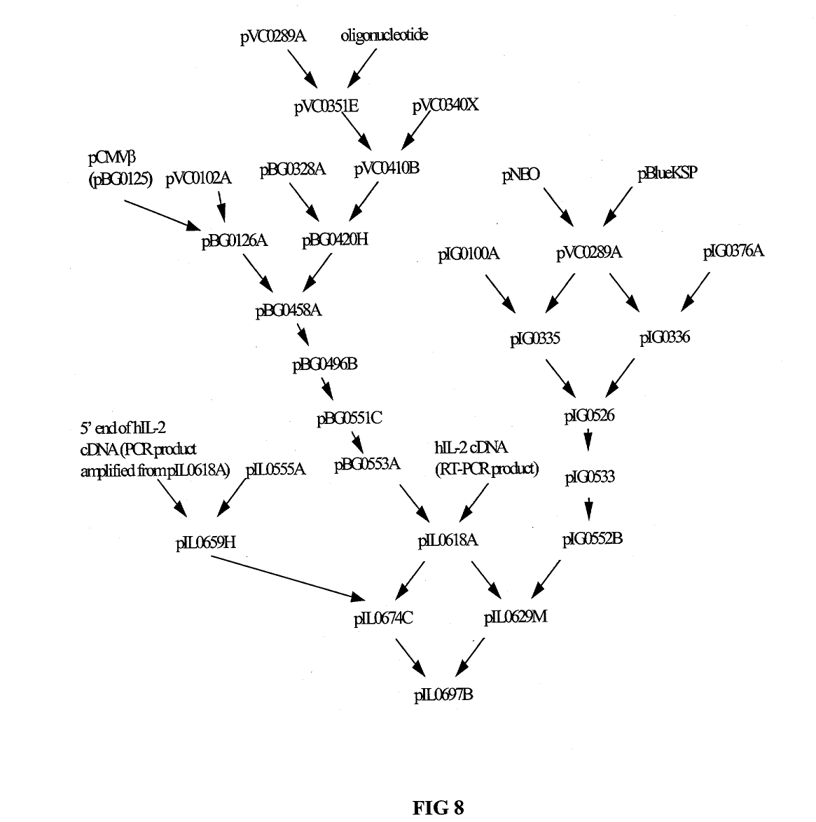

a diagrammatic representation of the PCR products and plasmids involved in creation of an exemplary construct is shown below in FIG. 8.

Plasmid pIF0921 was constructed from commercially available plasmids, and contains the TN5 gene encoding the kanamycin resistance gene, the pUC origin of replication, the CMV enhancer and promoter to base +112, a synthetic intron called IVS8, the human IFN-a 2b gene, and the human growth hormone 3′ UTR. The plasmid construction descendancy for pIL0697 is shown in FIG. 8. pIL0697 was cut with BamHI and Xba I and the hIFN-a2b PCR product, which had been amplified from human genomic DNA with BamHI and Xba I ends, was cloned into the pIL0697 backbone in place of the IL-2 coding region. The resulting plasmid was pIF0863. pIF0863 was cut with Nco I and intron IVS8 from pCT0828 was cloned in. The resulting plasmid was pIF0890. pIF0890 was cut with Nde I and Pac I and an additional region of the CMV 5′ UTR to base +112 was cloned in from plasmid pLC0888.

B. Synthetic Genetic Elements

In some embodiments, some or all of the genetic elements can be synthetic, derived from synthetic oligonucleotides, and thus are not obtained directly from natural genetic sequences. These synthetic elements are appropriate for use in many different expression vectors.

A synthetic intron is designed with splice sites that ensure that RNA splicing is accurate and efficient. A synthetic 3′ UTR/poly(A) signal is designed to facilitate mRNA 3′ end formation and mRNA stability. A synthetic 5′ UTR is designed to facilitate the initiation of translation. The design of exemplary synthetic elements is described in more detail below.

1. Summary of Synthetic Element Features

Exemplary synthetic 5′UTR, intron, and 3′UTR/poly(A) signal have the general features shown below:

| | |
|---|---|
| 5′ UTR | Short. |
| | Lack of secondary structure. |
| | Kozak sequence. |
| | Site for intron insertion. |
| Intron | 5′ splice site sequence matches consensus. |
| | 5′ splice site sequence is exactly |
| | complementary to 5′ end of U1 snRNA. |
| | Branch point sequence matches consensus. |
| | Branch point sequence is complementary to U2 |
| | snRNA. |
| | 3′ splice site matches consensus. |
| | Polypyrimidine tract is 16 bases in length and |
| | contains 7 consecutive T's. |
| | (The tract preferably contains at least 5 |
| | consecutive T's.) |
| | Contains internal restriction enzyme sites. |
| | BbsI cleaves at the 5′ss, EarI cleaves at the |
| | 3′ss. |
| 3′ UTR/Poly(A) | Based on rabbit β-globin 3′ UTR/poly(A) |
| | signal. |
| | Consists of two poly(A) signals in tandem. |

2. Features of the Synthetic 5′UTR (UT6):

The 5′ untranslated region (5′UTR) influences the translational efficiency of messenger RNA, and is therefore an important determinant of eukaryotic gene expression. The synthetic 5′UTR sequence (UT6) has been designed to maximize the translational efficiency of mRNAs encoded by vectors that express genes of therapeutic interest.

The sequence of the synthetic 5′ UTR (UT6) is shown below. The Kozak sequence is in boldface and the initiation codon is double underlined. The location of the intron (between residues 48 and 49) is indicated by the filled triangle and the sequences that form the exonic portion of consensus splice sites are single underlined. The restriction sites for HindIII and NcoI are overlined. (SEQ ID NO:14)

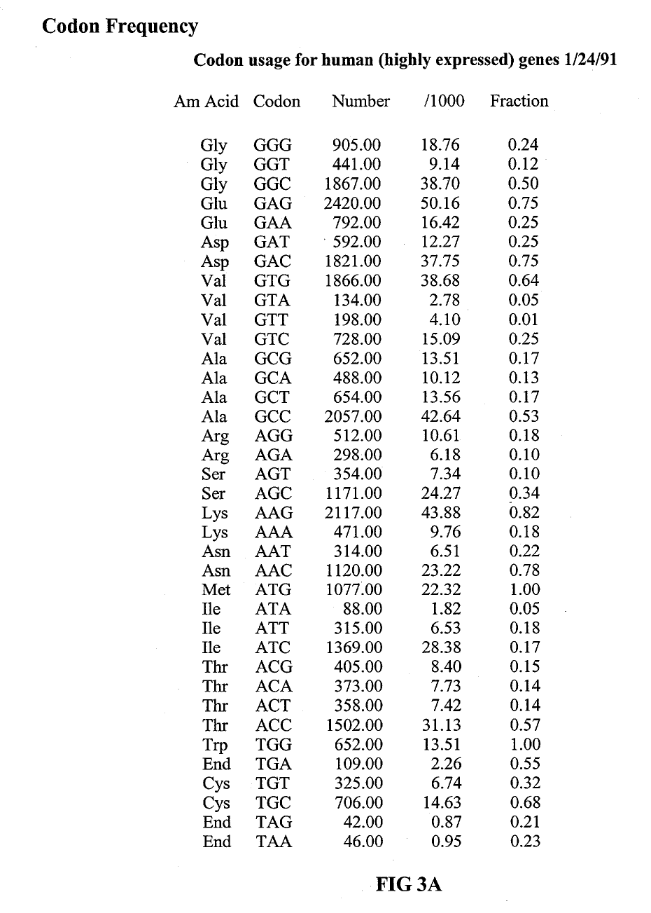

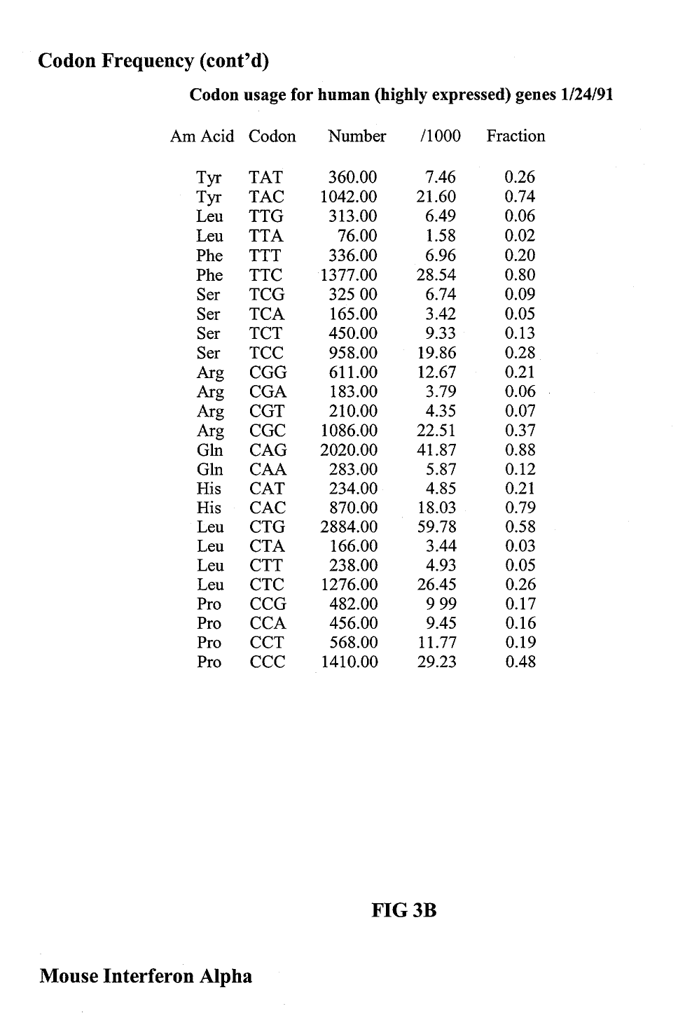

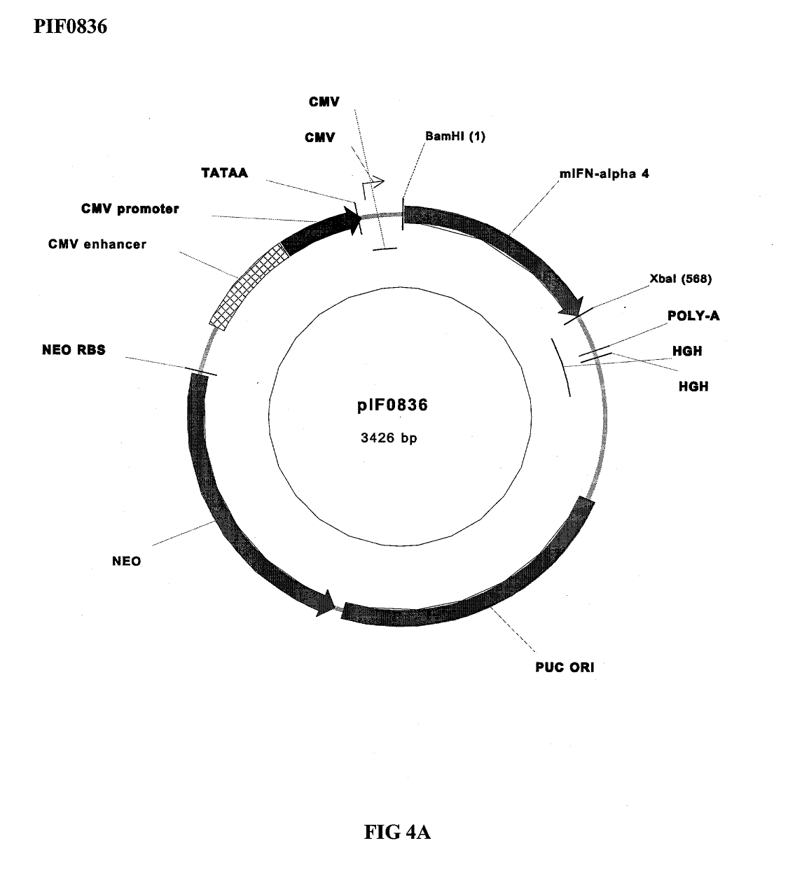

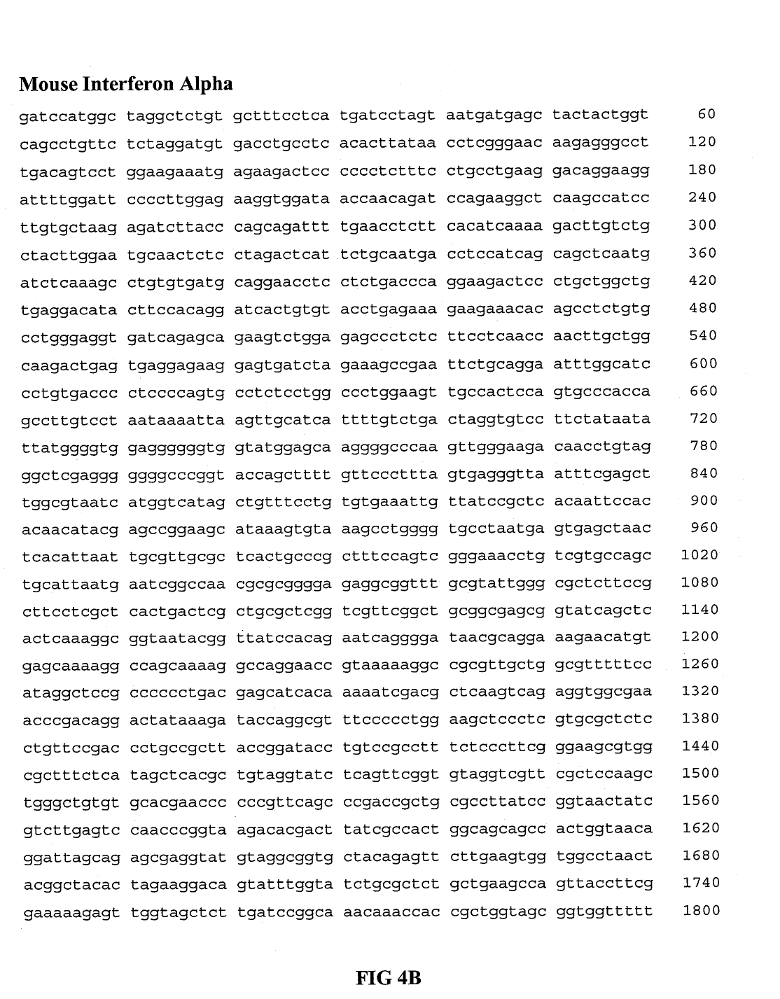

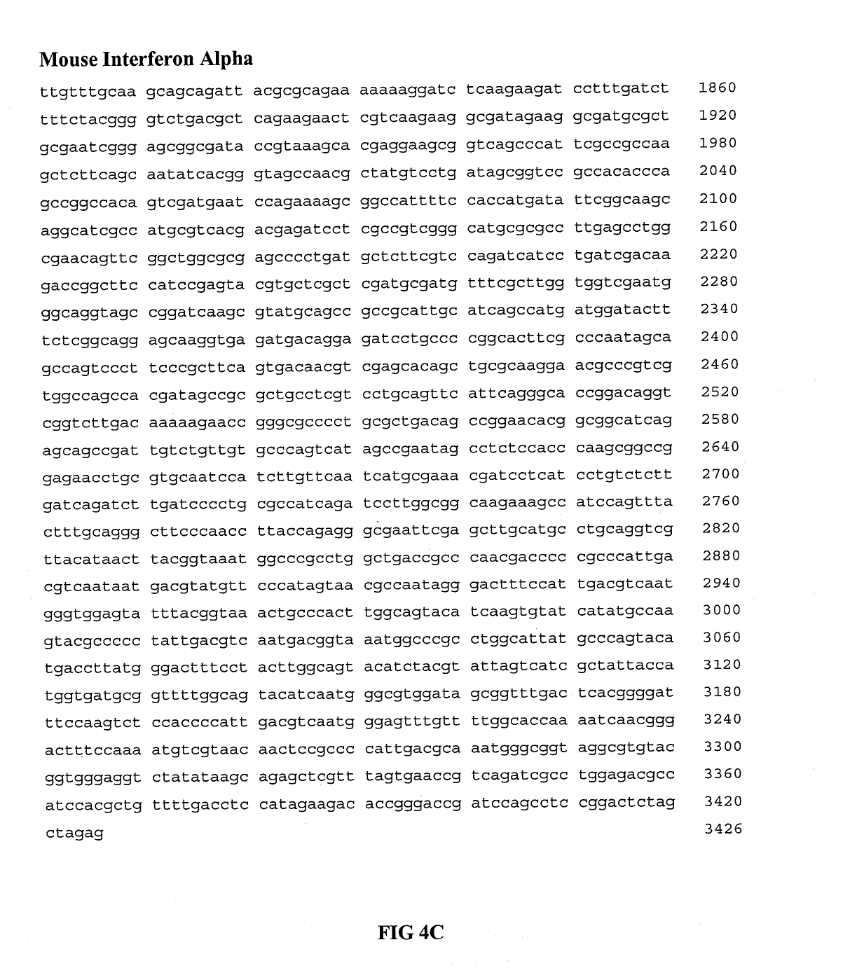

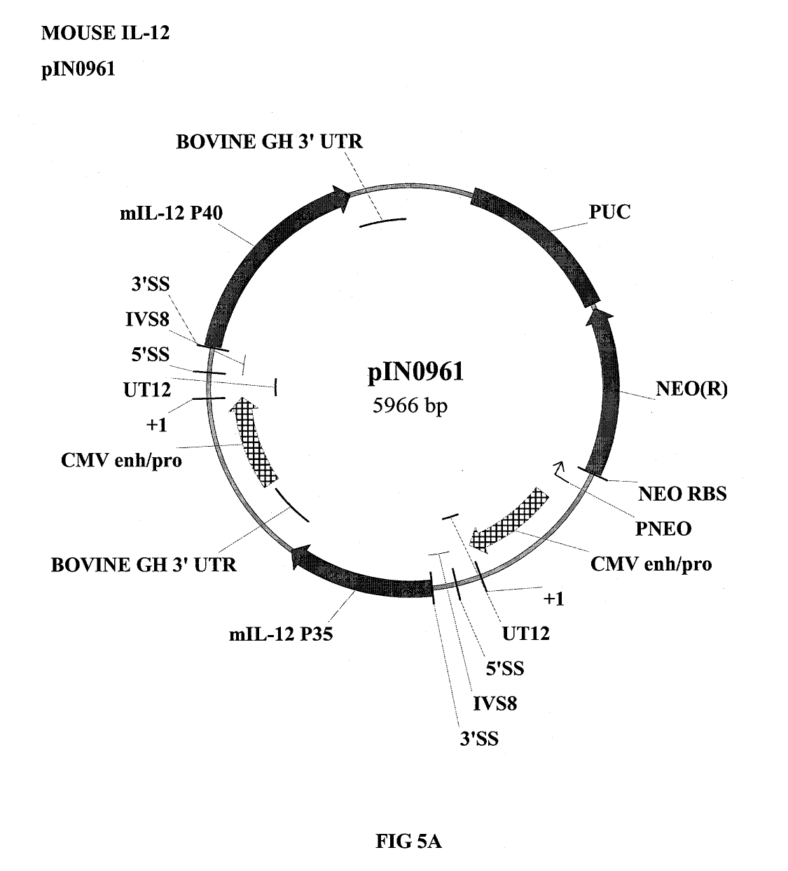

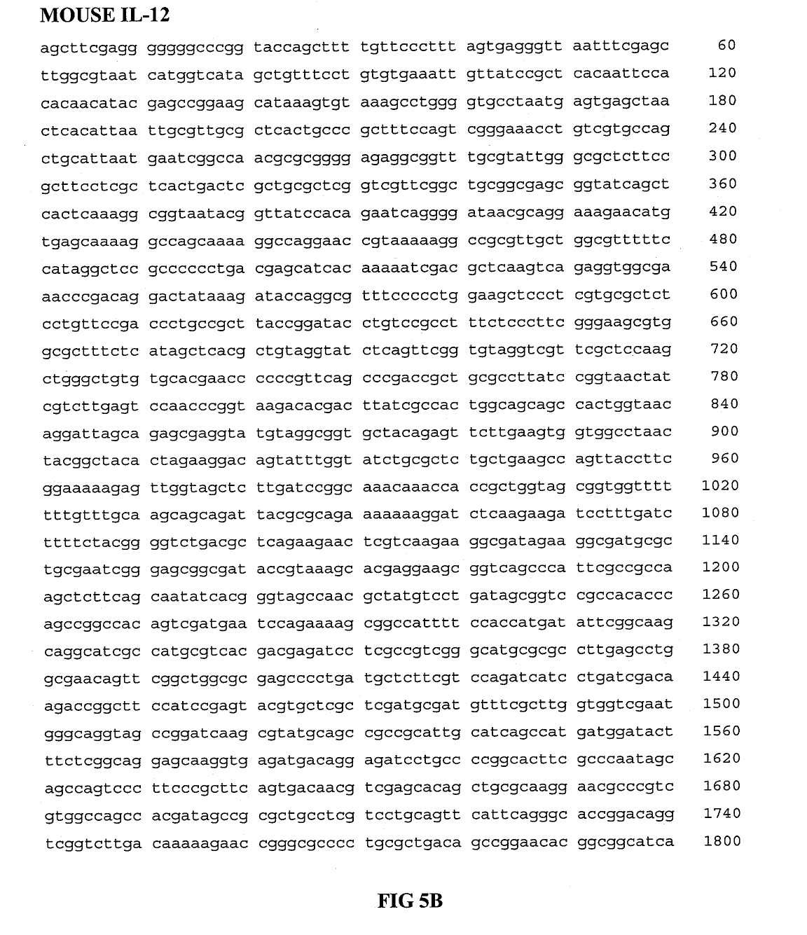

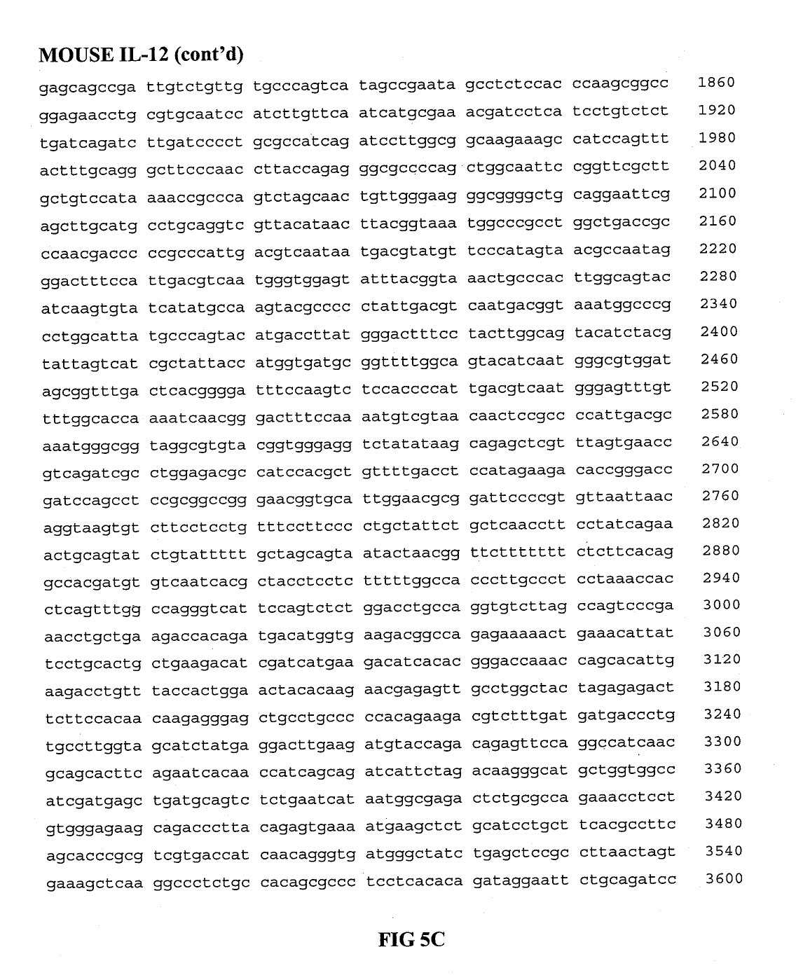

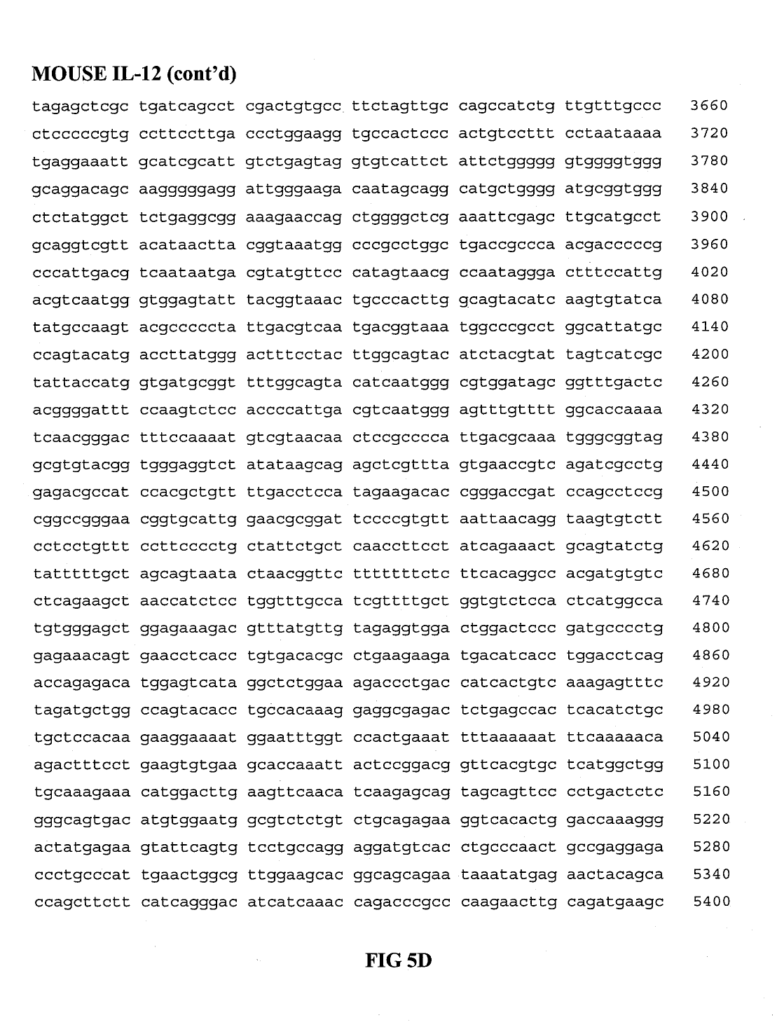

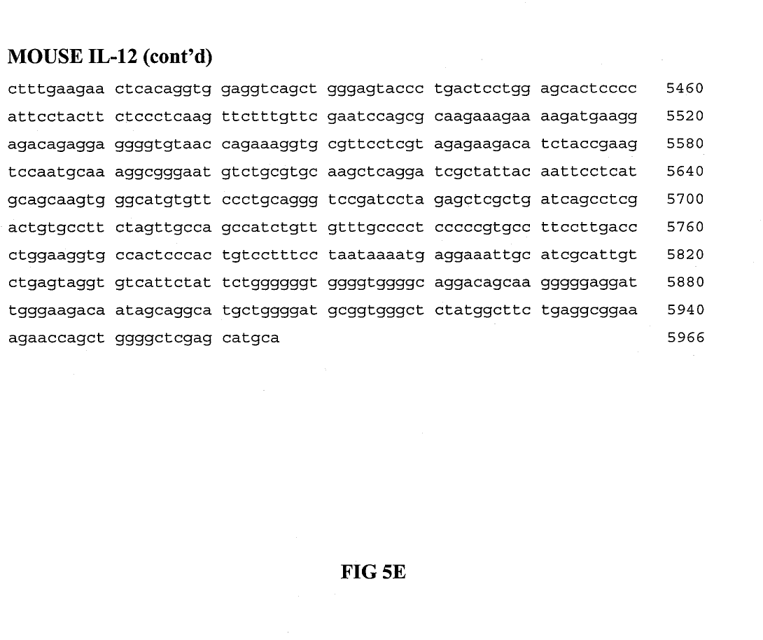

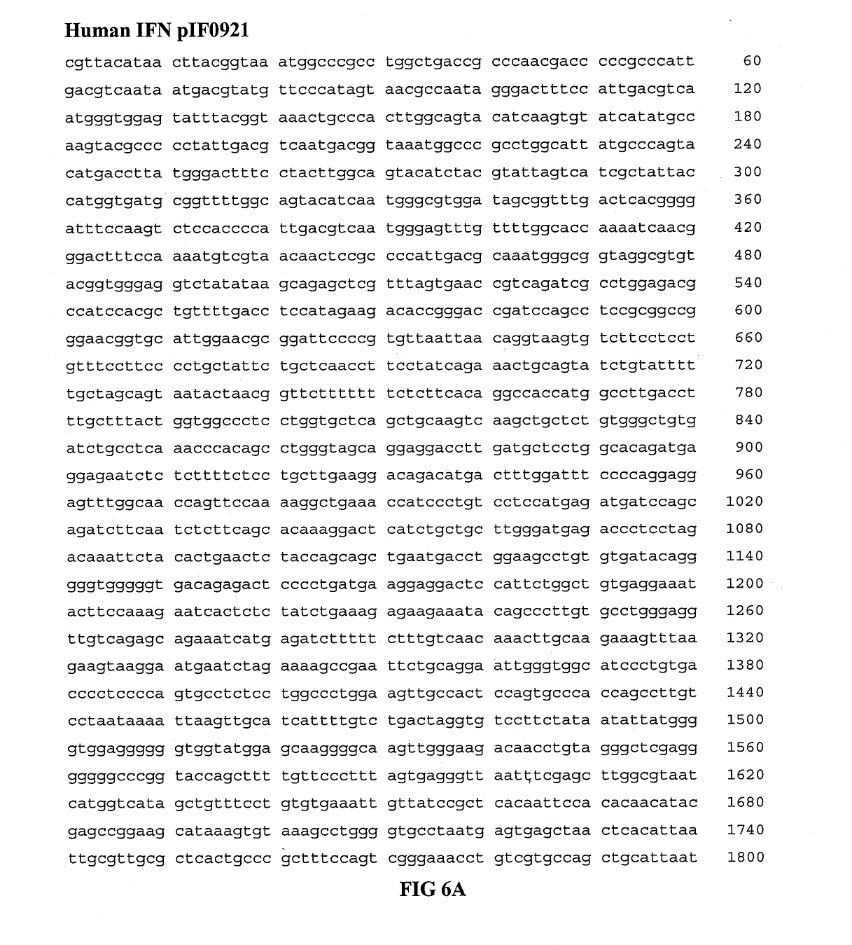

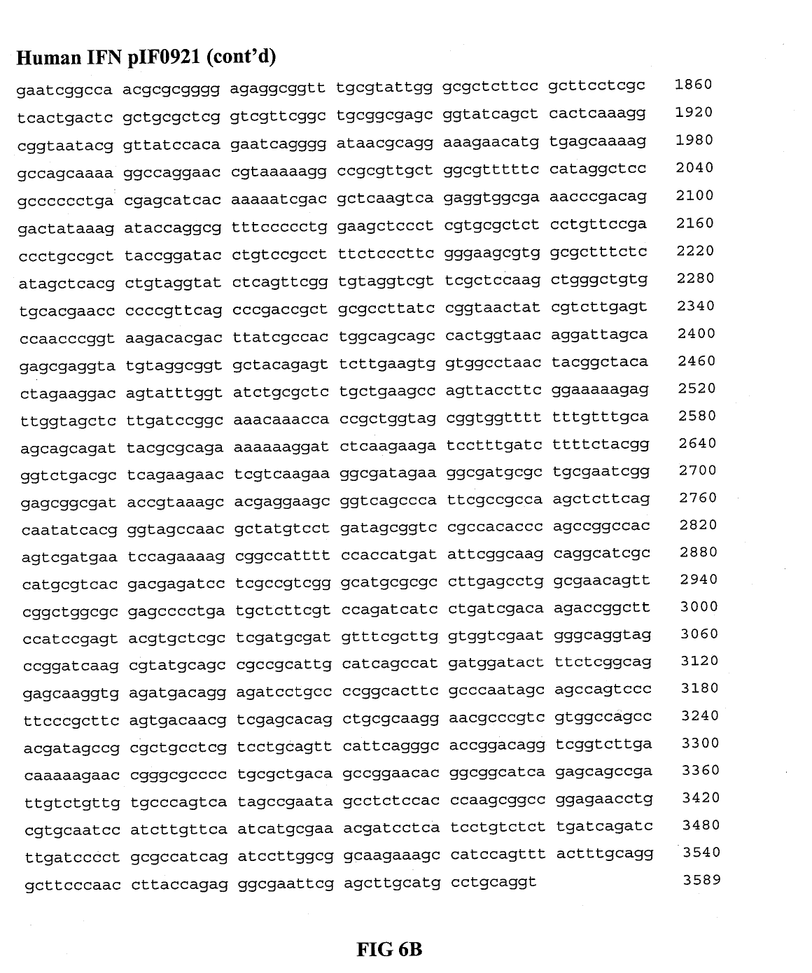

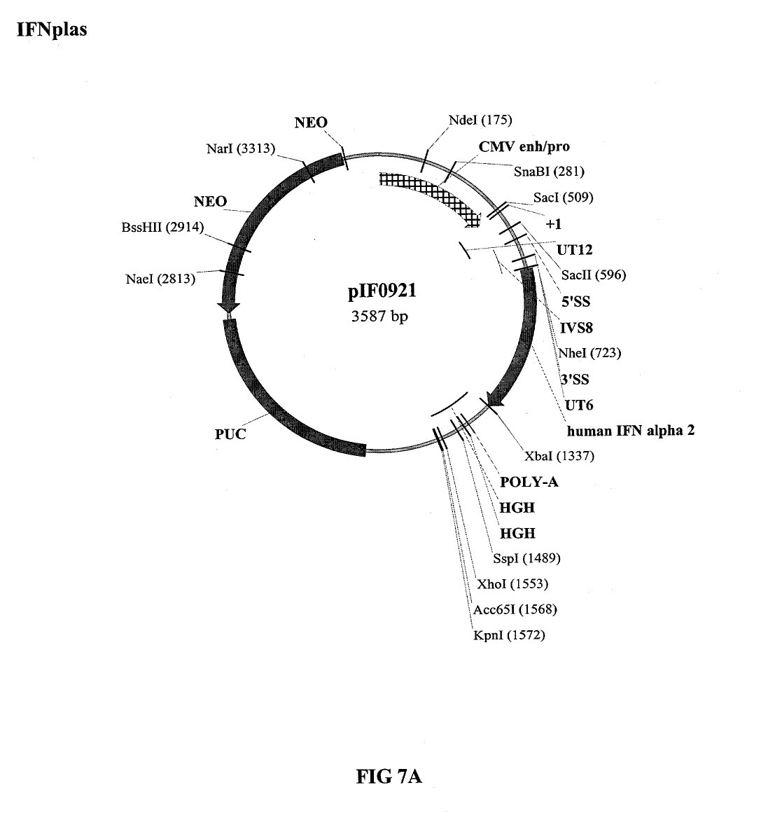

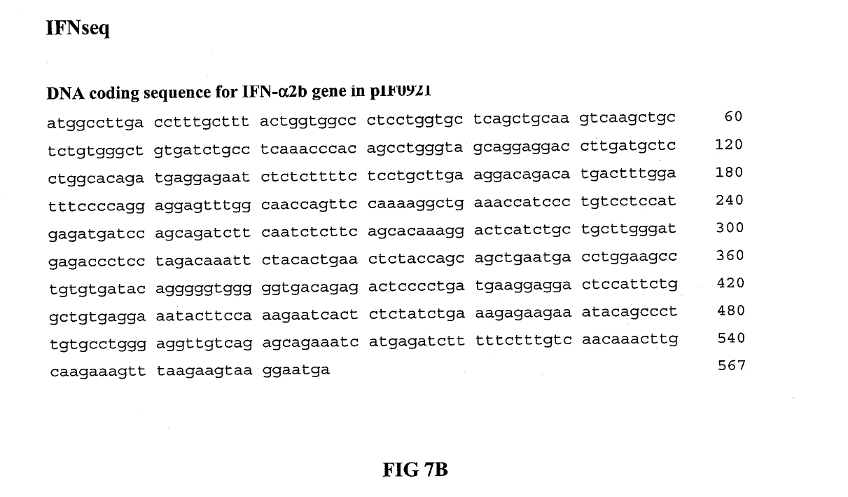

``HindIII``
AAGCTTACTCAACACAATAACAAACTTACTTACAATCTTAATTAACAGGC
````NcoI```
CACCATGG

The 5′ untranslated region (5′ UTR), located between the cap site and initiation codon, is known to influence the efficiency of mRNA translation. Any features that influence the accessibility of the 5′ cap structure to initiation factors, the binding and subsequent migration of the 43S preinitiation complex, or the recognition of the initiation codon, will influence mRNA translatability. An efficient 5′ UTR is expected to be one that is moderate in length, devoid of secondary structure, devoid of upstream initiation codons, and has an AUG within an optimal local context (Kozak, 1994, Biochimie 76:815-821; Jansen et al., 1994). A 5′ UTR with these characteristics should allow efficient recognition of the 5′ cap structure, followed by rapid and unimpeded ribosome scanning by the ribosome, thereby facilitating the translation initiation process.

The sequence of the synthetic 5′UTR was designed to be moderate in length (54 nucleotides (nts)), to be deficient in G but rich in C and A residues, to lack an upstream ATG, to place the intended ATG within the context of a optimal Kozak sequence (CCACCATGG), and to lack potential secondary structure. The synthetic 5′ UTR sequence was also designed to lack AU-rich sequences that target mRNAs to be rapidly degraded in the cytoplasm.

Experiments have demonstrated that introns increase gene expression from cDNA vectors, and that introns located in the 5′ UTR are more effective than ones located in the 3′ UTR (Huang and Gorman, 1990, Mol. Cell. Biol. 10:1805-1810; Evans and Scarpulla, 1989, Gene 84:135-142; Brinster et al., 1988, Proc. Natl. Acad. Sci. USA 85:836-840; Palmiter et al., 1991, Proc. Natl. Acad. Sci. USA 88:478-482; Choi et al., 1991, Mol. Cell. Biol. 11:3070-3074). Accordingly, the synthetic 5′ UTR sequence was designed to accommodate an intron with consensus splice site sequences. The intron may, for example, be located between residues 48 and 49 (See intron sequence structure below). The CAG at position 46-48 is the exonic portion of a consensus 5′ splice site. The G at position 49 is the exonic portion of a consensus 3′ splice site.

To facilitate cloning manipulations, the synthetic 5′ UTR sequence was designed to begin with a HindIII site and terminate with a NcoI site.

3. Features of the Synthetic Intron

RNA splicing is required for the expression of most eukaryotic genes. For optimal gene expression, RNA splicing must be highly efficient and accurate. A synthetic intron, termed OPTIVS8B, was designed to be maximally efficient and accurate.

The structure of the exemplary synthetic intron, OPTIVS8 is shown below. Sequences for the 5′ splice site (5′ss), branch point (bp), and 3′ splice site (3′ss) are double underlined. The recognition sequences for the restriction enzymes BbsI and Earl are overlined. The cleavage site for BbsI corresponds to the 5′ss, and the cleavage site for Earl corresponds to the 3′ss.

```
        5′ss bp 3′ss
          | BbsI | EarI |
5′CAG GTAAGTGTCTTC---(77)---TACTAACGGTTCTTTTTTTCTCTTCACAG G 3′
(SEQ ID NO.15) (SEQ ID NO.16)
```

The 5′ splice site (5′ss) sequence matches the established consensus sequence, MAG 9 GTRAGT, where M=C or A, and R=G or A. Since the mechanism of splicing involves an interaction between the 5′ss of the pre-mRNA and U1 snRNA, the 5′ss sequence of OPTIVS8B was chosen to be exactly complementary to the 5′ end of U1 snRNA.

| | |
|---|---|
| 5′ss U1 RNA | 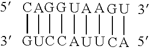 |
| BP U2 RNA | 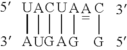 |

The sequence of the 3′ splice site (3′ss) matches the established consensus sequence, Y₁₁NYAG 9 G, where Y=C or T, and N=any base. In 3′ splice sites, the polypyrimidine tract (Y₁₁) is the major determinant of splice site strength. For optimal splice site function in OPTIVS8B, the length of the polypyrimidine tract was extended to 16 bases, and its sequence was adjusted to contain 7 consecutive T residues. This feature was included because Roscigno et al. (1993) demonstrated that optimal splicing requires the presence of at least 5 consecutive T residues in the polypyrimidine tract.

Splicing in vitro is generally optimal when introns are >80 nts in length (Wieringa, et al., 1984; Ulfendahl et al., 1985, Nucl. Acids Res. 13:6299-6315). Although many introns may be thousands of bases in length, most naturally occurring introns are 90-200 nt in length (Hawkins, 1988, Nucl. Acids Res. 16:9893-9908). The length of the synthetic intron (118 nts) falls within this latter range.

OPTIVS8B was designed with three internal restriction enzyme sites, BbsI, NheI, and Earl. These restriction sites facilitate the screening and identification of genes that contain the synthetic intron sequence. In addition, the BbsI and Earl sites were placed so that their cleavage sites exactly correspond to the 5′ss (BbsI) or 3′ss (Earl). The sequence of the polypyrimidine tract was specifically designed to accommodate the Earl restriction site. Inclusion of the BbsI and Earl sites at these locations is useful because they permit the intron to be precisely deleted from a gene. They also permit the generation of an “intron cassette” that can be inserted at other locations within a gene.

The 77 bases between the BbsI site and the branch point sequence are random in sequence, except for the inclusion of the NheI restriction site.

4. Features of the Synthetic 3′ UTR/poly(A) Signal:

The 3′ ends of eukaryotic mRNAs are formed by the process of polyadenylation. This process involves site specific site RNA cleavage, followed by addition of a poly(A) tail. RNAs that lack a poly(A) tail are highly unstable. Thus, the efficiency of cleavage/polyadenylation is a major determinant of mRNA levels, and thereby, of gene expression levels. 2XPA1 is a synthetic sequence, containing two efficient poly(A) signals, that is designed to be maximally effective in polyadenylation.

A poly(A) signal is required for the formation of the 3′ end of most eukaryotic mRNA. The signal directs two RNA processing reactions: site-specific endonucleolytic cleavage of the RNA transcript, and stepwise addition of adenylates (approximately 250) to the newly generated 3′ end to form the poly(A) tail. A poly(A) signal has three parts: hexanucleotide, cleavage site, and downstream element. The hexanucleotide is typically AAUAAA and cleavage sites are most frequently 3′ to the dinucleotide CA (Sheets et al., 1987). Downstream elements are required for optimal poly(A) signal function and are located downstream of the cleavage site. The sequence requirement for downstream elements is not yet fully established, but is generally viewed as UG- or U-rich sequences (Wickens, 1990; Proudfoot, 1991, Cell 64:671-674; Wahle, 1992, Bioessays 14:113-118; Chen and Nordstrom, 1992, Nucl. Acids Res. 20:2565-2572).

Naturally occurring poly(A) signals are highly variable in their effectiveness (Peterson, 1992). The effectiveness of a particular poly(A) signal is mostly determined by the quality of the downstream element. (Wahle, 1992). In expression vectors designed to express genes of therapeutic interest, it is important to have a poly(A) signal that is as efficient as possible.

Poly(A) efficiency is important for gene expression, because transcripts that fail to be cleaved and polyadenylated are rapidly degraded in the nuclear compartment. In fact, the efficiency of polyadenylation in living cells is difficult to measure, since nonpolyadenylated RNAs are so unstable. In addition to being required for mRNA stability, poly(A) tails contribute to the translatability of mRNA, and may influence other RNA processing reactions such as splicing or RNA transport ((Jackson and Standart, 1990, Cell 62:15-24; Wahle, 1992).

Some eukaryotic genes have more than one poly(A) site, implying that if the cleavage/polyadenylation reaction fails to occur at the first site, it will occur at one of the later sites. In COS cell transfection experiments, a gene with two strong poly(A) sites yielded approximately two-fold more mRNA than one with a single strong poly(A) site (Bordonaro, 1995). These data suggest that a significant fraction of transcripts remain unprocessed even with a single “efficient” poly(A) signal. Thus, it may be preferable to include more than one poly(A) site.

The sequence of the exemplary synthetic poly(A) signal is shown below. The sequence is named 2XPA. The hexanucleotide sequences and downstream element sequences are double underlined, and the two poly(A) sites are labeled as pA#1 and pA#2. Convenient restriction sites are overlined. The entire 2XPA unit may be transferred in cloning experiments as a XbaI-KpnI fragment. Deletion of the internal BspHI fragment results in the formation of a 1XPA unit. (SEQ ID NO.17)

```
  XbaI BspHI
TCTAGAGCATTTTTCCCTCTGCCAAAAATTATGGGGACATCATGAAGCCCCTTGAGCATCTGACG
                                                              pA#1
                Hex | Downstream element
TCTGGCTAATAAAGGAAATTTATTTTCATTGCAATAGTGTGTTGGAATTTTTTGTGTCTCTCACT
                                                                                      +E,usn BspHI
CGGTACTAGAGCATTTTTCCCTCTGCCAAAAATTATGGGGACATCATGAAGCCCCTTGAGCATCT
                                                                      pA#2
                        Hex | Downstream element
GACGTCTGGCTAATAAAGGAAATTTATTTTCATTGCAATAGTGTGTTGGAATTTTTTGTGTCTCT
            KpnI
CACTCGGTACC
```

The sequence of the synthetic poly(A) site shown above is based on the sequence of the rabbit β-globin poly(A) signal, a signal that has been characterized in the literature as strong (Gil and Proudfoot, 1987, Cell 49:399-406; Gil and Proudfoot, 1984, Nature 312:473-474). One of its key features is the structure of its downstream element, which contains both UG- and U-rich domains.

A double-stranded DNA sequence corresponding to the 1XPA sequence was constructed from synthetic oligonucleotides. Two copies of the 1XPA sequence were then joined to form the 2XPA sequence. The sequences were joined in such as way as to have a unique XbaI site at the 5′ end of the first poly(A) signal containing fragment, and a unique KpnI site at the 3′ end of the second poly(A) signal containing fragment.

C. Interferon Alpha and IL-12 Coding Sequences

The nucleotide sequence of a natural human interferon alpha coding sequences is known, and is provided below, along with a synthetic sequence which also codes for human interferon alpha. The same applies with respect to the IL-12 coding sequences.

In some cases, instead of the natural sequence coding for interferon alpha, it is advantageous to utilize synthetic sequences which encode interferon alpha. Such synthetic sequences have alternate codon usage from the natural sequence, and thus have dramatically different nucleotide sequences from the natural sequence. In particular, synthetic sequences can be used which have codon usage at least partially optimized for expression in a human. The natural sequences do not have such optimal codon usage. Preferably, substantially all the codons are optimized.

Optimal codon usage in humans is indicated by codon usage frequencies for highly expressed human genes, as shown in FIGS. 3A-B. The codon usage chart is from the program “Human_High.cod” from the Wisconsin Sequence Analysis Package, Version 8.1, Genetics Computer Group, Madison, Wis. The codons which are most frequently used in highly expressed human genes are presumptively the optimal codons for expression in human host cells, and thus form the basis for constructing a synthetic coding sequence. An example of a synthetic interferon alpha coding sequence is shown as the bottom sequence in the table below.

However, rather than a sequence having fully optimized codon usage, it may be desirable to utilize an interferon alpha encoding sequence which has optimized codon usage except in areas where the same amino acid is too close together or abundant to make uniform codon usage optimal.

In addition, other synthetic sequences can be used which have substantial portions of the codon usage optimized, for example, with at least 50%, 70%, 80% or 90% optimized codons as compared to a natural coding sequence. Other particular synthetic sequences for interferon alpha can be selected by reference to the codon usage chart in FIGS. 3A-B. A sequence is selected by choosing a codon for each of the amino acids of the polypeptide sequences. DNA molecules corresponding to each of the polypeptides can then by constructed by routine chemical synthesis methods. For example, shorter oligonucleotides can be synthesized, and then ligated in the appropriate relationships to construct the full-length coding sequences.

The following sequences are provided in the sequence listing herein: interferon alpha amino acid sequence, SEQ ID NO:9; interferon alpha wild type nucleic acid sequence, SEQ ID NO:10; interferon alpha synthetic nucleic acid sequence with optimized codon usage, SEQ ID NO:11; interferon alpha nucleic acid sequence with additional/semi-optimized codon usage, SEQ ID NO:12; IL-12 p40 subunit amino acid sequence, SEQ ID NO:1; IL-12 p40 wild type nucleic acid sequence, SEQ ID NO:2; IL-12 p40 synthetic nucleic acid sequence with all codons optimized, SEQ ID NO:3; IL-12 p40 subunit nucleic acid sequence with all codons optimized except when same nucleic acids were too close/abundant, SEQ ID NO:4; IL-12 p35 amino acid sequence, SEQ ID NO:5; IL-12 p35 wild type nucleic acid sequence, SEQ ID NO:6; IL-12 p35 synthetic nucleic acid sequence with all codons optimized, SEQ ID NO:7; IL-12 p35 subunit nucleic acid sequence with all codons optimized except when same nucleic acids were too close/abundant, SEQ ID NO:8. Those skilled in the art will realize that various nucleic acid sequences with optimized codon usage can be constructed, for example based on the various combinations shown below, wherein optimal usage for each codon is shown below the IL-12 p35 and p40 subunit wild type sequences and the interferon alpha wild type sequence.

### Sequences Encoding Human IL-12 p35

First line: natural sequence (SEQ ID NO. 6)

Second line: all codons optimized (SEQ ID NO. 7)

Third line: all codons optimized except when same nucleic acids were too close/abundant (changes between second and third lines bolded) (SEQ ID NO. 8)

ATG TGT CCA GCG CGC AGC CTC CTC CTT GTG GCT ACC CTG GTC CTC CTG GAC CAC CTC ACT
ATG TGC CCC GCC CGC AGC CTG CTG CTG GTG GCC ACC CTG GTG CTG CTG GAC CAC CTG AGC
ATG TGC CCC GCC CGC AGC CTG CTG CTC GTG GCC ACC CTG GTG CTC CTG GAC CAC CTC AGC
TTG GCC AGA AAC CTC CCC GTG GCC ACT CCA GAC CCA GGA ATG TTC CCA TGC CTT CAC CAC
CTG GCC CGC AAC CTG CCC GTG GCC ACC CCC GAC CCC GGC ATG TTC CCC TGC CTG CAC CAC
CTG GCC CGC AAC CTC CCC GTG GCC ACC CCA GAC CCC GGC ATG TTC CCA TGC CTG CAC CAC
TCC CAA AAC CTG CTG AGG GCC GTC AGC AAC ATG CTC CAG AAG GCC AGA CAA ACT CTA GAA
AGC CAG AAC CTG CTG GCG GCC GTG AGC AAC ATG CTG CAG AAG GCC GCG CAG ACC CTG GAG
AGC CAG AAC CTG CTG GCG GCC GTG AGC AAC ATG CTG CAG AAG GCC GCG CAG ACC CTG GAG
TTT TAC CCT TGC ACT TCT GAA GAG ATT GAT CAT GAA GAT ATC ACA AAA GAT AAA ACC AGC
TTC TAC CCC TGC ACC AGC GAG GAG ATC GAC CAC GAG GAC ATC ACC AAG GAC AAG ACC AGC
TTC TAC CCC TGC ACC AGC GAG GAG ATC GAC CAC GAG GAC ATC ACC AAG GAC AAG ACC AGC
ACA GTG GAG GCC TGT TTA CCA TTG GAA TTA ACC AAG AAT GAG AGT TGC CTA AAT TCC AGA
ACC GTG GAG GCC TGC CTG CCC CTG GAG CTG ACC AAG AAC GAG AGC TGC CTG AAC AGC CGC
ACC GTG GAG GCC TGC CTG CCC CTC GAG TTA ACC AAG AAC GAG AGC TGC CTC AAC AGC CGC
GAG ACC TCT TTC ATA ACT AAT GGG AGT TGC CTG GCC TCC AGA AAG ACC TCT TTT ATG ATG
GAG ACC AGC TTC ATC ACC AAC GGC AGC TGC CTG GCC AGC CGC AAG ACC AGC TTC ATG ATG
GAG ACC TCC TTC ATC ACC AAC GGC ACT TGC CTG GCC TCC CGC AAG ACC AGC TTC ATG ATG
GCC CTG TGC CTT AGT AGT ATT TAT GAA GAC TTG AAG ATG TAC CAG GTG GAG TTC AAG ACC
GCC CTG TGC CTG AGC AGC ATC TAC GAG GAC CTG AAG ATG TAC CAG GTG GAG TTC AAG ACC
GCC CTG TGC CTG AGC TCC ATC TAC GAG GAC CTG AAG ATG TAC CAG GTG GAG TTC AAG ACC
ATG AAT GCA AAG CTT CTG ATG GAT CCT AAG AGG CAG ATC TTT CTA GAT CAA AAC ATG CTG
ATG AAC GCC AAG CTG CTG ATG GAC CCC AAG CTC CAG ATC TTC CTG GAC CAG AAC ATG CTG
ATG AAC GCC AAG CTC CTG ATG GAC CCC AAG CTC CAG ATC TTC CTG GAC CAG AAC ATG CTG
GCA GTT ATT GAT GAG CTG ATG CAG GCC CTG AAT TTC AAC AGT GAG ACT GTG CCA CAA AAA
GCC GTG ATC GAC GAG CTG ATG CAG GCC CTG AAC TTC AAC AGC GAG ACC GTG CCC CAG AAG
GCC GTG ATC GAC GAG CTG ATG CAG GCC CTG AAC TTC AAC AGC GAG ACC GTG CCC CAG AAG
TCC TCC CTT GAA GAA CCG GAT TTT TAT AAA ACT AAA ATC AAG CTC TGC ATA CTT CTT CAT
AGC AGC CTG GAG GAG CCC GAC TTC TAC AAG ACC AAG ATC AAG CTG TGC ATC CTG CTG CAC
AGC AGC CTG GAG GAG CCC GAC TTC TAC AAG ACC AAG ATC AAG CTG TGC ATC CTG CTG CAC
GCT TTC AGA ATT CGG GCA GTG ACT ATT GAC AGA GTG ACG AGC TAT CTG AAT GCT TCC TAA
GCC TTC CGC ATC CGC GCC GTG ACC ATC GAC CGC GTG ACC AGC TAC CTG AAC GCC ACC TGA
GCC TTC CGC ATC CGG GCC GTG ACC ATC GAC CGC GTG ACC AGC TAC CTG AAC GCC ACG TGA

Additional Optimized Sequences Coding For IL-12 p35 Subunit

First line: SEQ.ID.NO.5

Second line: SEQ.ID.NO.24

Third line: SEQ.ID.NO.26

Fourth line: SEQ.ID.NO.27

Fifth line: SEQ.ID.NO.28

Sixth line: SEQ.ID.NO.29

Seventh line: SEQ.ID.NO.30

Eighth line: SEQ.ID.NO.31

```
                                                                        10 20
Met Cys Pro Ala Arg Ser Leu Leu Leu Val Ala Thr Leu Val Leu Leu Asp His Leu Ser
ATG TGY CCN GCN MGN WSN YTN YTN YTN GTN GCN ACN YTN GTN YTN YTN GAY CAY YTN WSN
--- --- --- --- --- --- --- --- --- --- --- --- --- --- --- --- --- --- --- ---
ATG TGT CCT GCT CGT TCT TTA TTA TTA GTT GCT ACT TTA GTT TTA TTA GAT CAT TTA TCT
        TGC CCC GCC CGC TCC TTG TTG TTG GTC GCC ACC TTG GTC TTG TTG GAC CAC TTG TCC
                CCA GCA CGA TCA CTT CTT CTT GTA GCA ACA CTT GTA CTT CTT CTT TCA
                CCG GCG CGG TCG CTC CTC CTC GTG GCG ACG CTC GTG CTC CTC CTC TCG
                                AGA AGT CTA CTA CTA CTA CTA CTA CTA AGT
                                AGG AGC CTG CTG CTG CTG CTG CTG CTG AGC
                                                                        30 40
Leu Ala Arg Asn Leu Pro Val Ala Thr Pro Asp Pro Gly Met Phe Pro Cys Leu His His
YTN GCN MGN AAY YTN CCN GTN GCN ACN CCN GAY CCN GGN ATG TTY CCN TGY YTN CAY CAY
--- --- --- --- --- --- --- --- --- --- --- --- --- --- --- --- --- --- --- ---
TTA GCT CGT AAT TTA CCT GTT GCT ACT CCT GAT CCT GGT ATG TTT CCT TGT TTA CAT CAT
TTG GCC CGC AAC TTG CCC GTC GCC ACC CCC GAC CCC GGC TTC CCC TGC TTG CAC CAC
CTT GCA CGA CTT CCA GTA GCA ACA CCA CCA GGA CCA CTT
CTC GCG CGG CTC CCG GTG GCG ACG CCG CCG GGG CCG CTC
CTA AGA CTA CTA
CTG AGG CTG CTG
                                                                          50 60
Ser Gln Asn Leu Leu Arg Ala Val Ser Asn Met Leu Gln Lys Ala Arg Gln Thr Leu Glu
WSN CAA AAY YTN YTN MGN GCN GTN WSN AAY ATG YTN CAA AAA GCN MGN CAA ACN YTN GAA
--- --- --- --- --- --- --- --- --- --- --- --- --- --- --- --- --- --- --- ---
TCT CAA AAT TTA TTA CGT GCT GTT TCT AAT ATG TTA CAA AAA GCT CGT CAA ACT TTA GAA
TCC CAG AAC TTG TTG CGC GCC GTC TCC AAC TTG CAG AAG GCC CGC CAG ACC TTG GAG
TCA CTT CTT CGA GCA GTA TCA CTT GCA CGA ACA CTT
TCG CTC CTC CGG GCG GTG TCG CTC GCG CGG ACG CTC
AGT CTA CTA AGA AGT CTA AGA CTA
AGC CTG CTG AGG AGC CTG AGG CTG
                                                                          70 80
Phe Tyr Pro Cys Thr Ser Glu Glu Ile Asp His Glu Asp Ile Thr Lys Asp Lys Thr Ser
TTY TAY CCN TGY ACN WSN GAA GAA ATH GAY CAY GAA GAY ATH ACN AAA GAY AAA ACN WSN
--- --- --- --- --- --- --- --- --- --- --- --- --- --- --- --- --- --- --- ---
TTT TAT CCT TGT ACT TCT GAA GAA ATT GAT CAT GAA GAT ATT ACT AAA GAT AAA ACT TCT
TTC TAC CCC TGC ACC TCC GAG GAG ATC GAC CAC GAG GAC ATC ACC AAG GAC AAG ACC TCC
                  CCA ACA TCA ATA ATA ACA ACA TCA
                  CCG ACG TCG ACG ACG TCG
                                        AGT AGT
                                        AGC AGC
                                                                          90 100
Thr Val Glu Ala Cys Leu Pro Leu Glu Leu Thr Lys Asn Giu Ser Cys Leu Asn Ser Arg
ACN GTN GAA GCN TGY YTN CCN YTN GAA YTN ACN AAA AAY GAA WSN TGY YTN AAY WSN MGN
--- --- --- --- --- --- --- --- --- --- --- --- --- --- --- --- --- --- --- ---
ACT GTT GAA GCT TGT TTA CCT TTA GAA TTA ACT AAA AAT GAA TCT TGT TTA AAT TCT CGT
ACC GTC GAG GCC TGC TTG CCC TTG GAG TTG ACC AAG AAC GAG TCC TGC TTG AAC TCC CGC
ACA GTA GCA CTT CCA CTT CTT ACA TCA CTT TCA CGA
ACG GTG GCG CTC CCG CTC CTC ACG TCG CTC TCG CGG
                                        CTA CTA CTA AGT CTA AGT AGA
                                        CTG CTG CTG AGC CTG AGC AGG
                                                                        110 120
Glu Thr Ser Phe Ile Thr Asn Gly Ser Cys Leu Ala Ser Arg Lys Thr Ser Phe Met Met
GAA ACN WSN TTY ATH ACN AAY GGN WSN TGY YTN GCN WSN MGN AAA ACN WSN TTY ATG ATG
--- --- --- --- --- --- --- --- --- --- --- --- --- --- --- --- --- --- --- ---
GAA ACT TCT TTT ATT ACT AAT GGT TCT TGT TTA GCT TCT CGT AAA ACT TCT TTT ATG ATG
GAG ACC TCC TTC ATC ACC AAC GGC TCC TGC TTG GCC TCC CGC AAG ACC TCC TTC
        ACA TCA ATA ACA GGA TCA CTT GCA TCA CGA ACA TCA
        ACG TCG ACG GGG TCG CTC GCG TCG CGG ACG TCG
                AGT AGT CTA AGT AGA AGT
                AGC AGC CTG AGC AGG AGC
                                                                        130 140
Ala Leu Cys Leu Ser Ser lie Tyr Glu Asp Leu Lys Met Tyr Gln Val Glu Phe Lys Thr
GCN YTN TGY YTN WSN WSN ATE TAY GAA GAY YTN AAA ATG TAY CAA GTN GAA TTY AAA ACN
--- --- --- --- --- --- --- --- --- --- --- --- --- --- --- --- --- --- --- ---
GCT TTA TGT TTA TCT TCT ATT TAT GAA GAT TTA AAA ATG TAT CAA GTT GAA TTT AAA ACT
GCC TTG TGC TTG TCC TCC ATC TAC GAG GAC TTG AAG TAC CAG GTC GAG TTC AAG ACC
GCA CTT CTT TCA TCA ATA CTT GTA ACA
GCG CTC CTC TCG TCG CTC GTG ACG
        CTA CTA AGT AGT CTA
        CTG CTG AGC AGC CTG
                                                                        150 160
Met Asn Ala Lys Leu Leu Met Asp Pro Lys Arg Gln Ile Phe Leu Asp Gln Asn Met Leu
ATG AAY GCN AAA YTN YTN ATG GAY CCN AAA MGN CAA ATE TTY YTN GAY CAA AAY ATG YTN
--- --- --- --- --- --- --- --- --- --- --- --- --- --- --- --- --- --- --- ---
ATG AAT GCT AAA TTA TTA ATG GAT CCT AAA CGT CAA ATT TTT TTA GAT CAA AAT ATG TTA
        AAC GCC AAG TTG TTG GAC CCC AAG CGC CAG ATC TTC TTG GAC CAG AAC TTG
                GCA CTT CTT CCA CGA ATA CTT CTT
                GCG CTC CTC CCG CGG CTC CTC
                                CTA CTA AGA CTA CTA
                                CTG CTG AGG CTG CTG
                                                                        170 180
Ala Val Ile Asp Glu Leu Met Gln Ala Leu Asn Phe Asn Ser Glu Thr Val Pro Gln Lys
GCN GTN ATE GAY GAA YTN ATG CAA GCN YTN AAY TTY AAY WSN GAA ACN GTN CCN CAA AAA
--- --- --- --- --- --- --- --- --- --- --- --- --- --- --- --- --- --- --- ---
GCT GTT ATT GAT GAA TTA ATG CAA GCT TTA AAT TTT AAT TCT GAA ACT GTT CCT CAA AAA
GCC GTC ATC GAC GAG TTG CAG GCC TTG AAC TTC AAC TCC GAG ACC GTC CCC CAG AAG
GCA GTA ATA CTT GCA CTT TCA ACA GTA CCA
GCG GTG CTC GCG CTC TCG ACG GTG CCG
                                        CTA CTA AGT
                                        CTG CTG AGC
                                                                        190 200
Ser Ser Leu Glu Glu Pro Asp Phe Tyr Lys Thr Lys Ile Lys Leu Cys Ile Leu Leu His
WSN WSN YTN GAR GAR CCN GAY TTY TAY AAR ACN AAR ATH AAR YTN TGY ATH YTN YTN CAY
--- --- --- --- --- --- --- --- --- --- --- --- --- --- --- --- --- --- --- ---
TCT TCT TTA GAA GAA CCT GAT TTT TAT AAA ACT AAA ATT AAA TTA TGT ATT TTA TTA CAT
TCC TCC TTG GAG GAG CCC GAC TTC TAC AAG ACC AAG ATC AAG TTG TGC ATC TTG TTG CAC
TCA TCA CTT CCA ACA ATA CTT ATA CTT CTT
TCG TCG CTC CCG ACG CTC CTC CTC
AGT AGT CTA CTA CTA CTA
AGC AGC CTG CTG CTG CTG
                                                                        210 220
Ala Phe Arg lie Arg Ala Val Thr Ile Asp Arg Val Thr Ser Tyr Leu Asn Ala Ser ***
GCN TTY MGN ATH MGN GCN GTN ACN ATH GAY MGN GTN ACN WSN TAY YTN AAY GCN WSN TAA
--- --- --- --- --- --- --- --- --- --- --- --- --- --- --- --- --- --- --- ---
GCT TTT CGT ATT CGT GCT GTT ACT ATT GAT CGT GTT ACT TCT TAT TTA AAT GCT TCT TAA
GCC TTC CGC ATC CGC GCC GTC ACC ATC GAC CGC GTC ACC TCC TAC TTG AAC GCC TCC TAG
GCA CGA ATA CGA GCA GTA ACA ATA CGA GTA ACA TCA CTT GCA TCA TGA
GCG CGG CGG GCG GTG ACG CGG GTG ACG TCG CTC GCG TCG
                AGA AGA AGA AGT CTA AGT
                AGG AGG AGG AGC CTG AGC
```

### Sequences Encoding Human IL-12 p40

First line: natural sequence (SEQ ID NO. 2)

Second line: all codons optimized (SEQ ID NO. 3)

Third line: all codons optimized except when same nucleic acids were too close/abundant (changes between second and third lines bolded) (SEQ ID NO. 4)

ATG TGT CAC CAG CAG TTG GTC ATC TCT TGG TTT TCC CTG GTT TTT CTG GCA TCT CCC CTC
ATG TGC CAC CAG CAG CTG GTG ATC AGC TGG TTC AGC CTG GTG TTC CTG GCC AGC CCC CTG
ATG TGC CAC CAG CAG CTG GTG ATC AGC TGG TTC TCC CTG GTG TTT CTG GCC AGC CCC CTC
GTG GCC ATA TGG GAA CTG AAG AAA GAT GTT TAT GTC GTA GAA TTG GAT TGG TAT CCG GAT
GTG GCC ATC TGG GAG CTG AAG AAG GAC GTG TAC GTG GTG GAG CTG GAC TGG TAC CCC GAC
GTG GCC ATC TGG GAG CTG AAG AAA GAC GTG TAC GTG GTC GAG CTG GAC TGG TAC CCC GAC
GCC CCT GGA GAA ATG GTG GTC CTC ACC TGT GAC ACC CCT GAA GAA GAT GGT ATC ACC TGG
GCC CCC GGC GAG ATG GTG GTG CTG ACC TGC GAC ACC CCC GAG GAG GAC GGC ATC ACC TGG
GCC CCC GGC GAG ATG GTG GTC CTG ACC TGC GAC ACC CCC GAG GAA GAC GGC ATC ACC TGG
ACC TTG GAC CAG AGC AGT GAG GTC TTA GGC TCT GGC AAA ACC CTG ACC ATC CAA GTC AAA
ACC CTG GAC CAG AGC AGC GAG GTG CTG GGC AGC GGC AAG ACC CTG ACC ATC CAG GTG AAG
ACC CTG GAC CAG AGC AGT GAG GTG CTG GGC TCC GGC AAG ACC CTG ACC ATC CAG GTG AAG
GAG TTT GGA GAT GCT GGC CAG TAC ACC TGT CAC AAA GGA GGC GAG GTT CTA AGC CAT TCG
GAG TTC GGC GAC GCC GGC CAG TAC ACC TGC CAC AAG GGC GGC GAG GTG CTG AGC CAC AGC
GAG TTC GGC GAC GCC GGC CAG TAC ACC TGC CAC AAG GGA GGC GAG GTG CTG AGC CAC TCC
CTC CTG CTG CTT CAC AAA AAG GAA GAT GGA ATT TGG TCC ACT GAT ATT TTA AAG GAC CAG
CTG CTG CTG CTG CAC AAG AAG GAG GAC GGC ATC TGG AGC ACC GAC ATC CTG AAG GAC CAG
CTC CTG CTG CTC CAC AAA AAG GAG GAC GGC ATC TGG AGC ACC GAC ATC CTG AAG GAC CAG
AAA GAA CCC AAA AAT AAG ACC TTT CTA AGA TGC GAG GCC AAG AAT TAT TCT GGA CGT TTC
AAG GAG CCC AAG AAC AAG ACC TTC CTG CGC TGC GAG GCC AAG AAC TAC AGC GGC CGC TTC
AAG GAG CCC AAG AAC AAG ACC TTC CTG CGC TGC GAG GCC AAG AAC TAC AGC GGC CGC TTC
ACC TGC TGG TGG CTG ACG ACA ATC AGT ACT GAT TTG ACA TTC AGT GTC AAA AGC AGC AGA
ACC TGC TGG TGG CTG ACC ACC ATC AGC ACC GAC CTG ACC TTC AGC GTG AAG AGC AGC AGG
ACC TGC TGG TGG CTG ACC ACG ATC AGC ACC GAC CTG ACC TTC AGT GTG AAG AGC AGC AGG
GGC TCT TCT GAC CCC CAA GGG GTG ACG TGC GGA GCT GCT ACA CTC TCT GCA GAG AGA GTC
GGC AGC AGC GAC CCC CAG GGC GTG ACC TGC GGC CCC GCC ACC CTG AGC GCC GAG CGC GTG
GGC TCC AGC GAC CCC CAG GGC GTG ACC TGC GGC GCT GCC ACC CTG AGC GCC GAG CGC GTG
AGA GGG GAC AAC AAG GAG TAT GAG TAC TCA GTG GAG TOC CAG GAG GAC AGT GCC TGC CCA
CGC GGC GAC AAC AAG GAG TAC GAG TAC AGC GTG GAG TGC CAG GAG GAC AGC GCC TGC CCC
CGC GGC GAC AAC AAG GAG TAC GAG TAC AGC GTG GAG TGC CAG GAA GAC TCC GCC TGC CCC
GCT GCT GAG GAG AGT CTG CCC ATT GAG GTC ATG GTG GAT GCC GTT CAC AAG CTC AAG TAT
GCC GCC GAG GAG AGC CTG CCC ATC GAG GTG ATG GTG GAC GCC GTC CAC AAG CTG AAG TAC
GCC GCT GAG GAG AGC CTG CCC ATC GAG GTG ATG GTG GAC GCC GTT CAC AAG CTG AAG TAC
GAA AAC TAC ACC AGC AGC TTC TTC ATC AGG GAC ATC ATC AAA CCT GAC CCA CCC AAG AAC
GAG AAC TAC ACC AGC AGC TTC TTC ATC CGC GAC ATC ATC AAG CCC GAC CCC CCC AAG AAC
GAG AAC TAC ACC AGC AGC TTC TTC ATC CGC GAC ATC ATC AAG CCT GAC CCA CCC AAG AAC
TTG CAG CTG AAG CCA TTA AAG AAT TCT CGG CAG GTG GAG GTC AGC TGG GAG TAC CCT GAC
CTG CAG CTG AAG CCC CTG AAG AAC AGC CGC CAG GTG GAG GTG AGC TGG GAG TAC CCC GAC
CTC CAG CTG AAG CCC CTC AAG AAC TCC CGC CAG GTG GAG GTG AGC TGG GAG TAC CCC GAC
ACC TGG AGT ACT CCA CAT TCC TAC TTC TCC CTG ACA TTC TGC GTT CAG GTC CAG GGC AAG
ACC TGG AGC ACC CCC CAC AGC TAC TTC AGC CTG ACC TTC TGC GTG CAG GTG CAG GGC AAG
ACC TGG AGC ACG CCC CAC TCC TAC TTC TCC CTG ACC TTC TGC GTG CAG GTC CAG GGC AAG
AGC AAG AGA GAA AAG AAA GAT AGA GTC TTC ACG GAC AAG ACC TCA GCC ACG GTC ATC TGC
AGC AAG CGC GAG AAG AAG GAC CGC GTG TTC ACC GAC AAG ACC AGC GCC ACC GTG ATC TGC
AGC AAG CGC GAG AAG AAA GAC COG GTG TTC ACC GAC AAG ACC AGC GCC ACC GTC ATC TGC
CGC AAA AAT GCC AGC ATT AGC GTG CGG GCC CAG GAC CGC TAC TAT AGC TCA TCT TGG AGC
CGC AAG AAC GCC AGC ATC AGC GTG CGC GCC CAG GAC CGC TAC TAC AGC AGC AGC TGG AGC
CGC AAG AAC GCC AGC ATC AGC GTG CGC CCC CAG GAC CGC TAC TAT AGC TCC TCT TGG AGC
GAA TGG GCA TCT GTG CCC TGC AGT TAG
GAG TGG GCC AGC GTG CCC TGC AGC TAG
GAG TGG GCC AGC GTG CCC TGC TCC TAG

### Additional Optimized Sequences Coding For IL-12 p40 Subunit

First line: SEQ.ID.NO.1

Second line: SEQ.ID.NO.25

Third line: SEQ.ID.NO.32

Fourth line: SEQ.ID.NO.33

Fifth line: SEQ.ID.NO.34

Sixth line: SEQ.ID.NO.35

Seventh line: SEQ.ID.NO.36

Eighth line: SEQ.ID.NO.37

```
                                                                          10 20
Met Cys His Gln Gln Leu Val Ile Ser Trp Phe Ser Leu Val Phe Leu Ala Ser Pro Leu
ATG TGY CAY CAA CAA YTN GTN ATH WSN TGG TTY WSN YTN GTN TTY YTN GCN WSN CCN YTN
--- --- --- --- --- --- --- --- --- --- --- --- --- --- --- --- --- --- --- ---
ATG TGT CAT CAA CAA TTA GTT ATT TCT TGG TTT TCT TTA GTT TTT TTA GCT TCT CCT TTA
        TGC CAC CAG CAG TTG GTC ATC TCC TTC TCC TTG GTC TTC TTG GCC TCC CCC TTG
                                        CTT GTA ATA TCA TCA CTT GTA CTT GCA TCA CCA CTT
                                        CTC GTG TCG TCG CTC GTG CTC GCG TCG CCG CTC
                                        CTA AGT AGT CTA CTA AGT CTA
                                        CTG AGC AGC CTG CTG AGC CTG
                                                                          30 40
Val Ala Ile Trp Glu Leu Lys Lys Asp Val Tyr Val Val Glu Leu Asp Trp Tyr Pro Asp
GTN GCN ATH TGG GAA YTN AAA AAA GAY GTN TAY GTN GTH GAA YTN GAY TGG TAY CCN GAY
--- --- --- --- --- --- --- --- --- --- --- --- --- --- --- --- --- --- --- ---
GTT GCT ATT TGG GAA TTA AAA AAA GAT GTT TAT GTT GTT GAA TTA GAT TGG TAT CCT GAT
GTC GCC ATC GAG TTG AAG AAG GAC GTC TAC GTC GTC GAG TTG GAC TAC CCC GAC
GTA GCA ATA CTT GTA GTA GTA CTT CCA
GTG GCG CTC GTG GTG GTG CTC CCG
                                        CTA CTA
                                        CTG CTG
                                                                          50 60
Ala Pro Gly Glu Met Val Val Leu Thr Cys Asp Thr Pro Glu Glu Asp Gly Ile Thr Trp
GCN CCH GGN GAA ATG GTN GTN YTN ACH TGY GAY ACN CCN GAA GAA GAY GGN ATH ACN TGG
--- --- --- --- --- --- --- --- --- --- --- --- --- --- --- --- --- --- --- ---
GCT CCT GGT GAA ATG GTT GTT TTA ACT TGT GAT ACT CCT GAA GAA GAT GGT ATT ACT TGG
GCC CCC GGC GAG GTC GTC TTG ACC TGC GAC ACC CCC GAG GAG GAC GGC ATC ACC
GCA CCA GGA GTA GTA CTT ACA ACA CCA GGA ATA ACA
GCG CCG GGG GTG GTG CTC ACG ACG CCG GGG ACG
                                                        CTA
                                                        CTG
                                                                          70 80
Thr Leu Asp Gln Ser Ser Glu Val Leu Gly Ser Gly Lys Thr Leu Thr Ile Gln Val Lys
ACN YTN GAY CAR WSN WSN GAR GTN YTN GGN WSN GGH AAR ACN YTN ACN ATH CAA GTN AAR
--- --- --- --- --- --- --- --- --- --- --- --- --- --- --- --- --- --- --- ---
ACT TTA GAT CAA TCT TCT GAA GTT TTA GGT TCT GGT AAA ACT TTA ACT ATT CAA GTT AAA
ACC TTG GAC CAG TCC TCC GAG GTC TTG GGC TCC GGC AAG ACC TTG ACC ATC CAG GTC AAG
ACA CTT TCA TCA GTA CTT GGA TCA GGA ACA CTT ACA ATA GTA
ACG CTC TCG TCG GTG CTC GGG TCG GGG ACG CTC ACG GTG
        CTA AGT AGT CTA AGT CTA
        CTG AGC AGC CTG AGC CTG
                                                                          90 100
Glu Phe Gly Asp Ala Gly Gln TYr Thr Cys His Lys Gly Gly Glu Val Leu Ser His Ser
GAR TTY GGN GAY GCN GGN CAR TAY ACN TGY CAY AAR GGN GGN GAR GTN YTN WSN CAY WSN
--- --- --- --- --- --- --- --- --- --- --- --- --- --- --- --- --- --- --- ---
GAA TTT GGT GAT GCT GGT CAA TAT ACT TGT CAT AAA GGT GGT GAA GTT TTA TCT CAT TCT
GAG TTC GGC GAC GCC GGC CAG TAC ACC TGC CAC AAG GGC GGC GAG GTC TTG TCC CAC TCC
                GGA GCA GGA ACA GGA GGA GTA CTT TCG TCG
                GGG GCG GGG ACG GGG GGG GTG CTC TCG AGT
                                                                                                                                CTA AGT AGT
                                                                                                                                CTG AGC AGC
                                                                        110 120
Leu Leu Leu Leu His Lys Lys Glu Asp Gly Ile Trp Ser Thr Asp Ile Leu Lys Asp Gln
YTN YTN YTN YTN CAY AAR AAR GAR GAY GGN ATH TGG WSN ACN GAY ATH YTN AAR GAY CAR
--- --- --- --- --- --- --- --- --- --- --- --- --- --- --- --- --- --- --- ---
TTA TTA TTA TTA CAT AAA AAA GAA GAT GGT ATT TGG TCT ACT GAT ATT TTA AAA GAT CAA
TTG TTG TTG TTG CAC AAG AAG GAG GAC GGC ATC TCC ACC GAC ATC TTG AAG GAC CAG
CTT CTT CTT CTT GGA ATA TCA ACA ATA CTT
CTC CTC CTC CTC GGG TCG ACG CTC
CTA CTA CTA CTA AGT CTA
CTG CTG CTG CTG AGC CTG
                                                                        130 140
Lys Glu Pro Lys Asn Lys Thr Phe Leu Arg Cys Glu Ala Lys Asn Tyr Ser Gly Arg Phe
AAR GAR CCN AAR AAY AAR ACN TTY YTN MGN TGY GAR GCN AAR AAY TAY WSN GGN MGN TTY
--- --- --- --- --- --- --- --- --- --- --- --- --- --- --- --- --- --- --- ---
AAA GAA CCT AAA AAT AAA ACT TTT TTA CGT TGT GAA GCT AAA AAT TAT TCT GGT CGT TTT
AAG GAG CCC AAG AAC AAG ACC TTC TTG CGC TGC GAG GCC AAG AAC TAC TCC GGC CGC TTC
                CCA ACA CTT CGA GCA TCA GGA CGA
                CCG ACG CTC CGG GCG TCG GGG CGG
                                                                CTA AGA AGT AGA
                                                                CTG AGG AGC AGG
                                                                        150 160
Thr Cys Trp Trp Leu Thr Thr Ile Ser Thr Asp Leu Thr Phe Ser Val Lys Ser Ser Arg
ACN TGY TGG TGG YTN ACN ACN ATH WSW ACN GAY YTN ACN TTY WSN GTN AAR WSN WSN MGN
--- --- --- --- --- --- --- --- --- --- --- --- --- --- --- --- --- --- --- ---
ACT TGT TGG TGG TTA ACT ACT ATT TCT ACT GAT TTA ACT TTT TCT GTT AAA TCT TCT CGT
ACC TGC TTG ACC ACC ATC TCC ACC GAC TTG ACC TTC TCC GTC AAG TCC TCC CGC
ACA CTT ACA ACA ATA TCA ACA CTT ACA TCA GTA TCA TCA CGA
ACG CTC ACG ACG TCG ACG CTC ACG TCG GTG TCG TCG CGG
                                CTA AGT CTA AGT AGT AGT AGA
                                CTG AGC CTG AGC AGC AGC AGG
                                                                        170 180
Gly Ser Ser Asp Pro Gln Gly Val Thr Cys Gly Ala Ala Thr Leu Ser Ala Glu Arg Val
GGN WSN WSN GAY CCN CAR GGN GTN ACN TGY GGN GCN GCN ACN YTN WSW GCN GAR MGN GTN
--- --- --- --- --- --- --- --- --- --- --- --- --- --- --- --- --- --- --- ---
GGT TCT TCT GAT CCT CAA GGT GTT ACT TGT GGT GCT GCT ACT TTA TCT GCT GAA CGT GTT
GGC TCC TCC GAC CCC CAG GGC GTC ACC TGC GGC GCC GCC ACC TTG TCC GCC GAG CGC GTC
GGA TCA TCA CCA GGA GTA ACA GGA GCA GCA ACA CTT TCA GCA CGA GTA
GGG TCG TCG CCG GGG GTG ACG GGG GCG GCG ACG CTC TCG GCG CGG GTG
        AGT AGT CTA AGT AGA
        AGC AGC CTG AGC AGG
                                                                        190 200
Arg Gly Asp Asn Lys Glu Tyr Glu Tyr Ser Val Glu Cys Gln Glu Asp Ser Ala Cys Pro
MGN GGN GAY AAY AAA GAA TAY GAA TAY WSN GTN GAA TGY CAA GAA GAY WSN GCN TGY CCN
--- --- --- --- --- --- --- --- --- --- --- --- --- --- --- --- --- --- --- ---
CGT GGT GAT AAT AAA GAA TAT GAA TAT TCT GTT GAA TGT CAA GAA GAT TCT GCT TGT CCT
CGC GGC GAC AAC AAG GAG TAC GAG TAC TCC GTC GAG TGC CAG GAG GAC TCC GCC TGC CCC
CGA GGA TCA GTA TCA GCA CCA
CGG GGG TCG GTG TCG GCG CCG
AGA AGT AGT
AGG AGC AGC
                                                                        210 220
Ala Ala Glu Glu Ser Leu Pro Ile Glu Val Met Val Asp Ala Val His Lys Leu Lys Tyr
GCN GCN GAA GAA WSN YTN CCN ATH GAA GTN ATG GTN GAY GCN GTN CAY AAA YTN AAA TAY
--- --- --- --- --- --- --- --- --- --- --- --- --- --- --- --- --- --- --- ---
GCT GCT GAA GAA TCT TTA CCT ATT GAA GTT ATG GTT GAT GCT GTT CAT AAA TTA AAA TAT
GCC GCC GAG GAG TCC TTG CCC ATC GAG GTC GTC GAC GCC GTC CAC AAG TTG AAG TAC
GCA GCA TCA CTT CCA ATA GTA GTA GCA GTA CTT
GCG GCG TCG CTC CCG GTG GTG GCG GTG CTC
                                AGT CTA CTA
                                AGC CTG CTG
                                                                        230 240
Glu Asn Tyr Thr Ser Ser Phe Phe Ile Arg Asp Ile Ile Lys Pro Asp Pro Pro Lys Asn
GAR AAY TAY ACN WSN WSN TTY TTY ATH MGN GAY ATH ATH AAA CCN GAY CCN CCN AAR AAY
--- --- --- --- --- --- --- --- --- --- --- --- --- --- --- --- --- --- --- ---
GAA AAT TAT ACT TCT TCT TTT TTT ATT CGT GAT ATT ATT AAA CCT GAT CCT CCT AAA AAT
GAG AAC TAC ACC TCC TCC TTC TTC ATC CGC GAC ATC ATC AAG CCC GAC CCC CCC AAG AAC
                        ACA TCA TCA ATA CGA ATA ATA CCA CCA CCA
                        ACG TCG TCG CGG CCG CCG CCG
                                AGT AGT AGA
                                AGC AGC AGG
                                                                        250 260
Leu Gln Leu Lys Pro Leu Lys Asn Ser Arg Gln Val Glu Val Ser Trp Glu Tyr Pro Asp
YTN CAA YTN AAA CCN YTN AAA AAY WSN MGN CAA GTN GAA GTN WSN TGG GAA TAY CCN GAY
--- --- --- --- --- --- --- --- --- --- --- --- --- --- --- --- --- --- --- ---
TTA CAA TTA AAA CCT TTA AAA AAT TCT CGT CAA GTT GAA GTT TCT TGG GAA TAT CCT GAT
TTG CAG TTG AAG CCC TTG AAG AAC TCC CCC CAG GTC GAG GTC TCC GAG TAC CCC GAC
CTT CTT CCA CTT TCA CGA GTA GTA TCA CCA
CTC CTC CCG CTC TCG CGG GTG GTG TCG CCG
CTA CTA CTA AGT AGA AGT
CTG CTG CTG AGC AGG AGC
                                                                        270 280
Thr Trp Ser Thr Pro His Ser Tyr Phe Ser Leu Thr Phe Cys Val Gln Val Gln Gly Lys
ACN TGG WSN ACN CCN CAY WSN TAY TTY WSN YTN ACN TTY TGY GTN CAA GTN CAA GGN AAA
--- --- --- --- --- --- --- --- --- --- --- --- --- --- --- --- --- --- --- ---
ACT TGG TCT ACT CCT CAT TCT TAT TTT TCT TTA ACT TTT TGT GTT CAA GTT CAA GCT AAA
ACC TCC ACC CCC CAC TCC TAC TTC TCC TTG ACC TTC TGC GTC CAG GTC CAG GGC AAG
ACA TCA ACA CCA TCA TCA CTT ACA GTA GTA GGA
ACG TCG ACG CCG TCG TCG CTC ACG GTG GTG GGG
                AGT AGT AGT CTA
                AGC AGC AGC CTG
                                                                        290 300
Ser Lys Arg Glu Lys Lys Asp Arg Val Phe Thr Asp Lys Thr Ser Ala Thr Val Ile Cys
WSN AAR MGN GAA AAA AAA GAY MGN GTN TTY ACN GAY AAA ACN WSN GCN ACN GTN ATH TGY
--- --- --- --- --- --- --- --- --- --- --- --- --- --- --- --- --- --- --- ---
TCT AAA CGT GAA AAA AAA GAT CGT GTT TTT ACT GAT AAA ACT TCT GCT ACT GTT ATT TGT
TCC AAG CGC GAG AAG AAG GAC CGC GTC TTC ACC GAC AAG ACC TCC GCC ACC GTC ATC TGC
TCA CGA CGA GTA ACA ACA TCA GCA ACA GTA ATA
TCG CGG CGG GTG ACG ACG TCG GCG ACG GTG
AGT AGA AGA AGT
AGC AGG AGG AGC
                                                                        310 320
Arg Lys Asn Ala Ser Ile Ser Val Arg Ala Gln Asp Arg Tyr Tyr Ser Ser Ser Trp Ser
MGN AAR AAY GCN WSN ATE WSN GTN MGN GCN CAA GAY MGN TAY TAY WSN WSN WSN TGG WSN
--- --- --- --- --- --- --- --- --- --- --- --- --- --- --- --- --- --- --- ---
CGT AAA AAT GCT TCT ATT TCT GTT CGT GCT CAA GAT CGT TAT TAT TCT TCT TCT TGG TCT
CGC AAG AAC GCC TCC ATC TCC GTC CGC GCC CAG GAC CGC TAC TAC TCC TCC TCC TCC
CGA GCA TCA ATA TCA GTA CGA GCA CGA TCA TCA TCA TCA
CGG GCG TCG TCG GTG CGG GCG CGG TCG TCG TCG TCG
AGA AGT AGT AGA AGA AGT AGT AGT AGT
AGG AGC AGC AGG AGG AGC AGC AGC AGC
Glu Trp Ala Ser Val Pro Cys Ser ***
GAR TGG GCN WSN GTN CCN TGY WSN TRR
--- --- --- --- --- --- --- --- ---
GAA TGG GCT TCT GTT CCT TGT TCT TAA
GAG GCC TCC GTC CCC TGC TCC TAG
                GCA TCA GTA CCA TCA TGA
                GCG TCG GTG CCG TCG
                        AGT AGT
                        AGC AGC
```

### Wild Type Sequence Coding for Interferon Alpha

| | |
|---|---|
| 9 18 27 36 45 54 | |
| 5′ ATG GCC TTG ACC TTT GCT TTA CTG GTG GCC CTC CTG GTG CTC AGC TGC AAG TCA | |
| --- --- --- --- --- --- --- --- --- --- --- --- --- --- --- --- --- --- | |
| M A L T F A L L V A L L V L S C K S | |
| 63 72 81 90 99 | 108 |
| AGC TGC TCT GTG GGC TGT GAT CTG CCT CAA ACC CAC AGC CTG GGT AGC AGG AGG | |
| --- --- --- --- --- --- --- --- --- --- --- --- --- --- --- --- --- --- | |
| S C S V G C D L P Q T H S L G S R R | |
| 117 126 135 144 153 162 | |
| ACC TTG ATG CTC CTG GCA CAG ATG AGG AGA ATC TCT CTT TTC TCC TGC TTG AAG | |
| --- --- --- --- --- --- --- --- --- --- --- --- --- --- --- --- --- --- | |
| T L M L L A Q M R R I S L F S C L K | |
| 171 180 189 198 207 216 | |
| GAC AGA CAT GAC TTT GGA TTT CCC CAG GAG GAG TTT GGC AAC CAG TTC CAA AAG | |
| --- --- --- --- --- --- --- --- --- --- --- --- --- --- --- --- --- --- | |
| D R H D F G F P Q E E F G N Q F Q K | |
| 225 234 243 252 261 270 | |
| GCT GAA ACC ATC CCT GTC CTC CAT GAG ATG ATC CAG CAG ATC TTC AAT CTC TTC | |
| --- --- --- --- --- --- --- --- --- --- --- --- --- --- --- --- --- --- | |
| A E T I P V L H E M I Q Q I F N L F | |
| 279 288 297 306 315 324 | |
| AGC ACA AAG GAC TCA TCT GCT GCT TGG GAT GAG ACC CTC CTA GAC AAA TTC TAC | |
| --- --- --- --- --- --- --- --- --- --- --- --- --- --- --- --- --- --- | |
| S T K D S S A A W D E T L L D K F Y | |
| 333 342 351 360 369 378 | |
| ACT GAA CTC TAC CAG CAG CTG AAT GAC CTG GAA GCC TGT GTG ATA CAG GGG GTG | |
| --- --- --- --- --- --- --- --- --- --- --- --- --- --- --- --- --- --- | |
| T E L Y Q Q L N D L E A C V I Q G V | |
| 387 396 405 414 423 432 | |
| GGG GTG ACA GAG ACT CCC CTG ATG AAG GAG GAC TCC ATT CTG GCT GTG AGG AAA | |
| --- --- --- --- --- --- --- --- --- --- --- --- --- --- --- --- --- --- | |
| G V T E T P L M K E D S I L A V A K | |
| 441 450 459 468 477 486 | |
| TAC TTC CAA AGA ATC ACT CTC TAT CTG AAA GAG AAG AAA TAC AGC CCT TGT GCC | |
| --- --- --- --- --- --- --- --- --- --- --- --- --- --- --- --- --- --- | |
| Y F Q R I T L Y L K E K K Y S P C A | |
| 495 504 513 522 531 540 | |
| TGG GAG GTT GTC AGA GCA GAA ATC ATG AGA TCT TTT TCT TTG TCA ACA AAC TTG | |
| --- --- --- --- --- --- --- --- --- --- --- --- --- --- --- --- --- --- | |
| W E V V R A E I M R S F S L S T N L | |
| 549 | 558 567 |
| CAA GAA AGT TTA AGA AGT AAG GAA TGA 3′ | |
| --- --- --- --- --- --- --- --- --- | |
| Q E S L R S K E * | |

### Interferon Alpha Coding Sequence with all Codons Optimized (SEQ ID NO:11)

ATG GCC CTG ACC TTC GCC CTG CTG GTG GCC CTG CTG GTG CTG AGC TGC AAG AGC AGC TGC
TCC GTG GOG TGC GAC CTG CCC CAG ACC CAC AGC CTG GGG AGC CGG CGG ACC CTG ATG CTG
CTG GCC CAG ATG CGG CGG ATC AGC CTG TTC AGC TGC CTG AAG GAC CGG CAC GAC TTC GGG
TTC CCC CAG GAG GAG TTC GGG AAC CAG TTC CAG AAG GCC GAG ACC ATC CCC GTG CTG CAC
GAG ATG ATC CAG CAG ATC TTC AAC CTG TTC AGC ACC AAG GAC AGC AGC GCC GCC TGG GAC
GAG ACC CTG CTG GAC AAG TTC TAC ACC GAG CTG TAC CAG CAG CTG AAC GAC CTG GAG GCC
TGC GTG ATC CAG GGG GTG GGG GTG ACC GAG ACC CCC CTG ATG AAG GAG GAC AGC ATC CTG
GCC GTG CGG AAG TAC TTC CAG CGG ATC ACC CTG TAC CTG AAG GAG AAG AAG TAC TCC CCC
TGC GCC TGG GAG GTG GTG CGG GCC GAG ATC ATG CGG AGC TTC AGC CTG AGC ACC AAC CTG
CAG GAG AGC CTG CGG AGC AAG GAG TGA

### Additional/Semi Optimized Sequence Coding for Interferon Alpha

First line: SEQ.ID.NO.9

Second line: SEQ.ID.NO.12

Third line: SEQ.ID.NO.38

Fourth line: SEQ.ID.NO.39

Fifth line: SEQ.ID.NO.40

Sixth line: SEQ.ID.NO.41

Seventh line: SEQ.ID.NO.42

Eighth line: SEQ.ID.NO.43

```
                                                                          10 20
MET ALA LEU THR PHE ALA LEU LEU VAL ALA LEU LEU VAL LEU SER CYS LYS SER SER CYS
ATG GCN YTN ACN TTY GCN YTN YTN GTN GCN YTN YTN GTN YTN WSN TGY AAR WSN WSN TGY
--- --- --- --- --- --- --- --- --- --- --- --- --- --- --- --- --- --- --- ---
ATC GCT TTA ACT TTT GCT TTA TTA GTT GCT TTA TTA GTT TTA TCT TGT AAA TCT TCT TGT
        GCC TTG ACC TTC GCC TTG TTG GTC GCC TTG TTG GTC TTG TCC TGC AAG TCC TCC TGC
        GCA CTT ACA GCA CTT CTT GTA GCA CTT CTT GTA CTT TCA TCA TCA
        GCG CTC ACG GCG CTC CTC GTG GCG CTC CTC GTG CTC TCG AGT AGT
                CTA CTA CTA CTA CTA CTA AGT AGT AGT
                CTG CTG CTG CTG CTG CTG AGC AGC AGC
                                                                          30 40
SER VAL GLY CYS ASP LEU PRO GLN THR HIS SER LEU GLY SER ARG ARG THR LEU MET LEU
WSN GTN GGN TGY GAY YTN CCN CAR ACN CAY WSN YTN GGN WSN MGN MGN ACN YTN ATG YTN
--- --- --- --- --- --- --- --- --- --- --- --- --- --- --- --- --- --- --- ---
TCT GTT GGT TGT GAT TTA CCT CAA ACT CAT TCT TTA GGT TCT CGT CGT ACT TTA ATG TTA
TCC GTC GGC TGC GAC TTG CCC CAG ACC CAC TCC TTG GGC TCC CGC CGC ACC TTG TTG
TCA GTA GGA CTT CCA ACA TCA CTT GGA TCA CGA CGA ACA CTT CTT
TCG GTG GGG CTC CCG ACG TCG CTC GGG TCG CGG CGG ACG CTC CTC
AGT CTA AGT CTA AGT AGA AGA CTA CTA
AGC CTG AGC CTG AGC AGG AGG CTG CTG
                                                                          50 60
LEU ALA GLN MET ARG ARG ILE SER LEU PHE SER CYS LEU LYS ASP ARG HIS ASP PHE GLY
YTN GCN CAR ATG MGN MGN ATH WSN YTN TTY WSN TGY YTN AAR GAY MGN CAY GAY TTY GGN
--- --- --- --- --- --- --- --- --- --- --- --- --- --- --- --- --- --- --- ---
TTA GCT CAA ATG CGT CGT ATT TCT TTA TTT TCT TGT TTA AAA GAT CGT CAT GAT TTT GGT
TTG GCC CAG CGC CGC ATC TCC TTG TTC TCC TGC TTG AAG GAC CGC CAC GAC TTC GGC
CTT GCA CGA CGA ATA TCA CTT TCA CTT CGA GGA
CTC GCG CGG CGG TCG CTC TCG CTC CGG GGG
CTA AGA AGA AGT CTA AGT CTA AGA
CTG AGG AGG AGC CTG AGC CTG AGG
                                                                          70 80
PHE PRO GLN GLU GLU PHE GLY ASN GLN PHE GLN LYS ALA GLU THR ILE PRO VAL LEU HIS
TTY CCN CAR GAR GAR TTY GGN AAY CAR TTY CAR AAR GCN GAR ACN ATH CCN GTN YTN CAY
--- --- --- --- --- --- --- --- --- --- --- --- --- --- --- --- --- --- --- ---
TTT CCT CAA GAA GAA TTT GGT AAT CAA TTT CAA AAA GCT GAA ACT ATT CCT GTT TTA CAT
TTC CCC CAG GAG GAG TTC GGC AAC CAG TTC CAG AAG GCC GAG ACC ATC CCC GTC TTG CAC
        CCA GGA GCA ACA ATA CCA GTA CTT
        CCG GGG GCG ACG CCG GTG CTC
                                                                                                                                                CTA
                                                                                                                                                CTG
                                                                          90 100
GLU MET ILE GLN GLN ILE PHE ASN LEU PHE SER THR LYS ASP SER SER ALA ALA TRP ASP
GAR ATG ATH CAR CAR ATH TTY AAY YTN TTY WSN ACN AAR GAY WSN WSN GCN GCN TGG GAY
--- --- --- --- --- --- --- --- --- --- --- --- --- --- --- --- --- --- --- ---
GAA ATG ATT CAA CAA ATT TTT AAT TTA TTT TCT ACT AAA GAT TCT TCT GCT GCT TGG GAT
GAG ATC CAG CAG ATC TTC AAC TTG TTC TCC ACC AAG GAC TCC TCC GCC GCC GAC
                ATA ATA CTT TCA ACA TCA TCA GCA GCA
                                                                CTC TCG ACG TCG TCG GCG GCG
                                                                CTA AGT AGT AGT
                                                                CTG AGC AGC AGC
                                                                        110 120
GLU THR LEU LEU ASP LYS PHE TYR THR GLU LEU TYR GLN GLN LEU ASN ASP LEU GLU ALA
GAR ACN YTN YTN GAY AAR TTY TAY ACN GAR YTN TAY CAR CAR YTN AAY GAY YTN GAR GCN
--- --- --- --- --- --- --- --- --- --- --- --- --- --- --- --- --- --- --- ---
GAA ACT TTA TTA GAT AAA TTT TAT ACT GAA TTA TAT CAA CAA TTA AAT GAT TTA GAA GCT
GAG ACC TTG TTG GAC AAG TTC TAC ACC GAG TTG TAC CAG CAG TTG AAC GAC TTG GAG GCC
        ACA CTT CTT ACA CTT CTT CTT GCA
        ACG CTC CTC ACG CTC CTC CTC GCG
                CTA CTA CTA CTA CTA CTG
                CTG CTG CTG CTG CTG
                                                                        130 140
CYS VAL ILE GLN GLY VAL GLY VAL THR GLU THR PRO LEU MET LYS GLU ASP SER ILE LEU
TGY GTN ATH CAR GGN GTN GGN GTN ACN GAR ACN CCN YTN ATG AAR GAR GAY WSN ATH YTN
--- --- --- --- --- --- --- --- --- --- --- --- --- --- --- --- --- --- --- ---
TGT GTT ATT CAA GGT GTT GGT GTT ACT GAA ACT CCT TTA ATG AAA GAA GAT TCT ATT TTA
TGC GTC ATC CAG GGC GTC GGC GTC ACC GAG ACC CCC TTG AAG GAG GAC TCC ATC TTG
        GTA ATA GGA GTA GGA GTA ACA ACA CCA CTT TCA ATA CTT
        GTG GGG GTG GGG GTG ACG ACG CCG CTC TCG CTC
                                                                                                CTA AGT CTA
                                                                                                CTG AGC CTG
                                                                        150 160
ALA VAL ARG LYS TYR PHE GLN ARG ILE THR LUE TYR LEU LYS GLU LYS LYS TYR SER PRO
GCN GTN MGN AAR TAY TTY CAR MGN ATH ACN YTN TAY YTN AAR GAR AAR AAR TAY WSN CCN
--- --- --- --- --- --- --- --- --- --- --- --- --- --- --- --- --- --- --- ---
GCT GTT CGT AAA TAT TTT CAA CGT ATT ACT TTA TAT TTA AAA GAA AAA AAA TAT TCT CCT
GCC GTC CGC AAG TAC TTC CAG CGC ATC ACC TTG TAC TTG AAG GAG AAG AAG TAC TCC CCC
GCA GTA CGA CGA ATA ACA CTT CTT TCA CCA
GCG GTG CGG CGG ACG CTC CTC TCG CCG
                AGA AGA CTA CTA AGT
                AGG AGG CTG CTG AGC
                                                                        170 180
CYS ALA TRP GLU VAL VAL ARG ALA GLU ILE MET ARG SER PHE SER LEU SER THR ASN LEU
TGY GCN TGG GAR GTN GTN MGN GCN GAR ATH ATG MGN WSN TTY WSN YTN WSN ACN AAY YTN
--- --- --- --- --- --- --- --- --- --- --- --- --- --- --- --- --- --- --- ---
TGT GCT TGG GAA GTT GTT CGT GCT GAA ATT ATG CGT TCT TTT TCT TTA TCT ACT ATT TTA
TGC GCC GAG GTC GTC CGC GCC GAG ATC CGC TCC TTC TCC TTG TCC ACC AAC TTG
        GCA GTA GTA CGA GCA ATA CGA TCA TCA CTT TCA ACA CTT
        GCG GTG GTG CGG GCG CGG TCG TCG CTC TCG ACG CTC
                                                AGA AGA AGT AGT CTA AGT CTA
                                                AGG AGG AGC AGC CTG AGC CTG
GLN GLU SER LEU ARG SER LYS GLU ***
CAR GAR WSN YTN MGN WSN AAR GAR TRR
--- --- --- --- --- --- --- --- ---
CAA GAA TCT TTA CGT TCT AAA GAA TAA
CAG GAG TCC TTG CGC TCC AAG GAG TAG
                TCA CTT CGA TCA TGA
                TCG CTC CGG TCG
                AGT CTA AGA AGT
                AGC CTG AGG AGC
```

Delivery and expression of nucleic acids in many formulations is limited due to degradation of the nucleic acids by components of organisms, such as nucleases. Thus, protection of the nucleic acids when delivered in vivo can greatly enhance the resulting expression, thereby enhancing a desired pharmacological or therapeutic effect. It was found that certain types of compounds which interact with a nucleic acid (e.g., DNA) in solution but do not condense the nucleic acid provide in vivo protection to the nucleic acid, and correspondingly enhance the expression of an encoded gene product.

We have described the use of delivery systems designed to interact with plasmids and protect plasmids from rapid extracellular nuclease degradation [Mumper, R. J., et al., 1996, Pharm. Res. 13:701-709; Mumper, R. J., et al., 1997. Submitted to Gene Therapy]. A characteristic of the PINC systems is that they are non-condensing systems that allow the plasmid to maintain flexibility and diffuse freely throughout the muscle while being protected from nuclease degradation. While the PINC systems are primarily discussed below, it will be understood that cationic lipid based systems and systems utilizing both PINCS and cationic lipids are also within the scope of the present invention.

A common structural component of the PINC systems is that they are amphiphilic molecules, having both a hydrophilic and a hydrophobic portion. The hydrophilic portion of the PINC is meant to interact with plasmids by hydrogen bonding (via hydrogen bond acceptor or donor groups), Van der Waals interactions, or/and by ionic interactions. For example, PVP and N-methyl-2-pyrrolidone (NM2P) are hydrogen bond acceptors while PVA and PG are hydrogen bond donors.

All four molecules have been reported to form complexes with various (poly)anionic molecules [Buhler V., BASF Aktiengescellschaft Feinchemie, Ludwigshafen, pp 39-42; Galaev Y, et al., J. Chrom. A. 684:45-54 (1994); Tarantino R, et al. J. Pharm. Sci. 83:1213-1216 (1994); Zia, H., et al., Pharm. Res. 8:502-504 (1991);]. The hydrophobic portion of the PINC systems is designed to result in a coating on the plasmid rendering its surface more hydrophobic. Kabanov et al. have described previously the use of cationic polyvinyl derivatives for plasmid condensation designed to increase plasmid hydrophobicity, protect plasmid from nuclease degradation, and increase its affinity for biological membranes [Kabanov, A. V., and Kabanov, V. A., 1995, Bioconj. Chem. 6:7-20; Kabanov, A. V., et al., 1991, Biopolymers 31:1437-1443; Yaroslavov, A. A., et al., 1996, FEBS Letters 384:177-180].

Substantial protective effect is observed; up to at least a one log enhancement of gene expression in rat muscle over plasmid formulated in saline has been demonstrated with these exemplary PINC systems. We have also found that the expression of reporter genes in muscle using plasmids complexed with the PINC systems was more reproducible than when the plasmid was formulated in saline. For example, the coefficient of variation for reporter gene expression in muscle using plasmid formulated in saline was 96±35% (n=20 studies; 8-12 muscles/study) whereas with coefficient of variation with plasmids complexed with PINC systems was 40±19% (n=30 studies; 8-12 muscles/study). The high coefficient of variation for reporter gene expression with plasmid formulated in saline has been described previously 1-5 [Davis, H. L., et al., 1993, Hum. Gene Ther. 4:151-9]. In addition, in contrast with the results for DNA:saline, there was no significant difference in gene expression in muscle when plasmid with different topologies were complexed with polyvinyl pyrrolidone (PVP). This suggests that PVP is able to protect all forms of the plasmid from rapid nuclease degradation.

1. Summary of Interactions Between a PINC Polymer (PVP) and Plasmid

We have demonstrated using molecular modeling that an exemplary PINC polymer, PVP, forms hydrogen bonds with the base pairs of a plasmid within its major groove and results in a hydrophobic surface on the plasmid due to the vinyl backbone of PVP. These interactions are supported by the modulation of plasmid zeta potential by PVP as well as by the inhibition of ethidium bromide intercalation into complexed plasmid. We have correlated apparent binding between PVP and plasmid to pH and salt concentration and have demonstrated the effect of these parameters on β-gal expression after intramuscular injection of plasmid/PVP complexes [Mumper, R. J., et al., 1997. Submitted to Gene Therapy]. A summary of the physico-chemical properties of plasmid/PVP complexes is listed in Table I below.

TABLE I
Summary of the Physico-Chemical Properties
of Plasmid/PVP Complexes

| Method | Result |
|---|---|
| Molecular modeling | Hydrogen bonding and hydrophobic |
| | plasmid surface observed |
| Fourier-transformed | Hydrogen bonding demonstrated |
| Infra-red | |
| DNase I challenge | Decreased rate of plasmid degradation |
| | in the presence of PVP |
| Microtitration | Positive heats of reaction indicative |
| Calorimetry | of an endothermic process |
| Potentiometric titration | One unit pH drop when plasmid and PVP |
| | are complexed |
| Dynamic Dialysis | Rate of diffusion of PVP reduced in the |
| | presence of plasmid |
| Zeta potential modulation | Surface charge of plasmid decreased by |
| | PVP |
| Ethidium bromide | Ethidium bromide intercalation reduced |
| Intercalation | by plasmid/PVP complexation |
| Osmotic pressure | Hyper-osmotic formulation (i.e., 340 |
| | mOsm/kg H₂O) |
| Luminescence Spectroscopy | Plasmid/PVP binding decreased in salt |
| | and/or at pH 7 |

2. Histology of Expression in Muscle

Immunohistochemistry for β-gal using a slide scanning technology has revealed the uniform distribution of β-gal expression sites across the whole cross-sections of rat tibialis muscles. Very localized areas were stained positive for β-gal when CMV-β-gal plasmid was formulated in saline. β-gal positive cells were observed exclusively around the needle tract when plasmid was injected in saline. This is in agreement with previously published results [Wolff, J. A., et al., 1990, Science 247:1465-68; Davis, H. L., et al., 1993, Hum. Gene Ther. 4:151-9; Davis, H. L., et al., 1993, Hum. Gene Ther. 4:733-40].

In comparison, immunoreactivity for β-gal was observed in a wide area of muscle tissue after intramuscular injection of CMV-p-gal plasmid/PVP complex (1:17 w/w) in 150 mM NaCl. It appeared that the majority of positive muscle fibers were located at the edge of muscle bundles. Thus, staining for β-gal in rat muscle demonstrated that, using a plasmid/PVP complex, the number of muscle fibers stained positive for β-gal was approximately 8-fold greater than found using a saline formulation. Positively stained muscle fibers were also observed over a much larger area in the muscle tissue using the plasmid/PVP complex providing evidence that the injected plasmid was widely dispersed after intramuscular injection.

We conclude that the enhanced plasmid distribution and expression in rat skeletal muscle was a result of both protection from extracellular nuclease degradation due to complexation and hyper-osmotic effects of the plasmid/PVP complex. However, Dowty and Wolff et al. have demonstrated that osmolarity, up to twice physiologic osmolarity, did not significantly effect gene expression in muscle [Dowty, M. E., and Wolff, J. A. In: J. A. Wolff (Ed.), 1994, Gene Therapeutics: Methods and Applications of Direct Gene Transfer. Birkhauser, Boston, pp. 82-98]. This suggests that the enhanced expression of plasmid due to PVP complexation is most likely due to nuclease protection and less to osmotic effects. Further, the surface modification of plasmids by PVP (e.g., increased hydrophobicity and decreased negative surface charge) may also facilitate the uptake of plasmids by muscle cells.

3. Structure-Activity Relationship of PINC Polymers

We have found a linear relationship between the structure of a series of co-polymers of vinyl pyrrolidone and vinyl acetate and the levels of gene expression in rat muscle. We have found that the substitution of some vinyl pyrrolidone monomers with vinyl acetate monomers in PVP resulted in a co-polymer with reduced ability to form hydrogen bonds with plasmids. The reduced interaction subsequently led to decreased levels of gene expression in rat muscle after intramuscular injection. The expression of β-gal decreased linearly (R=0.97) as the extent of vinyl pyrrolidone monomer (VPM) content in the co-polymers decreased.

These data demonstrate that pH and viscosity are not the most important parameters effecting delivery of plasmid to muscle cells since these values were equivalent for all complexes. These data suggest that enhanced binding of the PINC polymers to plasmid results in increased protection and bioavailability of plasmid in muscle.

4. Additional PINC Systems

The structure-activity relationship described above can be used to design novel co-polymers that will also have enhanced interaction with plasmids. It is expected that there is “an interactive window of opportunity” whereby enhanced binding affinity of the PINC systems will result in a further enhancement of gene expression after their intramuscular injection due to more extensive protection of plasmids from nuclease degradation. It is expected that there will be an optimal interaction beyond which either condensation of plasmids will occur or “triplex” type formation, either of which can result in decreased bioavailability in muscle and consequently reduced gene expression.

As indicated above, the PINC compounds are generally amphiphilic compounds having both a hydrophobic portion and a hydrophilic portion. In many cases the hydrophilic portion is provided by a polar group. It is recognized in the art that such polar groups can be provided by groups such as, but not limited to, pyrrolidone, alcohol, acetate, amine or heterocyclic groups such as those shown on pp. 2-73 and 2-74 of CRC Handbook of Chemistry and Physics (72nd Edition), David R. Lide, editor, including pyrroles, pyrazoles, imidazoles, triazoles, dithiols, oxazoles, (iso)thiazoles, oxadiazoles, oxatriazoles, diaoxazoles, oxathioles, pyrones, dioxins, pyridines, pyridazines, pyrimidines, pyrazines, piperazines, (iso)oxazines, indoles, indazoles, carpazoles, and purines and derivatives of these groups, hereby incorporated by reference.

Compounds also contain hydrophobic groups which, in the case of a polymer, are typically contained in the backbone of the molecule, but which may also be part of a non-polymeric molecule. Examples of such hydrophobic backbone groups include, but are not limited to, vinyls, ethyls, acrylates, acrylamides, esters, celluloses, amides, hydrides, ethers, carbonates, phosphazenes, sulfones, propylenes, and derivatives of these groups. The polarity characteristics of various groups are quite well known to those skilled in the art as illustrated, for example, by discussions of polarity in any introductory organic chemistry textbook.

The ability of such molecules to interact with nucleic acids is also understood by those skilled in the art, and can be predicted by the use of computer programs which model such intermolecular interactions. Alternatively or in addition to such modeling, effective compounds can readily be identified using one or more of such tests as 1) determination of inhibition of the rate of nuclease digestion, 2) alteration of the zeta potential of the DNA, which indicates coating of DNA, 3) or inhibition of the ability of intercalating agents, such as ethidium bromide to intercalate with DNA.

5. Targeting Ligands

In addition to the nucleic acid/PINC complexes described above for delivery and expression of nucleic acid sequences, in particular embodiments it is also useful to provide a targeting ligand in order to preferentially obtain expression in particular tissues, cells, or cellular regions or compartments.

Such a targeted PINC complex includes a PINC system (monomeric or polymeric PINC compound) complexed to plasmid (or other nucleic acid molecule). The PINC system is covalently or non-covalently attached to (bound to) a targeting ligand (TL) which binds to receptors having an affinity for the ligand. Such receptors may be on the surface or within compartments of a cell. Such targeting provides enhanced uptake or intracellular trafficking of the nucleic acid.

The targeting ligand may include, but is not limited to, galactosyl residues, fucosal residues, mannosyl residues, carnitine derivatives, monoclonal antibodies, polyclonal antibodies, peptide ligands, and DNA-binding proteins. Examples of cells which may usefully be targeted include, but are not limited to, antigen-presenting cells, hepatocytes, myocytes, epithelial cells, endothelial cells, and cancer cells.

Formation of such a targeted complex is illustrated by the following example of covalently attached targeting ligand (TL) to PINC system:

TL-PINC+Plasmid→TL-PINC::::::Plasmid

Formation of such a targeted complex is also illustrated by the following example of non-covalently attached targeting ligand (TL) to PINC system

TL::::::PINC+Plasmid→TL::::::PINC::::::Plasmid

or alternatively,

PINC+Plasmid→PINC::::::::Plasmid+TL→TL::::::PINC:::::::Plasmid

In these examples :::::::: is non-covalent interaction such as ionic, hydrogen-bonding, Van der Waals interaction, hydrophobic interaction, or combinations of such interactions.

A targeting method for cytotoxic agents is described in Subramanian et al., International Application No. PCT/US96/08852, International Publication No. WO 96/39124, hereby incorporated by reference. This application describes the use of polymer affinity systems for targeting cytotoxic materials using a two-step targeting method involving zip polymers, i.e., pairs of interacting polymers. An antibody attached to one of the interacting polymers binds to a cellular target. That polymer then acts as a target for a second polymer attached to a cytotoxic agent. As referenced in Subramanian et al., other two-step (or multi-step) systems for delivery of toxic agents are also described.

In another aspect, nucleic acid coding sequences can be delivered and expressed using a two-step targeting approach involving a non-natural target for a PINC system or PINC-targeting ligand complex. Thus, for example, a PINC-plasmid complex can target a binding pair member which is itself attached to a ligand which binds to a cellular target (e.g., a MAB). Binding pairs for certain of the compounds identified herein as PINC compounds as identified in Subramanian et al. Alternatively, the PINC can be complexed to a tareting ligand, such as an antibody. That antibody can be targeted to a non-natural target which binds to, for example, a second antibody.

III. Model Systems for Evaluation of Interferon Alpha Constructs and Formulations

In accord with the concept of using interferon alpha expressing plasmid constructs and formulations in anti-cancer treatment, murine model systems were utilized based on murine tumor cell lines. The line primarily used was S.C. VII/SF, which is a cell line derived from murine squamous cell carcinoma (S.C.).

Squamous cell carcinoma of the head and neck begins with the cells lining the oral and pharyngeal cavities. Clinical disease progresses via infiltration and spreads into the underlying tissues and lymphatics. The undifferentiated, in vivo passage tumor line S.C. VII/SF displays this typical growth pattern. In addition, its rapid growth rate provides a relatively short test period for individual experiments. Other murine tumor cell lines include another SCC line KLN-205, a keratinocyte line 1-7, and a colon adenocarcinoma line MC-38.

An optimal model system preferably satisfies the criteria based on having tumor growth rate in vivo (i.e., tumors are ready for treatment in 4-10 days post implant), invasiveness, and local spread similar to those observed in clinical disease, and providing accessibility for experimental treatment. As indicated, the SCC VII/SF cell line was utilized as the primary model system cell line. This cell line typically grows rapidly, resulting in death of untreated syngeneic mice 14-17 days after tumor cell implantation.

This cell line can be utilized in a variety of ways to provide model system suitable for a variety of different tests. Four such possibilities are described below.

First, SCCVII cells can be utilized in cell culture to provide an in vitro evaluation of interferon alpha expression construct and formulation characteristics, such as expression levels and cellular toxicities.

Second, the cells can be implanted subcutaneously in mice. This system can be utilized in tests in which accessibility of the implant site is beneficial. As an example, the method was utilized in evaluations of expression efficiencies based on the expression of chloramphenicol acetyltransferase (CAT).

Third, the cells can be implanted transcutaneously into the fascia of digastric muscle.

Fourth, the cells can be implanted transcutaneously into digrastric/mylohyoid muscles. The important features of models 3 and 4 are shown in the table below.

TABLE I
Comparison of submandibular tumor models

| Feature | Mouse Tumor Model 3 | Mouse Tumor Model 4 |
|---|---|---|
| Tumor implant procedure | 2-4 × 10⁵ cells transcutaneously into | 5 × 10⁵ transcutaneously into |
| | fascia of digastric muscle | digastric/mylohyoid muscles |
| Tumor growth and | Prominent submandibular bulge; | More variable, invasion of |
| invasiveness characteristics | invasion of digastric/mylohyoid | digastric/mylohyoid muscles and |
| | muscles and lymphatics | lymphatics |
| Treatment procedure | Transcutaneous, needle inserted and | Lower jaw skin flap raised to expose |
| (primary treatment) | moved within tumor to produce a 4 | tumor, needle inserted and moved |
| | quadrant distribution of gene | within tumor to produce a 4 quadrant |
| | medicine | distribution of gene medicine |
| Days treated (post-implant) | Day 5, day 10 (both | Day 5 (tumor exposed), day 8 |
| | transcutaneously) | (transcutaneously) |
| Measurement procedure | External calipering 2-3 × per week | First caliper when tumor exposed for |
| | until death | treatment, second caliper at sacrifice |
| Advantages | Non-surgical, closed model allows | Surgical, open model allows direct |
| | larger experiments and more frequent | treatment of exposed tumor; Local |
| | treatments; Sacrifice unnecessary to | inflammation from surgery may |
| | caliper (= more time points) | additionally stimulate immune |
| | | response; More like clinical situation |
| | | for protocol development |
| Disadvantages | Transcutaneous treatment is | Labor intensive; Smaller, fewer |
| | potentially less accurate and | experiments possible; Tumors deeper |
| | intensive; less like expected clinical | and more difficult to treat |
| | treatments than surgical approaches | transcutaneously (for secondary |
| | | treatments); Fewer treatments and |
| | | caliperings possible |

The tumor size treated in the mouse models is generally 20-50 mm³. A 50 mm³ mouse tumor is approximately equivalent to 150 cc³ human tumor having an average diameter of about 6.6 cm. This tumor size is approximately 10-fold larger than the size proposed to be treated in the phase I clinical trials. This indicates that the mouse models are strongly biased towards over estimating the expected tumor burden in human patients.

IV. Formulations for in vivo Delivery

A. General

While expression systems such as those described above provide the potential for expression when delivered to an appropriate location, it is beneficial to provide the expression system construct(s) in a delivery system which can assist both the delivery and the cellular uptake of the construct. Thus, this invention also provides particular formulations which include one or more expression system constructs (e.g., DNA plasmids as described above), and a protective, interactive non-condensing compound.

An additional significant factor relating to the plasmid construct is the percentage of plasmids which are in a supercoiled (SC) form rather than the open circular (OC) form.

B. Delivery and Expression

A variety of delivery methods can be used with the constructs and formulations described above, in particular, delivery by injection to the site of a tumor can be used. The submandibular tumor models utilized injection into four quadrants of the tumor being treated.

C. Anti-Cancer Efficacy of Human Interferon Alpha Formulations

The effects of the administration of the interferon alpha formulations described above were evaluated using the S.C. VII mouse tumor models. Plasmid constructs as described above were incorporated in delivery formulations. The formulations were delivered by injection.

D. Synergistic Effects of Interferon Alpha Plasmid and IL-12 Plasmid and Effect of Human Interferon Alpha Formulation Administration on Production of Secondary Cytokines

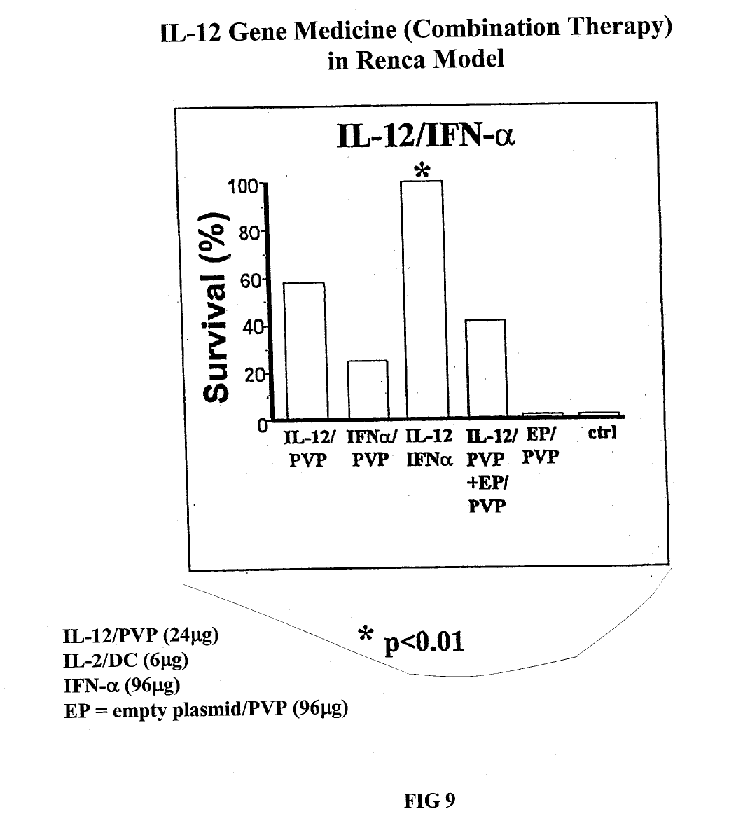

The effects of the expression of the human interferon alpha plasmids in tumor cells on the progress of the mouse tumors demonstrates that such interferon alpha expression is effective against such tumors. However, it was also shown that IL-12 can act synergistically with the interferon alpha expression to exercise the antitumor effect (see FIG. 9).

E. Toxicity Evaluation of Exemplary Formulations

The exemplary formulations do not show high cellular toxicity at the concentrations tested, suggesting that the formulations do not significantly kill cells by direct toxic action in vivo. Moreover, the anti-tumor activity induced by IFNα gene therapy is dependent upon activation of the immune system, which is demonstrated by depletion studies in vivo. Removal of a specific T lymphocyte population (CD8⁺) abrogates the anti-tumor activity elicited by IFNα gene therapy.

V. Administration

Administration as used herein refers to the route of introduction of a plasmid or carrier of DNA into the body. In addition to the methods of delivery described above, the expression systems constructs and the delivery system formulations can be administered by a variety of different methods.

Administration can be directly to a target tissue or by targeted delivery to the target tissue after systemic administration. In particular, the present invention can be used for treating disease by administration of the expression system or formulation to the body in order to establishing controlled expression of any specific nucleic acid sequence within tissues at certain levels that are useful for gene therapy.

The preferred means for administration of vector (plasmid) and use of formulations for delivery are described above. The preferred embodiments are by direct injection using needle injection.

The route of administration of any selected vector construct will depend on the particular use for the expression vectors. In general, a specific formulation for each vector construct used will focus on vector uptake with regard to the particular targeted tissue, followed by demonstration of efficacy. Uptake studies will include uptake assays to evaluate cellular uptake of the vectors and expression of the DNA of choice. Such assays will also determine the localization of the target DNA after uptake, and establishing the requirements for maintenance of steady-state concentrations of expressed protein. Efficacy and cytotoxicity can then be tested.

Toxicity will not only include cell viability but also cell function.

Muscle cells have the unique ability to take up DNA from the extracellular space after simple injection of DNA particles as a solution, suspension, or colloid into the muscle. Expression of DNA by this method can be sustained for several months.

Delivery of formulated DNA vectors involves incorporating DNA into macromolecular complexes that undergo endocytosis by the target cell. Such complexes may include lipids, proteins, carbohydrates, synthetic organic compounds, or inorganic compounds. Preferably, the complex includes DNA, a cationic lipid, and a neutral lipid in particular proportions. The characteristics of the complex formed with the vector (size, charge, surface characteristics, composition) determines the bioavailability of the vector within the body. Other elements of the formulation function as ligand which interact with specific receptors on the surface or interior of the cell. Other elements of the formulation function to enhance entry into the cell, release from the endosome, and entry into the nucleus.

Delivery can also be through use of DNA transporters. DNA transporters refers to molecules which bind to DNA vectors and are capable of being taken up by epidermal cells. DNA transporters contain a molecular complex capable of noncovalently binding to DNA and efficiently transporting the DNA through the cell membrane. It is preferable that the transporter also transport the DNA through the nuclear membrane. See, e.g., the following applications all of which (including drawings) are hereby incorporated by reference herein: (1) Woo et al., U.S. Ser. No. 07/855,389, entitled “A DNA Transporter System and Method of Use, filed Mar. 20, 1992, now abandoned; (2) Woo et al., PCT/US93/02725, International Publ. WO93/18759, entitled “A DNA Transporter System and Method of Use”, (designating the U.S. and other countries) filed Mar. 19, 1993; (3) continuation-in-part application by Woo et al., entitled “Nucleic Acid Transporter Systems and Methods of Use”, filed Dec. 14, 1993, U.S. Ser. No. 08/167,641; (4) Szoka et al. , U.S. Ser. No. 07/913,669, entitled “Self-Assembling Polynucleotide Delivery System”, filed Jul. 14, 1992 and (5) Szoka et al., PCT/US93/03406, International Publ. WO93/19768 entitled “Self-Assembling Polynucleotide Delivery System”, (designating the U.S. and other countries) filed Apr. 5, 1993. A DNA transporter system can consist of particles containing several elements that are independently and non-covalently bound to DNA. Each element consists of a ligand which recognizes specific receptors or other functional groups such as a protein complexed with a cationic group that binds to DNA. Examples of cations which may be used are spermine, spermine derivatives, histone, cationic peptides and/or polylysine. One element is capable of binding both to the DNA vector and to a cell surface receptor on the target cell. Examples of such elements are organic compounds which interact with the asialoglycoprotein receptor, the folate receptor, the mannose-6-phosphate receptor, or the carnitine receptor. A second element is capable of binding both to the DNA vector and to a receptor on the nuclear membrane. The nuclear ligand is capable of recognizing and transporting a transporter system through a nuclear membrane. An example of such ligand is the nuclear targeting sequence from SV40 large T antigen or histone. A third element is capable of binding to both the DNA vector and to elements which induce episomal lysis. Examples include inactivated virus particles such as adenovirus, peptides related to influenza virus hemagglutinin, or the GALA peptide described in the Szoka patent cited above.

Transfer of genes directly into a tumor has been very effective. Experiments show that administration by direct injection of DNA into tumor cells results in expression of the gene in the area of injection. Injection of plasmids containing human interferon alpha results in expression of the gene for 5 days following a single intra-tumoral injection. Human IFNα production was highest in tumors harvested 1 day post-tumor injection and steadily declined thereafter. The injected DNA appears to persist in an unintegrated extrachromosomal state. This means of transfer is a preferred embodiment.

Administration may also involve lipids as described in preferred embodiments above. The lipids may form liposomes which are hollow spherical vesicles composed of lipids arranged in unilamellar, bilamellar, or multilamellar fashion and an internal aqueous space for entrapping water soluble compounds, such as DNA, ranging in size from 0.05 to several microns in diameter. Lipids may be useful without forming liposomes. Specific examples include the use of cationic lipids and complexes containing DOPE which interact with DNA and with the membrane of the target cell to facilitate entry of DNA into the cell.

Gene delivery can also be performed by transplanting genetically engineered cells. For example, immature muscle cells called myoblasts may be used to carry genes into the muscle fibers. Myoblast genetically engineered to express recombinant human growth hormone can secrete the growth hormone into the animal's blood. Secretion of the incorporated gene can be sustained over periods up to 3 months.

Myoblasts eventually differentiate and fuse to existing muscle tissue. Because the cell is incorporated into an existing structure, it is not just tolerated but nurtured. Myoblasts can easily be obtained by taking muscle tissue from an individual who needs gene therapy and the genetically engineered cells can also be easily put back with out causing damage to the patient's muscle. Similarly, keratinocytes may be used to delivery genes to tissues. Large numbers of keratinocytes can be generated by cultivation of a small biopsy. The cultures can be prepared as stratified sheets and when grafted to humans, generate epidermis which continues to improve in histotypic quality over many years. The keratinocytes are genetically engineered while in culture by transfecting the keratinocytes with the appropriate vector. Although keratinocytes are separated from the circulation by the basement membrane dividing the epidermis from the dermis, human keratinocytes secrete into circulation the protein produced.

The chosen method of delivery should result in expression of the gene product encoded within the nucleic acid cassette at levels which exert an appropriate biological effect. The rate of expression will depend upon the disease, the pharmacokinetics of the vector and gene product, and the route of administration, but should be in the range 0.001-100 mg/kg of body weight/day, and preferably 0.01-10 mg/kg of body weight/day. This level is readily determinable by standard methods. It could be more or less depending on the optimal dosing. The duration of treatment will extend through the course of the disease symptoms, possibly continuously. The number of doses will depend upon the disease, delivery vehicle, and efficacy data from clinical trials.

### EXAMPLES

The present invention will be more fully described in conjunction with the following specific examples which are not to be construed in any way as limiting the scope of the invention. As shown below, mIFN-α gene medicine reduces the growth of tumors in syngeneic murine tumor models. Lipid formulations of mIFN-α gene medicine display anti tumor activity in both SCC-VII and MC-38 tumor models. PINC and peptide formulations of mIFN-α gene medicine display anti tumor effects in the MC-38 tumor model. The anti tumor effects of mIFN-α gene medicine are dose dependent. In addition, the examples demonstrate that treatment of tumors with the combination of IFNα and IL-12 gives an unanticipated more than additive (synergystic) anti-tumor activity using either a PINC or a lipid formulation.

### Example 1

A plasmid expression system encoding murine IFNα4 and formulated in a polymeric delivery system was used for in vivo immunotherapeutic activity against an immunogenic murine renal cell carcinoma, Renca, and a non-immunogenic mammary adenocarcinoma, TS/A. Mice bearing established tumors were treated with IFNα/polyvinyl-pyrrolidone (PVP) expression complexes via direct intra-tumoral injection. Up to 100% tumor growth inhibition was observed in the treated mice. By using an optimal dose of 96 and 48 μg of formulated IFN-α plasmid for the treatment of Renca and TS/A respectively, 30% (Renca) and 10% (TS/A) of the treated animals remained tumor-free. Tumor inhibition was dependent upon activation of the immune system. The anti-tumor activity elicited by IFN-α gene therapy was abrogated when mice were selectively depleted of CD8⁺ T cells. By contrast, removal of CD4⁺ resulted in increased tumor rejection following IFN-α/PVP treatments. Finally, mice that remained tumor-free following IFN-α gene therapy displayed immune resistance to a subsequent challenge of tumor. These data provide evidence that non-viral IFNα gene therapy can be used to induce an efficient anti-tumor response.

Local presence of cytokines in tumors can activate an immune response that in some cases leads to induction of specific long-lasting anti-tumor immunity. By direct intra-tumoral injection of plasmid encoding murine IFNα4 and formulated in a polymeric delivery system, tumor-bearing mice develop an immune response, which leads to inhibition and eradication of the tumor. We have shown by depletion studies in vivo that the immune response induced by IFNα is mainly CD8-mediated and that this treatment results in a long-term immunity in mice demonstrating complete tumor regression. Thus, non-viral IFNα gene therapy may be an effective alternative to IFNα protein therapy for human cancers.

Transduction of tumor cells with cytokine genes has proven to be a very efficient technique to induce cytokine-mediated anti-tumor immunity. In experimental models, the local presence of IL-2, IL-1, IL-4, IL-6, IL-7, IL-12, IFNs and CSFs (i.e., GM-CSF) at the site of the tumor can result in significant tumor growth inhibition (Colombo et al., “Local Cytokine Availability Elicits Tumor Rejection and Systemic Immunity Through Granulocyte-T-Lymphocyte Cross-Talk”, Cancer Research, 52, 4853-4857 (1992)). In these systems, cytokines have limited effect on tumor proliferation directly but are capable of activating a rapid and potent anti-tumor immune response, which impedes tumor progression. Established parental tumors, however, are difficult to eradicate with ex vivo cytokine-transduced tumor cells because efficacy of vaccination is highly dependent on the size, growth rate and invasiveness of the tumor.

To overcome these problems, cytokine-based gene therapy approaches, which can deliver transgenic cytokines locally and induce an anti-tumor immune response, have been recently evaluated by a number of investigators (Formi et al., “Cytokine-Induced Immunogenicity: From Exogenous Cytokines to Gene Therapy”, Journal of Immunotherapy, 14, 253-257, (1993); Pericle et al., “An Efficient Th2-type Memory Follows Cd8+ Lymphocyte-driven and Eosinophil-mediated Rejection of a Spontaneous Mouse Mamary Adenocarcicoma Engineered to Release II-4”, The Journal of Immunology, 153, 5660-5673. (1994); Pardoll et al., “Gene Modified Tumor Vaccines, In Cytokine-Induced Tumor Immunogenicity”, eds. Academic Press, London, p. 71-86. (1994); and Musiani et al., “Cytokines, Tumor-cell Death and Immunogenicity: A Question of Choice”, Immunology Today. 1, 32-36 (1997)). Technological breakthroughs in gene therapy using adenoviral, retroviral, and liposomal vectors have provided powerful tools with which to study the biological effects of specific cytokine mediators as well as to develop novel and clinically applicable anti-tumor immunotherapies (Pardoll, “Paracrine Cytokine Adjuvants in Cancer Immunotherapy”, Annu. Rev. Immunol. 13, 399-415 (1995); Bramson et al., “Direct Intratumoral Injection of an Adenovirus Expressing Interleukin-12 Induces Regression and Long-lasting Immunity That Is Associated with Highly Localized Expression of Interleukin-12”, Hum. Gene Ther., 7, 1995-2002 (1996); Rao et al., “II-12 Is an Effective Adjuvant to Recombinant Vaccinia Virus-based Tumor Vaccines”, J. Immunol. 156, 3357-3365. 1996; Rakhmilevich et al., “Gene Gun-mediated Skin Transfection with Interleukin 12 Gene Results in Regression of Established Primary and Metastatic Murine Tumors”, Proc. Natl. Acad. Sci. USA. 93, 6291-6296 (1996); and Rakhmilevich et al, “Cytokine Gene Therapy of Cancer Using Gene Gun Technology: Superior Antitumor Activity of Interleukin-12”, Hum. Gene Ther. 8, 1303-1311, (1997)).

A gene therapy approach utilizing an interactive polymeric gene delivery system that increases protein expression by protecting plasmid DNA (pDNA) from nucleases and controlling the dispersion and retention of pDNA in muscle cells is described in Mumper et al., 1996. These polymeric interactive non-condensing (PINC) systems routinely result in a greater amount of gene expression from tissues as compared to delivery of unformulated plasmid in saline (Mumper et al., 1996). By using a plasmid that encodes human insulin growth factor-i (hIGF-1) and formulated as a PINC complex, production of biologically active h IGF-1 in vivo following intramuscular injection has been shown (Alila et al., “Expression of Biologically Active Human Insulin-Like Growth Factor-i Following Intramuscular Injection of a Formlated Plasmid in Rats”, Human Gene Therapy, 8, 1785-1795 (1997)). The specific objective of this study was to determine whether a plasmid expression system encoding murine IFNα4 and formulated as a complex with PVP could induce an anti-tumor immune response following direct injection into subcutaneous murine tumors.

The IFN family consists of three major glycoproteins, IFNα, IFNβ and IFNγ. Although IFNs were first developed as antiviral agents, it is now clear that they also control cell growth and differentiation, and modulate various aspects of host immunity (Gresser et al., “Antitumor effects of interferon”, Acta Oncol. 28, 347-353 (1989)). Clinical data concluded that systemic chronic administration of IFNα could produce regression of vascular tumors, including Kaposi's sarcoma, pulmonary hemangiomastosis, and hemangiomas (Singh et al., “Interferons A and B Down-regulate the Expression of Basic Fibroblast Growth Factor in Human Carcicomas”, Proc. Natl. Acad. Sci. USA. 92, 4562-4566 (1995)). Although IFNα was the first cytokine to be used in clinical trials that proved to be effective against certain types of human cancer, only recently has this cytokine been considered as a candidate for gene therapy (Ogura et al. 1993, Belldegrun et al., “Human Renal Carcinoma Line Transfected With Interleukin-2 and/or Interferon α Gene(s): Implications for Live Cancer Vaccines, Journal of the National Cancer Institute, 85, 207-216 (1993).

Initial studies have shown that the injection of genetically modified tumor cells producing IFNα into syngeneic mice induces tumor growth inhibition and elicits a tumor-specific immune memory (Ferrantini et al., Interferon Alpha-1-Interferon Gene Transfer into Metastatic Friend Lukemia Cells Abrogated Tumorigenicity in Immunocompetent Mice: Antitumor Therapy by Means of interferon-Producing Cells; Cancer Res. 53, 1107-4615 (1993); Ferrantini et al., “Ifn-α1 Gene Expression into a Metastatic Murine Adenocarcicoma (Ts/a) Results in Cd8+ T Cell-Mediated Tumor Rejection and Development of Antitumor Immunity: Comparative Studies with Ifn-α-producing Ts/a Cells” Journal of Immunology, 153, 4604-4615, (1994); Musiani et al. 1997). However, the real value of this potential form of vaccine in inducing the regression of established tumors remains to be demonstrated.

In this study we present evidence that direct injection of IFNα plasmid formulated in PVP into subcutaneous murine tumors results in a host-dependent tumor rejection, primarily mediated by CD8⁺ T cells, and elicits a protective immunity against subsequent tumor re-challenge.

Materials and Methods

Plasmid Construction and Formulation.

A plasmid expression system containing an expression cassette for mIFN-α4 was constructed as follows. The coding sequence of the murine IFN-α4 gene (Genebank X01973 M15456 M23830 X01967) was amplified by PCR from mouse genomic DNA. The amplified mIFN-α4 sequence was then subcloned into a plasmid backbone, and the sequence fidelity was verified by DNA sequence analysis (data not shown). The coding sequence for mIFN-α4 was then subcloned as an XbaI-BamH1fragment into the expression plasmid pIL0697 to create the mIFN-α4 expression system pIF0836. Plasmid pVC0612 (empty plasmid, EP) contains expression elements including the cytomegalovirus immediate early promoter and the 3′ UTR/poly(A) signal from the bovine growth gene in the pVC0289 backbone described by Alila et al. (1997). Plasmid pVC0612 was used as a control plasmid in all in vivo experiments. Plasmids for intra-tumoral injection were grown under kanamycin selection in E. coli host strains DH5α and purified using conventional alkaline lysis and chromatographic methods. Purified plasmid utilized for intra-tumoral injections had the following specifications: endotoxin (<500 Eu/mg plasmid); protein (<1%); and chromosomal DNA (<20%). Purified pIF0836 and control plasmids were formulated at a concentration of 3 mg DNA/ml in a solution of 5% w/v polyvinyl-pyrrolidone (Plasdone C-30, ISP Technologies, Wayne, N.J.), 150 mM NaCl on the day of injection, as described previously (Mumper et al., 1996).

Western Blot Analysis and Bioassay for mIFNα.

HeLa cells were plated in 6 well plates at 3×10⁵ cells per well, and transfected using 1 μg of mouse IFNα4 plasmid pIF0836C and 3 μg of Lipofectamine (Life Technologies, Inc., Gaithersburg, Md.) in serum-free DMEM. Same supernatants were harvested 24 hours later and immunoprecipitated using anti-mouse interferon α/β polyclonal antibody (BioSource International, Camarillo, Calif.) and protein A and G agarose (Boehringer Mannheim, Indianapolis, Ind.). Samples were run on a 12% Tris-glycine gel and electroblotted to Millipore PVDF membrane. Anti-mouse interferon α/β polyclonal antibody was used at 1:1000, followed by anti-sheep Ig HRP (Boehringer Mannheim) at 1:1000. Biotinylated molecular weight markers were detected using Streptavidin-HRP (Amersham, Arlington Heights, Ill.). Detection was performed using the Amersham ECL kit. Supernatants were also tested for IFNα biological activity using L929 cells treated with encephalomyocarditis virus, in parallel with a NIH mouse IFNα reference reagent (Access Biomedical, San Diego, Calif.).

Animals

Normal 8-week-old female BALB/c mice were purchased from Harlan Laboratories, Houston, Tex. Mice were maintained on ad libitum rodent feed and water at 23° C., 40% humidity, and a 12-h/12-h light-dark cycle. Animals were acclimated for at least 4 days before the start of the study.

Tumors

Three established mouse tumor models were used in this study. TS/A is a tumor cell line established by Dr. P. Nanni, University of Bologna, Italy, from the first in vivo transplant of a moderately differentiated mammary adenocarcinoma that spontaneously arose in a BALB/c mouse (Nanni et al., 1983). A number of pre-immunization-challenge experiments suggested that TS/A does not elicit a long-lasting anti-tumor immunity (Fomi et al., 1987). TS/A was generously provided by Dr. Guido Fomi, University of Turin, Italy. Renca, a spontaneously arising murine renal cell carcinoma, and CT-26, a colon adenocarcinoma, were generously provided by Dr. Drew M. Pardoll, John Hopkins Hospital, Baltimore, Md. Tumor cell cultures were maintained in sterile disposable flasks from Coming (Corning, N.Y.) at 37° C. in a humidified 5% CO₂ atmosphere, using RPMI 1640 supplemented with 10% FBS, 100 U/ml penicillin, 100 U/ml streptomycin and 50 μg/ml gentamycin; all from Life Technologies.

In vivo Evaluation of Tumor Growth and Treatments

BALB/c mice were challenged s.c. in the middle of the left flank with 30 μl of a single-cell suspension contained the specified number of cells. Seven days later when the tumor size reached approximately 10 mm³, treatments with IFNα/PVP or EP/PVP started and were repeated at 1-2 day intervals for 2 weeks (total of 8 treatments: 4/week). Tumor volume was measured with electronic caliper in the two perpendicular diameters and in the depth. Measurements of the tumor masses (mm³) were performed twice a week for 40-50 days. All mice bearing tumor masses exceeding 1 cm³ volume were sacrificed for humane reasons. When depletion of immunocompetent cells in vivo was required, a group of mice received i.v 0.5 ml of α-CD4 (GK1.5 hybridoma, 207-TIB, ATCC, Rockville, Md.) ascite (1:10), or α-CD8 (2.43 hybridoma, 210-TIB, ATCC) ascite (1:100) or i.p. 100 μg α-GR1 (RB6-8C5 hybridoma, Pharmingen, San Diego, Calif.). Control mice received i.v. 0.5 ml isotype control IgG (Pharmingen). Antibody treatments were performed twice: first injection 1 day before starting the gene therapy treatment and the second injection (i.p at the same dosage) 7 days later.

CTL Assay.

A standard 6-hour 51-chromium (⁵¹Cr)-release assay was performed following 5 days of in vitro effector cell stimulation. Single cell suspensions of splenocytes were prepared 3 weeks following tumor challenge by mashing the spleens in RPMI 1640 medium (Life Technologies) and passing the cells through 70 μm nylon mesh cell strainers (Falcon, Becton Dickinson, Lincoln Park, N.J.) into 50 ml centrifuge tubes (Falcon). After centrifugation, red blood cells were lysed with ACK Lysing Buffer (Biofluids, Inc., Rockville, Md.) and the splenocytes washed twice with RPMI. In vitro stimulation cultures contained 3×10⁶ splenocytes/effectors per ml with 6×10⁵ mitomycin-C-treated Renca/stimulator cells per ml and 10 Units per ml recombinant murine IL-2 (Genzyme, Cambridge, Mass.) in RPMI containing 10% FBS, 22 mM HEPES buffer (Research Organics Inc., Cleveland Ohio), Penn-Streptomycin, 5×10⁻⁵ M 2-β-mercapto-ethanol (Life Technologies), OPI media supplement (Sigma, St. Louis, Mo.), and essential and non-essential amino acids (Life Technologies) (for a 5:1 responder: stimulator ratio). Stimulators were prepared by incubating Renca cells at 3×10⁷ per ml in RPMI with 30 μg per ml mitomycin-C (Sigma) at 37° C. for 60 minutes, followed by four washes in HBSS with 2.5% FBS. After 5 days at 37° C., effector cells were pelleted, resuspended in complete RPMI, counted, and mixed with ⁵¹Cr-labeled targets in a 96 well V-bottomed plate (Costar/Corning, Cambridge, Mass.). Renca and CT26 targets were labeled by incubating them at 2×10⁶ cells per ml in complete RPMI with 150 uCi ⁵¹Cr (Amersham) for 2.5 hours. Targets were washed 3 times in HBSS with 2.5% FBS and resuspended in complete RPMI before addition to the assay. After mixing effectors and targets (in triplicate wells) and a brief pelleting, plates were placed at 37° C. for 6 hours. Approximately 90% of the supernatants were then collected from each well with the Skatron Harvesting Press and Supernatant Collection System (Skatron Instruments, Norway). ⁵¹Cr release was detected using a WALLAC 1470 Wizard automatic gamma counter (WALLAC Inc., Gaithersburg Md.). Specific release was determined with the following equation: (experimental cpm−spontaneous cpm)/(total cpm−spontaneous cpm)×100. Spontaneous release from the targets was less than 18%, and the standard error of the triplicate experimental counts was less than 14%.

Statistical Analysis.

Data for the effects of mIFN-α. gene therapy on tumor growth were analyzed by repeated measures analysis. Individual treatment means were compared using Duncan's multiple range test when the main effect was significant. Data for the effect of mIFN-α gene therapy on tumor rejection were analyzed by ANOVA. In all cases a p value of less than 0.05 was considered to be statistically significant.

Results

Expression of mIFN-α

Murine IFN-α expression plasmid (pIF0836) was transfected into Cos-1 cells, and the resulting conditioned media was assayed for mIFN-α by Western blot and by bioassay. Western blot analysis of conditioned media indicated that the recombinant mIFN-α expressed from pIF0836 template was present as a single band with an approximate molecular weight of 23 kDa. This band was not observed in conditioned media from mock-transfected cells and likely represents a glycosylated form of mIFN-α. Recombinant mIFN-α ran with an approximate molecular weight of 18 kDa, which corresponds to the predicted molecular weight of non-glycosylated mIFN-α. Assay of conditioned media using an anti-viral bioassay for mIFN-α indicated that approximately 175×10³ IU/ml mIFN-α were present.

Anti-tumor activity of IFN-α gene therapy. The anti-tumor effect of murine IFNα4 plasmid formulated as a complex with PVP (IFNα/PVP) was tested in a syngeneic murine renal cell carcinoma (Renca) and a mammary adenocarcinoma (TS/A) tumor model. BALB/c mice were challenged subcutaneously with 7×10⁵ Renca or 1×10⁵ CT26 cells, and IFNα/PVP injections were initiated 7 days later when tumors reached approximately 10 mm³ size. Each group of mice received at interval of 1-2 days 8 treatments (4 injections/week) of IFNα/PVP at scalar doses (from 12 to 96 μg/mouse), EP/PVP (96 μg/mouse) or no treatments for control (ctrl). Tumor size increased progressively in mice injected with EP/PVP (Renca, TS/A) or low doses of IFNα/PVP (TS/A), while tumors in mice injected with each dose of IFNα/PVP (Renca) or high dose of IFNα/PVP (TS/A) showed marked growth inhibition.

Tumor Growth Inhibition is Associated to Systemic Immune Response.

Treatments of Renca and TS/A tumors with IFNα/PVP at 96 μg/mouse and 48 μg/mouse respectively, induced complete regression in 6 out of 20 (Renca) and 2 out of 20 (TS/A) of challenged mice. To test whether the rejection of these tumors leads to specific immune memory, mice with no tumors for 40-50 days following IFNα treatments were re-challenged with a greater number of fresh tumors in the right flank. All mice that rejected primary tumors displayed protection against the second tumor challenge whereas mice used as the control group and injected for the first time with same number of tumor cells (1×10⁶ Renca or 2×10⁵ TS/A) developed tumors.

To evaluate the requirements for the induction of anti-tumor immune memory, Renca and TS/A were injected into BALB/c rendered immunosuppressed by treatment with anti-CD4, anti-CD8 or anti-polymorphonuclear cells (PMN). Depletion of CD8⁺ T cells allowed both Renca and TS/A to grow in all animals following IFNα/PVP treatments, showing that this population is crucial for the immune response induced by IFNα gene therapy. No increase in tumor growth was found in mice treated with anti-PMN (α-GR1) monoclonal Ab (mAb). Increase in tumor rejection was observed in mice depleted of CD4⁺ T and treated with IFNα/PVP suggesting that depletion of CD4⁺ T cells can enhance the anti-tumor effect of IFNα gene therapy.

Expression of IFN-α within the tumor induces a CTL response. To assess whether CD8⁺ tumor specific CTL were induced in vivo by IFNα/PVP treatments, splenocytes from Renca tumor-challenged mice were tested for their cytolytic activity following IFNα gene therapy. Cytotoxic activity against Renca, and not against CT26 cells used as control for tumor specificity, was found in 2 of 4 mice that had received IFNα gene therapy. Moreover, splenocytes from mice depleted of CD4⁺ T cells and treated with IFNα/PVP demonstrated potent CTL activity against Renca cells. By contrast, little CTL activity against Renca cells was evident from splenocytes isolated from mice treated with EP/PVP.

Discussion

The data reported herein demonstrate that direct administration of IFNα gene formulated in a polymeric delivery system into subcutaneous renal cell carcinoma and mammary adenocarcinoma inhibits tumor growth and induces a long-lasting immunity to secondary tumor challenges. Of considerable significance is the fact that the anti-tumor response was observed against both an immunogenic carcinoma as well a more aggressive and poorly immunogenic adenocarcinoma, a phenotype which is similar to many spontaneously arising tumors in man.

A variety of genetic abnormalities arise in human cancers that contribute to neoplastic transformation and malignancy. Despite increasing understanding of the molecular basis of cancer, many malignancies remain resistant to established forms of treatment. More recently, molecular genetic interventions have been designed in an attempt to improve the efficacy of immunotherapy. While numerous experimental studies have been performed in murine models with tumor cells transduced with cytokine-gene ex vivo, a major limitation in the translation of this strategy to large-scale human tumor vaccine therapy is the labor intensity and variability of establishing each individual tumor in culture and transducing it with an appropriate vector (i.e., retrovirus). Our approach to this problem is to use a non-viral delivery system to modify tumor cells in vivo with cytokine cDNAs so that the tumor cells can supply the cytokine of interest in a paracrine fashion to the anti-tumor responder cells present within the tumor.

Using a plasmid containing IFNα4 gene and formulated in PVP, we have shown that intra-tumoral injections of this DNA-PINC complex can lead to complete tumor regression in 30% of the cases (Renca model) with an overall response rate of 100% tumor growth inhibition. These results are in agreement with a recent study that described an anti-tumor activity elicited by genetically modified TS/A cells producing murine IFN-α1 (Ferrantini et al., 1994). Although the anti-tumor effect of IFNα using cytokine-gene transduced tumor cells has been described (Scarpa et al., “Extracellular Matrix Remodelling in a Murine Mamary Adenocarcicoma Transfected with the Interferon-alpha1 Gene”, Journal of Pathology. 181, 116-123 1997), the real value of IFNα gene therapy in blocking or inhibiting advanced tumors remains to be explored. The advantage of using a non-viral IFNα gene delivery system over retrovirus is that tumor cells could be transduced directly in vivo without the need of first establishing tumor cells in vitro. Moreover, IFNα has a potent anti-viral activity limiting the use of this gene in combination with viral vectors.

Therapeutic levels of gene expression for IGF-I using a similar interactive PVP-based delivery system have previusly been described (Alila et al., 1997). Direct intra-tumor injection of the same PINC delivery system as a complex with IFNα gene, resulted in dispersion in vivo of plasmid into target cells inducing an IFNα-specific anti-tumor activity. Tumors treated with the same plasmid but in the absence of IFNα coding region and formulated as a complex with PVP, did not respond to this treatment and grew in a similar rate to untreated tumors. By using an optimal dose of IFNα/PVP, tumor-bearing mice were able to reject the tumors mounting a specific long-lasting tumor immunity. Although, the numbers of mice rejecting a second tumor challenge was low, this observation indicates that a considerable portion of the activity of IFNα in inducing the rejection of established tumors is not anti-angiogenic or anti-proliferative but immunostimulatory. Our result demonstrating that IFNα-induced regression of advanced tumors was prevented by in vivo administration of anti-CD8 mAb provides direct evidence for a key role of CD8⁺ T cells in the anti-tumor effect of IFNα.

Depletion of CD4⁺ T cells in tumor-bearing mice treated with IFNα gene therapy significantly enhanced the therapeutic effect of IFNα, resulting in tumor regression and prolonged survival of up to 80% of treated mice. A CD4-mediated suppression during tumor progression has been previously reported and it has also been shown that depletion of CD4⁺ T cells in tumor-bearing mice results in augmentation of anti-tumor therapy with either IL-2 or IL-12 (Rackmilevich et al., 1994 and Martinotti et al., “Cd4 T Cells Inhibit in Vivo the Cd8-Mediated Immune Response Against Murine Colon Carcinoma Cells Transducted with Interleukin-12 Genes”, Eur. J. Immunol, 25, 137-146. (1995)). They have shown that depletion of CD4⁺ T cells in tumor-bearing mice in the absence of treatment did not affect the growth of tumor itself suggesting that removal of CD4⁺ T cells does not deprive the tumor of growth factors (Rackmilevich et al., 1994). A possible explanation for this phenomenon is that depletion of CD4⁺ T cells from tumor-bearing mice augments the anti-tumor efficacy of IFNα-activated CD8⁺ T cells by releasing them from immunosuppression. The mechanism driving CD4 suppression is poorly understood, although Th2 type cytokines, directly or through B cell activation, may inhibit cell-mediated immunity (Mossmani et al., 1989; Powrie et al., Eur-J-Immunol, 23(11):3043-9 (1993)). CTL can be generated in both CD4-depleted and non-depleted mice from lymphocytes obtained from spleens by in vitro re-stimulation with mitomycin-treated Renca cells and rIL-2. Thus, CD4-mediated suppression appears to be exerted on CD8 expansion and not priming. In accord with the in vivo results, stronger CTL activity was observed from mice depleted of CD4 and treated with IFNα/PVP suggesting CD4⁺ T cells inhibit an IFNα-mediated CD8⁺ T cell response in vivo. This study suggests that direct administration of cytokine genes, like IFNα, into tumors, which have been found to suppress malignancy growth, provide a new therapeutic option for the treatment of human cancers.

### Example 2

### Pharmacology of mIFN-α Gene Medicine in Syngeneic Tumor Models

Gene expression systems encoding either mIFN-2 or mIFN-4 were tested for anti tumor activity when formulated in either cationic lipid, peptide, or PINC delivery systems and injected intratumorally into subcutaneous squamous cell carcinoma (SCC-VII) or adenocarcinoma (MC-38) tumors.

Experimental Design and Treatment Regimen

Experiments to test the anti tumor activity of mIFN-α gene medicine were conducted in either SCC-VII or MC-38 tumor models. Tumor cells (4×10⁵) were injected subcutaneously into the flank region of mice, and treatment was initiated when tumor volume reached approximately 50 mm³. Treatment was begun approximately 6 days (SCC-VII tumors) and 10 days (MC-38 tumors) after tumor initiation and repeated at 3 to 5 day intervals.

All formulations of mIFN-α gene medicine were administered in a dose volume of 50 μl. The faster growing SCC-VII tumors typically received 3 treatments, whereas the relatively slower growing MC-38 tumors typically received 4 treatments. Experiments were terminated when lactose vehicle control tumors reached approximately 1000 mm³.

The anti-tumor effects of murine IFN gene medicine (IFNα/PVP) was tested in syngeneic murine renal cell carcinoma (Renca) and mammary adenocarcinoma (TS/A) tumor model. BALB/c mice were challenged subcutaneously with 7×10⁵ or 1×10⁵ CT26, and IFNα/PVP injections were initiated 7 days later when tumors reached approximately 10 mm³ size. Each group of mice received 8 treatments (4 injections for 2 weeks) of IFNα/PVP at scalar doses (from 12 to 96 μg/mouse), empty plasmid/PVP (EP/PVP, 96 μg/mouse) or no treatments for control (ctrl). Tumor size increased progressively in mice injected with EP/PVP (Renca, TS/A) or low doses of IFNα/PVP (TS/A), while tumors in mice injected with each dose of IFNα/PVP (Renca) or high dose of IFNα/PVP (TS/A) showed marked growth inhibition.

### Example 3

### mIFN-α Gene Medicine Formulated in Cationic Lipid Reduces the Growth of SCC-VII Tumors

Experiments were conducted in the SCC-VII tumor model as described in the preceding example. mIFN-α gene medicine formulated in cationic lipid, peptide, and PINC delivery systems was tested. Results show that cationic lipid formulations significantly reduce the growth of SCC-VII tumors relative both to lactose vehicle injected tumors and to tumors injected with control (non coding) plasmid formulated in cationic lipid. The effect of mIFN-α gene medicine formulated in cationic lipid is dose dependent and there is no effect of mIFN-α gene medicine when formulated in PVA. In addition, analysis of tumors from this experiment using immunohistochemical methods revealed infiltration of CD8+ lymphocytes in tumors injected with cationic lipid formulations, but not in tumors injected with PVA formulations.

mIFN-α gene medicine significantly reduces the growth of SCC-VII tumors as compared to control plasmid or lactose injected tumors. Differences between control plasmid and mIFN-α plasmid were consistent across formulation. Plasmid dose was 46 μg/treatment. Growth of tumors injected with control plasmid was compared to that of tumors injected with mIFN-α gene medicine using repeated measures analysis. mIFN-α reduced SCC-VII tumor growth relative to control plasmid (p=0.035).

### Example 4

### mIFN-α Gene Medicine Reduces the Growth of MC-38 Tumors

Experiments were carried out as described in the preceding examples. The effects of various prototype formulations of mIFN-α gene medicine on the growth of subcutaneous MC-38 tumors were compared. mIFN-α gene medicine elicited reduced tumor growth in all formulations tested (cationic lipid, peptide, and PINC). Subsequent experiments in the MC-38 tumor model have shown similar results.

### Example 5

### Dose Responses

After demonstrating anti tumor effects of mIFN-α gene medicine, the dose response for these effects was investigated in the MC-38 tumor model. Both cationic lipid (DOTMA:Chol) and PINC (PVA) delivery systems were evaluated. Results clearly show that mIFN-α gene medicine elicited a dose dependent reduction in tumor growth. Tumor volume in this experiment was maximally reduced by approximately 50% with mIFN-α/DOTMA:Chol and 60% with mIFN-α/PVA after 4 treatments. Maximal reduction in tumor volume was observed at a plasmid dose of approximately 50 μg/treatment (cumulative dose of approximately 200 μg). These experiments will be conducted primarily in the MC-38 tumor model because it provides a broader treatment window than does the SCC-VII model.

### Example 6

### IFN-Alpha Formulations

The formulations for the IFN-α are: (1) PVP 4 vial, (2) PVP three vial, (3) PVP two vial. The details are listed below:

PVP 4 Vial

Materials: 25% PVP (50 kDa) stock solution, plasmid stock solution, 5 M NaCl stock solution, and water.

Method: Add in order of water, plasmid, 25% PVP and 5 M NaCl into a vial to make a plasmid in 5% PVP in saline formulation. The final concentration of PVP and NaCl are fixed (5% and 150 mM) and plasmid concentration could be varied (but based on the IGF-1 data, 3 mg DNA/ml in 5% PVP in saline should be the best formulation). The quality of the formulation is characterized by pH, DNA concentration, osmolality, and gel electrophoresis. The DNA concentration could be varied from 0.1-5 mg/ml. The pH may be varied from 3-5, osmolality may be 250-400 mOsm.

Three Vial

Material: lyophilized PVP, plasmid stock solution (4 mg/ml), 115 mM Na-Citrate/5% NaCl stock buffer (pH=4).

Method: Add in order of plasmid and buffer into the PVP to make final 3 mg DNA/ml in 5% PVP in 25 mM citrate/saline buffer (pH=4). DNA expression is higher in saline than in the citrate buffer.

Two Vial

Materials: Co-lyophilized plasmid and PVP, saline. Add saline into the co-lyophilized DNA and PVP to make final 3 mg/ml DNA in 5% PVP in saline.

The final formulation is 3 mg/mL DNA, 5% PVP as a single vial. The formulation is prepared by adding (5%) PVP to (4 mg/mL) DNA and recirculating the two components for a finite period of time (using static mixer). Then the formulation is lyophilized and rehydrated with 0.9% sodium chloride, to obtain a final composition of 3 mg/mL, 5%PVP in saline.

### Example 7

### Treatment of Human Tumors

The murine studies are predictive of the response of Human tumors to therapy using a plasmid construct encoding the human IFN alpha gene sequence such as that depicted in SEQ ID NO: 10, 11 or 12. A patient in need of anti-cancer therapy is injected with up to 3 mg of plasmid formulation at daily intervals. The plasmid formulation may contain INF alpha plasmid alone or optionally a mixture of IFN-alpha encoding plasmid and an additional plasmid coding for a cytokine. The preffered cytokine is IL-12. The treatments are continued and the patient monitored as is the usual practice for anti-cancer chemotherapeutic regimes.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention. All patents and publications mentioned in the specification are indicative of the levels of those skilled in the art to which the invention pertains. All patents and publications are herein incorporated by reference to the same extent as if each individual publication was specifically and individually indicated to be incorporated by reference.

1. A mammalian interferon alpha expression plasmid comprising a promoter and a synthetic 5′ intron transcriptionally linked with an interferon alpha coding sequence and a 3′-untranslated region.

2. The plasmid of claim 1, wherein said synthetic intron comprises: a 5′ splice site having the sequence CAGGTAAGT, a branch point having the sequence TACTAAC, and a 3′ splice site having the sequence Y₁₁NYAGG, where Y=C or T, and N=any base, and wherein the synthetic intron has a total length between 80 and 200 nucleotides.

3. The plasmid of claim 1, wherein said human interferon alpha coding sequence is a synthetic sequence wherein the codons are substantially optimized for a preferred codon usage for highly expressed human genes in accordance with FIG. 3.

4. The plasmid of claim 1, wherein said interferon alpha coding sequence has the nucleotide sequence of SEQ ID NO: 10, 11, or 12.

5. The plasmid of claim 1, wherein said 3′ untranslated region is from a growth hormone gene.

6. The plasmid of claim 5, wherein an ALU repeat or ALU repeat-like sequence is deleted from said 3′ untranslated region.

7. The plasmid of claim 5, wherein said growth hormone is a human growth hormone.

8. The plasmid of claim 2, wherein said plasmid further comprises a 5′ UTR having the sequence of SEQ ID NO. 14, wherein the synthetic intron is inserted between residues 48 and 49.

9. The plasmid of claim 1, wherein said promoter is derived from a CMV promoter.

10. The plasmid of claim 1, wherein said plasmid comprises a nucleotide sequence which is the same as the nucleotide sequence of plasmid pIF0921.

11. The composition of claim 10, wherein an ALU repeat or ALU repeat-like sequence is deleted from said 3′ untranslated region.

12. A composition comprising the plasmid of claim 1 and a protective, interactive non-condensing compound.

13. The composition of claim 12, wherein said protective, interactive non-condensing compound is polyvinyl pyrrolidone.

14. The composition of claim 12, wherein said protective, interactive non-condensing compound is polyvinyl alcohol.

15. The composition of claim 12, wherein said plasmid is in a solution having 10 between 0.5% and 50% PVP.

16. The composition of claim 15, wherein said composition includes about 30% or less PVP.

17. The composition of claim 15, wherein said composition includes about 20% or less PVP.

18. The composition of claim 15, wherein said composition includes about 10% or less PVP.

19. The composition of claim 15, wherein said composition includes about 5% or less PVP.

20. The composition of claim 15, wherein said composition includes about 1% or less PVP.

21. The composition of claim 12, wherein said plasmid is at least about 80% supercoiled.

22. The composition of claim 21, wherein said plasmid is at least about 90% supercoiled.

23. The composition of claim 22, wherein said plasmid is at least about 95% supercoiled.

24. A composition comprising a plasmid encoding an interferon alpha coding sequence formulated together with a non-condensing compound selected from the group consisting of: polyvinyl pyrrolidone, polyvinyl alcohol, a polyvinyl pyrrolidone-polyvinyl alcohol co-polymer, N-methyl-2-pyrrolidone, ethylene glycol, propylene glycol and poloxamer.

25. The composition of claim 24, further comprising a targeting ligand.

26. The composition of claim 25, wherein said targeting ligand is selected from the group consisting of galactosyl residues, fucosal residues, mannosyl residues, carnititine derivatives, monoclonal antibodies polyclonal antibodies, peptide ligands and DNA binding proteins.

27. The composition of claim 25, wherein said targeting ligand binds to receptors on cells.

28. The composition of claim 25, wherein said cells are selected from the group consisting of antigen presenting cells, hepatocytes, monocytes, epithelial cells, endothelial cells and cancer cells.

29. The composition of claim 24, farther comprising a targeting ligand.

30. The composition of claim 29, wherein said targeting ligand is selected from the group consisting of galactosyl residues, fucosal residues, mannosyl residues, carnititine derivatives, monoclonal antibodies polyclonal antibodies, peptide ligands and DNA binding proteins.

31. The composition of claim 29, wherein said targeting ligand binds to receptors on cells.

32. The composition of claim 31, wherein said cells are selected from the group consisting of antigen presenting cells, hepatocytes, monocytes, epithelial cells, endothelial cells and cancer cells.

33. The composition of claim 29, wherein said plasmid comprises the plasmids of claim 1.

34. The composition of claim 29, wherein said human interferon alpha coding sequence is a synthetic sequence having optimal codon usage.

35. The composition of claim 29, wherein said interferon alpha coding sequence has the nucleotide sequence of SEQ ID NO:10, 11 or 12.

36. A method for making a composition of claim 24, comprising the steps of:
a. preparing a DNA molecule comprising a transcriptional unit, wherein said transcriptional unit comprises the interferon alpha coding sequence;
b. preparing the non-condensing compound, and
c. combining said non-condensing compound with said DNA molecule in conditions such that a composition capable of delivering a therapeutically effective amount of the interferon alpha coding sequence to a mammal is formed.

37. The method of claim 36 wherein said DNA molecule is a plasmid, wherein said plasmid comprises a CMV promoter transcriptionally linked with an interferon alpha coding sequence, and a human growth hormone 3′- untranslated region/poly (A) signal.
